(19) United States
(12) Patent Application Publication
(10) Pub. No.: US 2012/0101136 A1
(43) Pub. Date: Apr. 26, 2012

(54) 5-MEMBERED HETEROARYL DERIVATIVES USED AS SPHINGOSINE 1- PHOSPHATE RECEPTOR AGONISTS

(75) Inventors: Guanghui Deng, Shanghai (CN); Xichen Lin, Shanghai (CN); Feng Ren, Shanghai (CN); Jia-Ning Xiang, Shanghai (CN)

(73) Assignee: GLAXO GROUP LIMITED

(21) Appl. No.: 13/379,498

(22) PCT Filed: Jun. 24, 2010
(86) PCT No.: PCT/CN10/00940

§ 371 Date: Dec. 20, 2011

(30) Foreign Application Priority Data
Jun. 26, 2009 (GB) 0911130.3

(51) Int. Cl. A61K 31/433; A61K 31/425; C07D 285/08; C07D 271/06; C07D 233/61; A61P 9/00; A61P 25/00; A61P 37/00; A61P 29/00; A61P 41/00; A61P 19/02; A61P 1/00; A61P 17/06; A61P 35/00; A61P 35/04; A61P 3/10; A61K 31/4245

(52) U.S. Cl. 514/361; 514/364; 514/365; 548/128; 548/131; 548/343.5

(57) ABSTRACT

5-membered heteroaryl derivatives of formula (I) or salts thereof, processes for their preparation, pharmaceutical compositions containing them and their uses in the treatment of various disorders mediated by S1P1 receptors are disclosed.

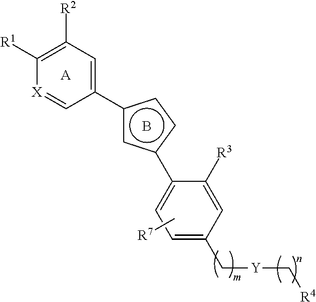

The present invention relates to novel compounds having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandela et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 EurJ Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna at al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024,922 and WO07/116,866.

The following patent application describes indole-oxadiazole derivatives as antipicornaviral agents: WO96/009822. The following patent applications describe indole-carboxylic acid derivatives as leukotriene receptor antagonists, pesticides and agrochemical fungicides respectively: WO06/090817, EP 0 439 785 and DE 39 39 238.

International patent applications WO08/074,821 and WO08/76356 describe oxadiazole-indole derivatives as S1P1 agonists.

A structurally novel class of compounds has now been found which provides agonists of the S1P1 receptor.

The present invention therefore provides compounds of formula (I) or a salt thereof:

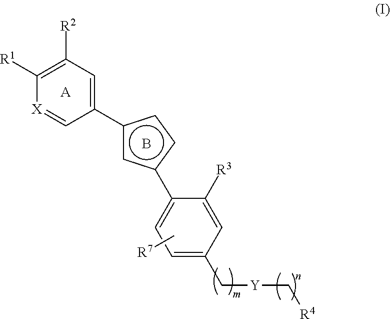

wherein

### X is CH or N;

B is a 5-membered heteroaryl ring selected from:

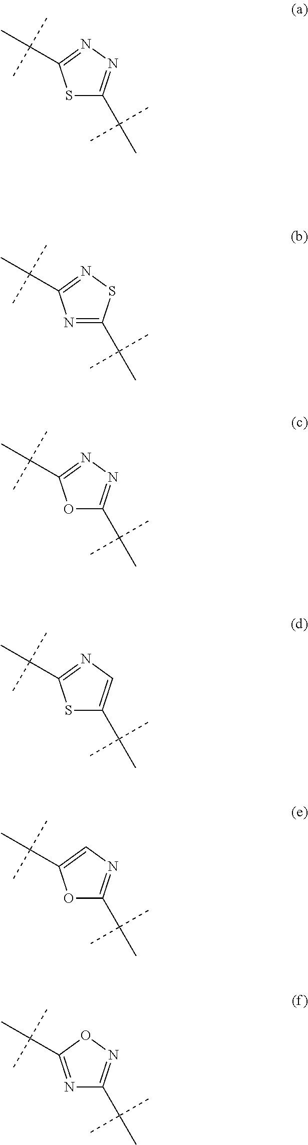

### Y is O or S;

m is 0 to 4;
n is 1 to 4;
R¹ is C₍₁₋₄₎alkoxy;
R² is cyano or chloro;
R³ is C₍₁₋₅₎alkyl, C₍₁₋₅₎alkoxy, halogen, hydrogen, trifluoromethyl or CN;

### R⁴ is COOH, NR⁵R⁶ or OR⁸;

one of R⁵ and R⁶ is C₍₁₋₃₎alkyl and the other is hydrogen or C₍₁₋₃₎alkyl;
R⁸ is C₍₁₋₃₎alkyl;
R⁷ is C₍₁₋₃₎alkyl, C₍₁₋₃₎alkoxy, halogen or hydrogen; and
when m is 1 to 4 and n is 1 to 4 the alkyl groups which they represent may be optionally substituted by C₍₁₋₃₎alkyl or OH.

In one embodiment X is CH. In another embodiment X is N.

In one embodiment B is (a), (b), (c), (d) or (f).

In one embodiment Y is O.

In one embodiment m is 0.

In one embodiment n is 1 to 4.

In one embodiment R¹ is C₍₁₋₄₎alkoxy. In another embodiment R¹ is isopropoxy.

In one embodiment R² is cyano. In another embodiment R² is chloro.

In one embodiment R³ is C₍₁₋₃₎alkyl, C₍₁₋₃₎alkoxy, halogen or hydrogen. In another embodiment R³ is hydrogen, methyl, ethyl, chloro, fluoro or methoxy.

In one embodiment R⁴ is COOH, NR⁵R⁶ or OR⁸. In another embodiment R⁴ is COOH.

In a further embodiment R⁴ is NR⁵R⁶.

In one embodiment R⁵ is hydrogen, methyl, ethyl, propyl or isopropyl and R⁶ is hydrogen or methyl. In another embodiment both R⁵ and R⁶ are methyl.

In one embodiment R⁷ is hydrogen.

In one embodiment R⁸ is methyl.

In one embodiment

### X is CH or N;

### B is (a), (b), (c), (d) or (f);

### Y is O;

m is 0;
n is 1 to 4;
R¹ is isopropoxy;
R² is cyano or chloro;
R³ is hydrogen, methyl, ethyl, chloro, fluoro or methoxy;

### R⁴ is COOH, NR⁵R⁶ or OR⁸;

R⁵ is hydrogen, methyl, ethyl, propyl or isopropyl;
R⁶ is hydrogen or methyl;
R⁷ is hydrogen; and
R⁸ is methyl.

In one embodiment

### X is CH;

### B is (f);

### Y is O;

m is 0;
n is 1-4;
R¹ is C₍₁₋₃₎alkoxy;
R² is cyano or chloro;
R³ is C₍₁₋₃₎alkyl, C₍₁₋₃₎alkoxy, halogen or hydrogen;

### R⁴ is COOH, NR⁵R⁶ or OR⁸;

R⁵ is hydrogen or methyl;
R⁶ is methyl;
R⁷ is hydrogen; and
R⁸ is methyl.

In one embodiment

### X is CH;

### B is (f);

### Y is O;

m is 0;
n is 1-4;
R¹ is C₍₁₋₃₎alkoxy,
R² is cyano or chloro;
R³ is C₍₁₋₃₎alkyl, C₍₁₋₃₎alkoxy, halogen or hydrogen;

### R⁴ is COOH and

R⁷ is hydrogen

In another embodiment

### X is CH;

### B is (f);

### Y is O;

m is 0;
n is 1-4;
R¹ is isopropoxy;
R² is cyano or chloro;
R³ is hydrogen, methyl, chloro or methoxy; and

### R⁴ is COOH; and

R⁷ is hydrogen

In another embodiment

### X is CH;

### B is (f);

### Y is O;

m is 0;
n is 2 to 3;
R¹ is isopropoxy;
R² is cyano or chloro;
R³ is methyl;

### R⁴ is NR⁵R⁶;

R⁵ is hydrogen or methyl;
R⁶ is methyl; and
R⁷ is hydrogen

The term “alkyl” as a group or part of a group e.g. alkoxy or hydroxyalkyl refers to a straight or branched alkyl group in all isomeric forms. The term “C₍₁₋₅₎ alkyl” refers to an alkyl group, as defined above, containing at least 1, and at most 6 carbon atoms Examples of such alkyl groups include methyl, ethyl, propyl, iso-propyl: n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

As used herein, the term “halogen” refers to fluorine (F), chlorine (Cl), bromine (Br), or iodine (I) and the term “halo” refers to the halogen: fluoro (—F), chloro (—Cl), bromo(—Br) and iodo(—I).

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

Suitable compounds of formula (I) are:
4-{[4-(5-{3-Chloro-4-[(1-methylethyl)oxy]phenyl}-1,2,4-oxadiazol-3-yl)-3-methylphenyl]oxy}butanoic acid
5-{[4-(5-{3-Chloro-4-[(1-methylethyl)oxy]phenyl}-1,2,4-oxadiazol-3-yl)-3-methylphenyl]oxy}pentanoic acid
{[4-(5-{3-Chloro-4-[(1-methylethyl)oxy]phenyl}-1,2,4-oxadiazol-3-yl)-3-methylphenyl]oxy}acetic acid
3-{[4-(5-{3-Chloro-4-[(1-methylethyl)oxy]phenyl}-1,2,4-oxadiazol-3-yl)-3-methylphenyl]oxy}-N,N-dimethyl-1-propanamine
2-{[4-(5-{3-Chloro-4-[(1-methylethyl)oxy]phenyl}-1,2,4-oxadiazol-3-yl)-3-methylphenyl]oxy}-N-methylethanamine
5-{3-Chloro-4-[(1-methylethyl)oxy]phenyl}-3-(2-methyl-4-{[2-(methyloxy)ethyl]oxy}phenyl)-1,2,4-oxadiazole
5-{[3-Chloro-4-(5-{3-cyano-4-[(1-methylethyl)oxy]phenyl}-1,2,4-oxadiazol-3-yl)phenyl]oxy}pentanoic acid
5-{[4-(5-{3-Cyano-4-[(1-methylethyl)oxy]phenyl}-1,2,4-oxadiazol-3-yl)phenyl]oxy}pentanoic acid
{[3-Chloro-4-(5-{3-cyano-4-[(1-methylethyl)oxy]phenyl}-1,2,4-oxadiazol-3-yl)phenyl]oxy}acetic acid
4-{[3-Chloro-4-(5-{3-cyano-4-[(1-methylethyl)oxy]phenyl}-1,2,4-oxadiazol-3-yl)phenyl]oxy}butanoic acid
5-{[4-(5-{3-Cyano-4-[(1-methylethyl)oxy]phenyl}-1,2,4-oxadiazol-3-yl)-3-fluorophenyl]oxy}pentanoic acid
5-{[4-(5-{3-Cyano-4-[(1-methylethyl)oxy]phenyl}-1,2,4-oxadiazol-3-yl)-3-(methyloxy)phenyl]oxy}pentanoic acid
5-[5-(2-ethyl-4-{[3-(methylamino)propyl]oxy}phenyl)-1,3,4-thiadiazol-2-yl]-2-[(1-methylethyl)oxy]benzonitrile
5-[5-(2-ethyl-4-{[3-(methylamino)propyl]oxy}phenyl)-1,3,4-oxadiazol-2-yl]-2-[(1-methylethyl)oxy]benzonitrile
4-{[4-(5-{3-cyano-4-[(1-methylethyl)oxy]phenyl}-1,3,4-oxadiazol-2-yl)-3-ethylphenyl]oxy}butanoic acid
4-{[4-(5-{3-cyano-4-[(1-methylethyl)oxy]phenyl}-1,3,4-thiadiazol-2-yl)-3-ethylphenyl]oxy}butanoic acid
5-[3-(2-chloro-4-{[4-(dimethylamino)butyl]oxy}phenyl)-1,2,4-oxadiazol-5-yl]-2-[(1-methylethyl)oxy]benzonitrile
5-{3-[2-chloro-4-({4-[(1-methylethyl)amino]butyl}oxy)phenyl]-1,2,4-oxadiazol-5-yl}-2-[(1-methylethyl)oxy]benzonitrile
5-[3-(2-chloro-4-{[4-(propylamino)butyl]oxy}phenyl)-1,2,4-oxadiazol-5-yl]-2-[(1-methylethyl)oxy]benzonitrile
5-[3-(2-chloro-4-{[4-(ethylamino)butyl]oxy}phenyl)-1,2,4-oxadiazol-5-yl]-2-[(1-methylethyl)oxy]benzonitrile
5-[3-(2-chloro-4-{[4-(methylamino)butyl]oxy}phenyl)-1,2,4-oxadiazol-5-yl]-2-[(1-methylethyl)oxy]benzonitrile
4-{[4-(5-{3-cyano-4-[(1-methylethyl)oxy]phenyl}-1,2,4-oxadiazol-3-yl)-3-fluorophenyl]oxy}butanoic acid
{[4-(5-{3-cyano-4-[(1-methylethyl)oxy]phenyl}-1,2,4-oxadiazol-3-yl)-3-fluorophenyl]oxy}acetic acid
4-{[4-(5-{3-cyano-4-[(1-methylethyl)oxy]phenyl}-1,2,4-oxadiazol-3-yl)phenyl]oxy}butanoic acid
{[4-(5-{3-cyano-4-[(1-methylethyl)oxy]phenyl}-1,2,4-oxadiazol-3-yl)phenyl]oxy}acetic acid
(2-{[4-(5-{3-chloro-4-[(1-methylethyl)oxy]phenyl}-1,2,4-oxadiazol-3-yl)-3-ethylphenyl]oxy}ethyl)methylamine
{[4-(5-{3-cyano-4-[(1-methylethyl)oxy]phenyl}-1,2,4-oxadiazol-3-yl)-3-ethylphenyl]oxy}acetic acid
{[4-(5-{3-chloro-4-[(1-methylethyl)oxy]phenyl}-1,2,4-oxadiazol-3-yl)-3-ethylphenyl]oxy}acetic acid
5-{[4-(5-{3-chloro-4-[(1-methylethyl)oxy]phenyl}-1,2,4-oxadiazol-3-yl)-3-ethylphenyl]oxy}pentanoic acid
4-{[4-(5-{3-chloro-4-[(1-methylethyl)oxy]phenyl}-1,2,4-oxadiazol-3-yl)-3-ethylphenyl]oxy}butanoic acid
5-{[4-(5-{3-cyano-4-[(1-methylethyl)oxy]phenyl}-1,2,4-oxadiazol-3-yl)-3-ethylphenyl]oxy}pentanoic acid
4-{[4-(5-{3-cyano-4-[(1-methylethyl)oxy]phenyl}-1,2,4-oxadiazol-3-yl)-3-ethylphenyl]oxy}butanoic acid
5-[3-(2-ethyl-4-{[2-(methylamino)ethyl]oxy}phenyl)-1,2,4-oxadiazol-5-yl]-2-[(1-methylethyl)oxy]benzonitrile
(4-{[4-(5-{3-chloro-4-[(1-methylethyl)oxy]phenyl}-1,2,4-oxadiazol-3-yl)-3-ethylphenyl]oxy}butyl)methylamine
(3-{[4-(5-{3-chloro-4-[(1-methylethyl)oxy]phenyl}-1,2,4-oxadiazol-3-yl)-3-ethylphenyl]oxy}propyl)methylamine
5-[3-(2-chloro-4-{[2-(methylamino)ethyl]oxy}phenyl)-1,2,4-oxadiazol-5-yl]-2-[(1-methylethyl)oxy]benzonitrile
5-[3-(2-chloro-4-{[3-(methylamino)propyl]oxy}phenyl)-1,2,4-oxadiazol-5-yl]-2-[(1-methylethyl)oxy]benzonitrile
5-[3-(2-ethyl-4-{[4-(methylamino)butyl]oxy}phenyl)-1,2,4-oxadiazol-5-yl]-2-[(1-methylethyl)oxy]benzonitrile
5-[3-(2-ethyl-4-{[3-(nnethylamino)propyl]oxy}phenyl)-1,2,4-oxadiazol-5-yl]-2-[(1-methylethyl)oxy]benzonitrile
(3-{[4-(5-{3-chloro-4-[(1-methylethyl)oxy]phenyl}-1,2,4-oxadiazol-3-yl)-3-methylphenyl]oxy}propyl)methylamine
4-{[4-(3-{3-cyano-4-[(1-methylethyl)oxy]phenyl}-1,2,4-thiadiazol-5-yl)-3-ethylphenyl]oxy}butanoic acid
4-{[4-(5-{3-cyano-4-[(1-methylethyl)oxy]phenyl}-1,2,4-thiadiazol-3-yl)-3-ethylphenyl]oxy}butanoic acid
4-{[4-(2-{3-cyano-4-[(1-methylethyl)oxy]phenyl}-1,3-thiazol-5-yl)-3-ethylphenyl]oxy}butanoic acid
5-[5-(2-ethyl-4-{[3-(methylamino)propyl]oxy}phenyl)-1,3-thiazol-2-yl]-2-[(1-methylethyl)oxy]benzonitrile or salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolic or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

In a further aspect, this invention provides processes for preparation of a compound of formula (I). In one aspect, certain compounds of formula (I) were prepared by the process in Schemes I to IX.

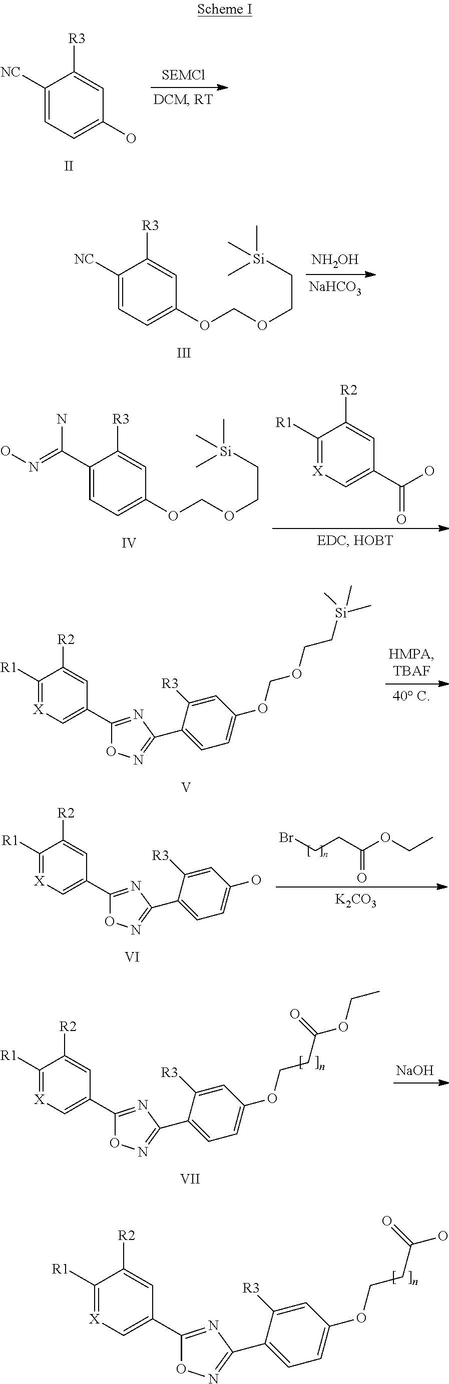

The first step of the process (II to III) is carried out in DCM at room temperature. In the second step of the process (III to IV) suitable reagents include NH₂OH and NaHCO₃. In the third step of coupling (IV to V) suitable reagents include EDCl, HOBT in THF and dioxane. Followed by the de-SEM process (V to VI). The fifth step of the process (VI to VII) suitable reagents include bromide and K₂CO₃ in a solvent such as DMF at 60° C. The last step of the process (VII to I) is carried out by treatment with basic (such as sodium hydroxide in a suitable solvent such as isopropanol) conditions and may be carried out at room temperature or 90° C.

In another aspect, compounds of formula (I) can be prepared by the process in Scheme II to IX.

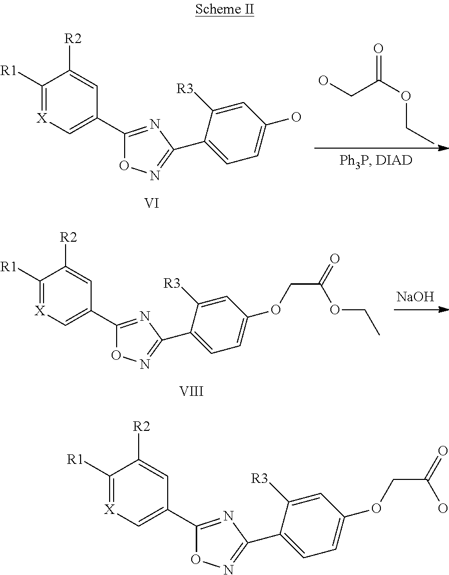

The first step of the process (VI to VIII) suitable reagents include bromide and Ph₃P and DIAD in a solvent such as THF at room temperature. The last step of the process (VIII to I) is carried out by treatment with basic (such as sodium hydroxide in a suitable solvent such as isopropanol) conditions and may be carried out at room temperature or 90° C.

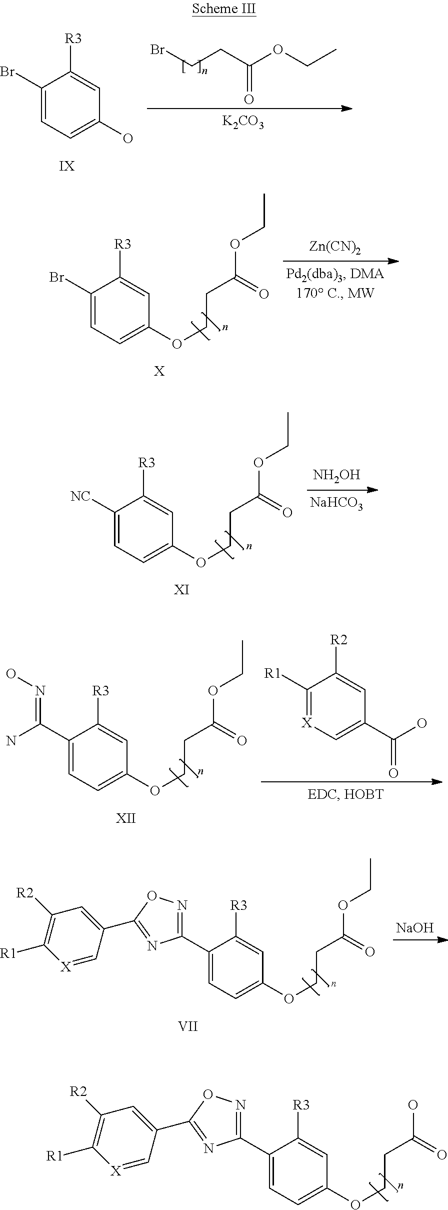

The first step of the process (IX to X) suitable reagents include bromide and K₂CD₃ in a solvent such as DMF at 45° C. The second step of process (X to XI) is carried out under microwave condition. The followed two steps of process (XI to VII) are similar with process (III to V) in scheme I. The last step of process (VII to I) is carried out by treatment with basic (such as sodium hydroxide in a suitable solvent such as isopropanol) conditions and may be carried out at room temperature or 90° C.

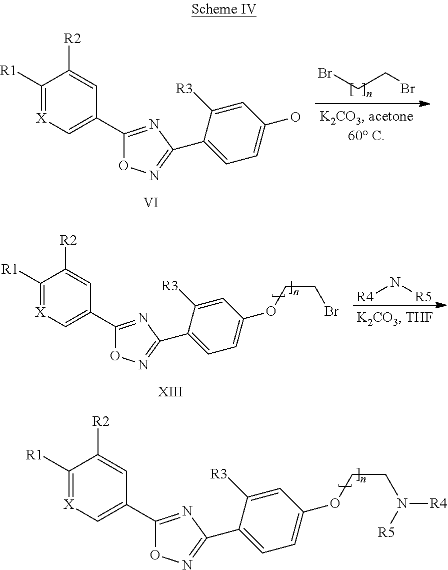

The first step of the process (VI to XIII) suitable reagents include bromide and K₂CO₃ in a solvent such as acetone at 60° C. The last step of process (XIII to I) is carried out with the suitable reagents such as amine and K₂CO₃ in a suitable solvent such as THF.

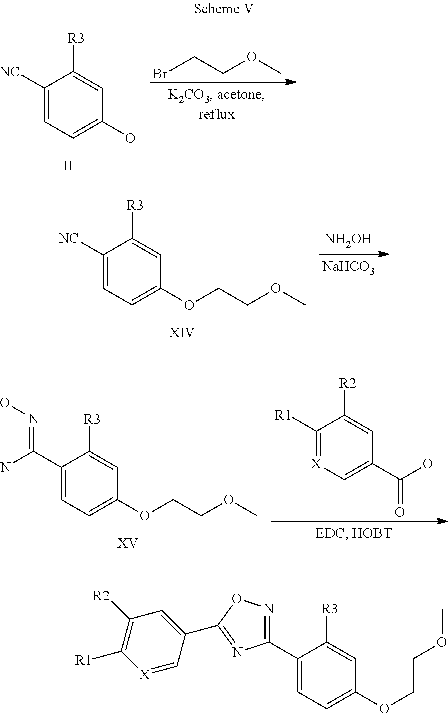

The first step of the process (II to XIV) suitable reagents include bromide and K₂CO₃ in a solvent such as acetone at 60° C. The last two steps of process (XIV to I) is similar with the process (XI to VII) in scheme III.

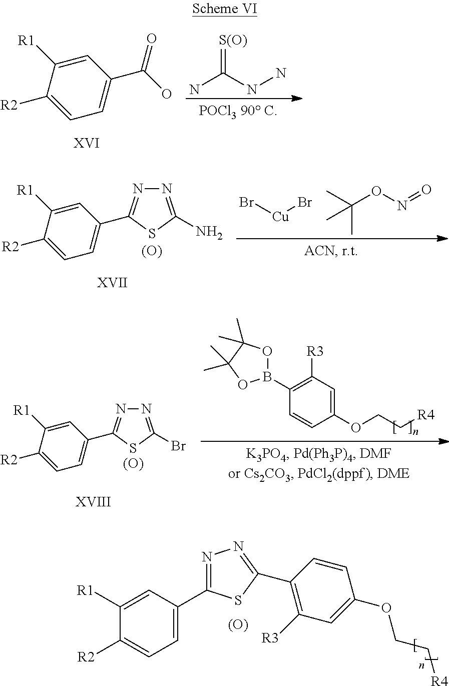

The first step of the process (XVI to XVII) is carried out in POCl₃ at 90° C. In the second step of the process (XVII to XVIII) suitable reagents include CuBr₂ and 1,1-dimethylethyl nitrite at 0° C. In the third step of Suzuki coupling process(XVIII to I) suitable reagents include Pd(PPh₃)₄ and K₃PO₄ or Cs₂CO₃ and PdCl₂(dppf) in a solvent such as DMF or DME under microwave condition, followed by hydrolysis or de-protection process.

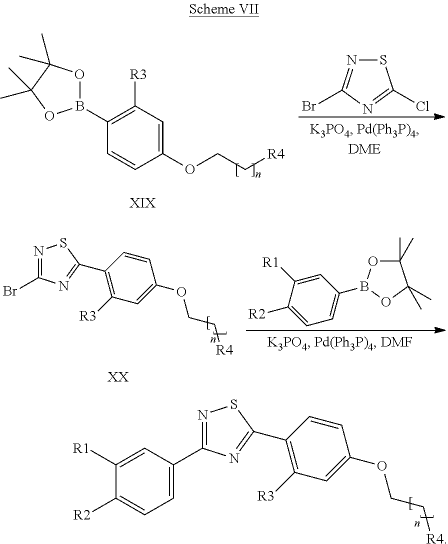

The two steps of Suzuki coupling process (XIX to I) are carried out with suitable reagents include Pd(PPh₃)₄ and K₃PO₄ and in a solvent such as DMF or DME under microwave condition, followed by hydrolysis or de-protection process.

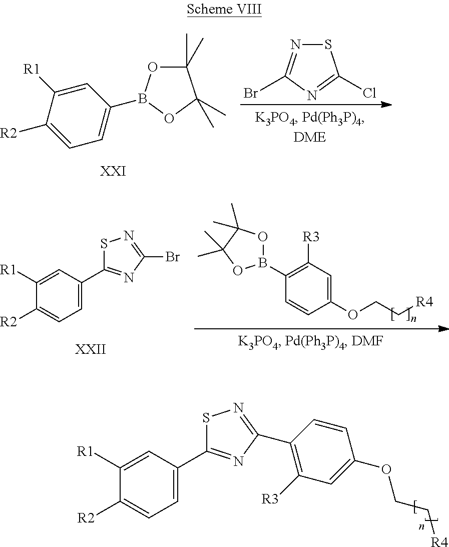

The two steps of Suzuki coupling process (XXI to I) are similar with process (XIX to I) in scheme VII.

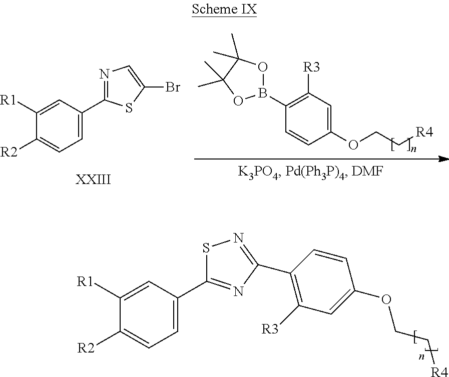

This step of process (XXIII to I) is carried out with suitable reagents include Pd(PPh₃)₄ and K₃PO₄ and in a solvent such as DMF or DME under microwave condition, followed by hydrolysis or de-protection process.

Pharmaceutically acceptable salts may be prepared conventionally by reaction with the appropriate acid or acid derivative.

The potencies and efficacies of the compounds of this invention for the S1P1 receptor can be determined by GTPγS assay or S1P1 Tango assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides a compound of formula (I) or a pharmaceutically acceptable salt thereof, for use as a therapeutic substance, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes. The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof.

Compounds of formula (I) and their pharmaceutically acceptable salts are of use as therapeutic substances in the treatment of multiple sclerosis.

In another aspect, the invention provides for the use of a compound of formula (I) or a pharmaceutically acceptable salt thereof in the manufacture of a medicament for use in the treatment of the conditions or disorders mediated via the S1P1 receptor

The invention provides a method of treatment of multiple sclerosis, which comprises administering to the sufferer a therapeutically safe and effective amount of a compound of formula (I) or a pharmaceutically acceptable salt thereof.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same mariner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations. For example, the compounds of the invention may be used in combination with cyclosporin A, methotrexate, steroids, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable saltss of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### ABBREVIATIONS

g—grams
mg—milligrams
ml—millilitres
min—minute
ul—microlitres
MeCN—acetonitrile
MeOH—methanol
EtOH—ethanol
Et₂O—diethyl ether
EtOAc—ethyl acetate
DABCO—1,4-diazabiclo[2,2,2]octane
DCM—dichloromethane
DIAD—diisopropyl azodicarboxylate
DME—1,2-bis(methyloxy)ethane
DMF—N,N-dimethylformamide
DMSO—dimethylsulphoxide
EDAC—N-(3-Dimethylaminopropyl)-N′-ethylcarbodiimide hydrochloride
EDC—N-(3-Dimethylaminopropyl)-N′-ethylcarbodiimide hydrochloride
EDCl—N-(3-Dimethylaminopropyl)-N′-ethylcarbodiimide hydrochloride
HOBT/HOBt—Hydroxybenzotriazole
IPA—isopropylalcohol
NCS—N-chlorosuccinimide
PyBOP—Benzotriazol-1-yl-oxytripyrrolidinophosphonium hexafluorophosphate
THF—tetrahydrofuran
dba—dibenzylidene acetone
RT—room temperature
° C.—degrees Celsius
M—Molar
H—proton
s—singlet
d—doublet
t—triplet
q—quartet
MHz—megahertz
MeOD—deuterated methanol
LCMS—Liquid Chromatography Mass Spectrometry
LC/MS—Liquid Chromatography Mass Spectrometry
MS—mass spectrometry
ES—Electrospray
MH⁺—mass ion+H⁺
MDAP—mass directed automated preparative liquid chromatography.
sat.—saturated

### Chromatography

Unless stated otherwise, all chromatography was carried out using silica columns.

### General Chemistry Section

The intermediates for the preparation of the examples may not necessarily have been prepared from the specific batch of precursor described.

### Description for D1

### 2-Methyl-4-[({[2-(trimethylsilyl)-ethyl]oxy}methyl)oxy]benzonitrile (D1)

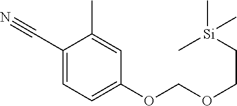

To the solution of 4-hydroxy-2-methylbenzonitrile (1 g) and DIPEA (2.62 mL) in dichloromethane (100 mL) was added SEMCl (1.399 mL) at 25° C. dropwise. The reaction solution was stirred for 2 hours. Solvent was removed in vacuo and the residue was purified by column to afford 2-methyl-4-[({[2-(trimethylsilyl)ethyl]oxy}methyl)oxy]benzonitrile (D1) (1.65 g) as a colorless oil. δH (CDCl₃, 400 MHz): 0.01 (9H, s), 0.95 (2H, t), 2.52 (3H, s), 3.75 (2H, m), 5.25 (2H, s), 6.92 (1H, dd), 6.96 (1H, d), 7.52 (1H, d). MS (ES): C₁₄H₂₁H₂₁NO₂Si requires 263; found 264.2 (M+H⁺).

### Description for D2

### N-hydroxy-2-methyl-4-[({[2-(trimethylsilyl)ethyl]oxy}methyl)oxy]benzenecarboximidamide (D2)

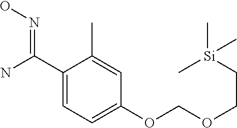

To the suspension of 2-methyl-4-[({[2-(trimethylsilyl)ethyl]oxy}methyl)oxy]benzonitrile (D1) (1.65 g) and NaHCO₃, (6.31 g) in EtOH (70 mL) was added hydroxylamine hydrochloride (4.35 g). The mixture was refluxed for two days. The solvent was removed in vacuo and the residue was dissolved in ethyl acetate (100 mL), washed with water (2×15 mL), dried over sodium sulphate, concentrated to afford N-hydroxy-2-methyl-4-[({[2-(trimethylsilyl)ethyl]oxy}methyl)oxy]benzenecarboximida mide (D2) (1.85 g) as a clear oil. MS (ES): C₁₄H₂₄N₂O₃Si requires 296; found 297.2 (M+H⁺).

### Description for D3

### 5-{3-Chloro-4-[(1-methylethyl)oxy]phenyl}-3-{2-methyl-4-[({[2-(trimethylsilyl)ethyl]oxy}methyl)oxy]phenyl}-1,2,4-oxadiazole (D3)

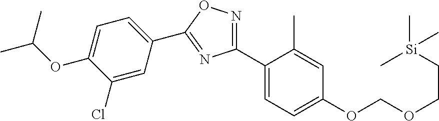

To the solution of 3-chloro-4-[(1-methylethyl)oxy]benzoic acid (1.41 g) in THF (70 mL) was added EDC (1.93 g), HOBt (1.54 g) and the resulting solution was stirred at RT for 2 hours. To the reaction solution was added N-hydroxy-2-methyl-4-[({[2-(trimethylsilyl)ethyl]oxy}methyl)oxy]benzenecarboximidamide (D2) (1.85 g) and the suspension was stirred at RT for another 2 hours. THF was removed in vacuo and the residue was dissolved in ethyl acetate (80 mL), washed with water (2×15 mL), dried over sodium sulphate, concentrated. The residue in dioxane (60 mL) was heated to reflux for 5 hours. Dioxane was removed in vacuo and the residue was purified by column to afford 5-{3-chloro-4-[(1-methylethyl)oxy]phenyl}-3-{2-methyl-4-[({[2-(trimethylsilyl)ethyl]oxy}methyl)oxy]phenyl}-1,2,4-oxadiazole (D3) (1.7 g). δH (CDCl₃, 400 MHz): 0.02 (9H, s), 0.98 (2H, t), 1.46 (6H, d), 2.67 (3H, s), 3.79 (2H, t), 4.72 (1H, m), 5.29 (2H, s), 7.01 (2H, m), 7.06 (1H, d), 8.05 (2H, m), 8.24 (1H, d). MS (ES): C₂₄H₃₁ClN₂O₄Si requires 474; found 475.2 (M+H⁺).

### Description for D4

### Ethyl 4-{[4-(5-{3-chloro-4-[(1-methylethyl)oxy]phenyl}-1,2,4-oxadiazol-3-yl)-3-methylphenyl]oxy}butanoate (D4)

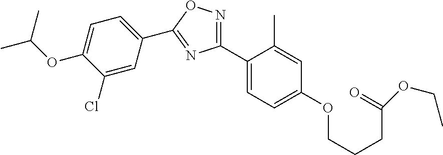

The mixture of 4-(5-{3-chloro-4-[(1-methylethyl)oxy]phenyl}-1,2,4-oxadiazol-3-yl)-3-methylphenol (100 mg), ethyl 4-bromobutanoate (0.083 mL) and potassium carbonate (120 mg) in acetone (5 mL) was heated to 60° C. for overnight. The solvent was removed in vacuo and the residue was dissolved in ethyl acetate (20 mL), washed with water, dried over sodium sulphate. After concentration and the residue was purified by column to afford ethyl 4-{[4-(5-{3-chloro-4-[(1-methylethyl)oxy]phenyl}-1,2,4-oxadiazol-3-yl)-3-methylphenyl]oxy}butanoate (D4) (0.1 g) as a clear oil. MS (ES): C₂₄H₂₇ClN₂O₅ requires 458; found 459.2 (M+H⁺).

### Description for D5

### Ethyl 5-{[4-(5-{3-chloro-4-[(1-methylethyl)oxy]phenyl}-1,2,4-oxadiazol-3-yl)-3-methylphenyl]oxy}pentanoate (D5)

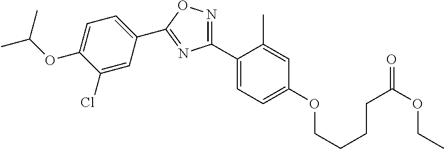

The mixture of 4-(5-{3-chloro-4-[(1-methylethyl)oxy]phenyl}-1,2,4-oxadiazol-3-yl)-3-methylphenol (80 mg), ethyl 5-bromopentanoate (97 mg) and potassium carbonate (96 mg) in acetone (3 mL) was heated to 60° C. for overnight. The solvent was removed in vacuo and the residue was dissolved in ethyl acetate (20 mL), washed with water, dried over sodium sulphate. After concentration and the residue was purified by column to afford ethyl 5-{[4-(5-{3-chloro-4-[(1-methylethyl)oxy]phenyl}-1,2,4-oxadiazol-3-yl)-3-methylphenyl]oxy}pentanoate (D5) (0.1 g) as a clear oil. MS (ES): C₂₅H₂₉ClN₂O₅ requires 472; found 473.2 (M+H⁺).

### Description for D6

### Ethyl {[4-(5-{3-chloro-4-[(1-methylethyl)oxy]phenyl}-1,2,4-oxadiazol-3-yl)-3-methylphenyl]oxy}acetate (D6)

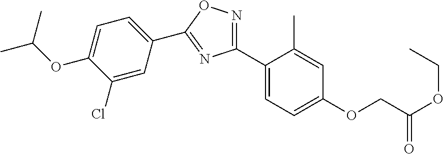

The mixture of 4-(5-{3-chloro-4-[(1-methylethyl)oxy]phenyl}-1,2,4-oxadiazol-3-yl)-3-methylphenol (80 mg), Ph₃P (183 mg) and ethyl hydroxyacetate (0.044 mL) in tetrahydrofuran (5 mL) was added DIAD (0.135 mL) dropwise under nitrogen atmosphere at room temp. The solution was heated to 70° C. for overnight. The solvent was removed and the residue was dissolved in ethyl acetate (20 mL), washed with water (10 mL), dried over sodium sulphate, concentrated and purified by column to afford ethyl {[4-(5-{3-chloro-4-[(1-methylethyl)oxy]phenyl}-1,2,4-oxadiazol-3-yl)-3-methylphenyl]oxy}acetate (D6) (100 mg) as a clear oil. MS (ES): C₂₂H₂₃ClN₂O₅ requires 430; found 431.2 (M+H⁺).

### Description for D7

### 3-{4-[(2-Bromoethyl)oxy]-2-methylphenyl}-5-{3-chloro-4-[(1-methylethyl)oxy]phenyl}-1,2,4-oxadiazole (D7)

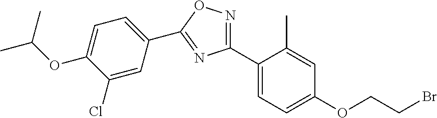

To the suspension of 4-(5-{3-chloro-4-[(1-methylethyl)oxy]phenyl}-1,2,4-oxadiazol-3-yl)-3-methylphenol (80 mg) and 1,2-dibromoethane (0.400 mL) in acetone (3 mL) was added potassium carbonate (96 mg). The resultiong suspension was heated at 60° C. for overnight. The solvent was removed in vacuo and the residue was purified by column to afford 3-{4-[(2-bromoethyl)oxy]-2-methylphenyl}-5-{3-chloro-4-[(1-methylethyl)oxy]phenyl}-1,2,4-oxadiazole (D7) (50 mg) as a white solid. δH (CDCl₃, 400 MHz): 1.46 (6H, d), 2.67 (3H, s), 3.68 (2H, m), 4.36 (2H, m), 4.72 (1H, m), 6.87 (2H, m), 7.06 (1H, d), 8.06 (2H, m), 8.24 (1H, d). MS (ES): C₂₀H₂₀BrClN₂O₃ requires 450; found 451.1 (M+H⁺).

### Description for D8

### 2-Methyl-4-{[2-(methyloxy)ethyl]oxy}benzonitrile (D8)

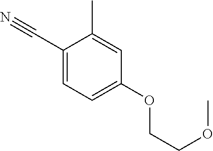

The mixture of 4-hydroxy-2-methylbenzonitrile (100 mg), 2-bromoethyl methyl ether (209 mg) and potassium carbonate (311 mg) in acetone (8 mL) was heated to reflux for two days. The solid was filtered and the liquor was concentrated, purified by column to afford 2-methyl-4-{[2-(methyloxy)ethyl]oxy}benzonitrile (D8) (100 mg). δH (CDCl₃, 400 MHz): 2.51 (3H, s), 3.46 (3H, s), 3.76 (2H, t), 4.15 (2H, t), 6.80 (1H, dd), 6.84 (1H, d), 7.52 (1H, d). MS (ES): C₁₁H₁₃NO₂ requires 191; found 192.1 (M+H⁺).

### Description for D9

### N-hydroxy-2-methyl-4-{[2-(methyloxy)ethyl]oxy}benzenecarboximidamide (D9)

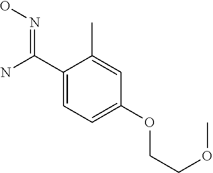

To the suspension of 2-methyl-4-{[2-(methyloxy)ethyl]oxy}benzonitrile (D8) (100 mg) and NaHCO₃, (308 mg) in EtOH (10 mL) was added hydroxylamine hydrochloride (182 mg). The mixture was refluxed for two days. The solvent was removed in vacuo and the residue was dissolved in ethyl acetate (50 mL), washed with water (2×7 mL), dried over sodium sulphate, concentrated to afford N-hydroxy-2-methyl-4-{([2-(methyloxy)ethyl]oxy}benzenecarboximidamide (D9) (117 mg) as a crude product. MS (ES): C₁₁H₁₆N₂O₃ requires 224; found 225.2 (M+H⁺).

### Description for D10

### 2-Chloro-4-[({[2-(trimethylsilyi)ethyl]oxy}methyl)oxy]benzonitrile (D10)

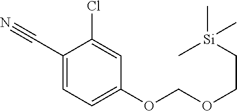

To the solution of 2-chloro-4-hydroxybenzonitrile (2 g) and DIPEA (1.85 g) in dichloromethane (200 mL) was added SEMCl (2.17 g) at 25° C. dropwise. The reaction solution was sitrred for 2 hours. The reaction solution was washed with water (2×50 ml), dried over sodium sulphate, concentrated to afford the crude product 2-chloro-4-[({[2-(trimethylsilyl)ethyl]oxy}methyl)oxy]benzonitrile (D10) (4.07 g) as a grey oil. δH (CDCl₃, 400 MHz): 0.01 (9H, s), 0.95 (2H, t), 3.75 (2H, t), 5.27 (2H, s), 7.01 (1H, dd), 7.19 (1H, d), 7.58 (1H, d). MS (ES): C₁₃H₁₈ClNO₂Si requires 283; found 284.1 (M+H⁺).

### Description for D11

### 2-Chloro-N-hydroxy-4-[({[2-(trimethylsilyl)ethy]oxy}methyl)oxy]benzenecarboximidamide (D11)

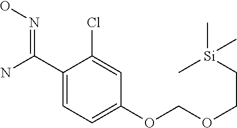

To the suspension of 2-chloro-4-[({[2-(trimethylsilyl)ethyl]oxy}methyl)oxy]benzonitrile (D10) (4.07 g) and NaHCO₃, (8.43 g) in EtOH (100 mL) was added hydroxylamine hydrochloride (4.98 g). The mixture was refluxed for 36 hours. The solvent was removed in vacuo and the residue was dissolved in ethyl acetate (150 mL), washed with water (2×20 mL), dried over sodium sulphate, concentrated to afford 2-chloro-N-hydroxy-4-[({[2-(trimethylsilyl)ethyl]oxy}methyl)oxy]benzenecarboximidamide (D11) (4.54 g) as a crude product. MS (ES): C₁₃H₂₁ClN₂O₃Si requires 316; found 317.2 (M+H⁺).

### Description for D12

### 5-(3-{2-Chloro-4-[({[2-(trimethylsilyl)ethyl]oxy}methyl)oxy]phenyl}-1,2,4-oxadiazol-5-yl)-2-[(1-methylethyl)oxy]benzonitrile (D12)

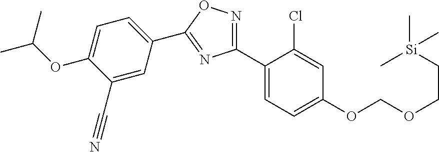

To the solution of 3-cyano-4-[(1-methylethyl)oxy]benzoic acid (2.94 g) in THF (100 mL) was added EDC (4.12 g), HOBt (3.29 g) and the resulting solution was stirred at RT for 2 hours. To the reaction solution was added 2-chloro-N-hydroxy-4-[({[2-(trimethylsilyl)ethyl]oxy}methyl)oxy]benzenecarboximidamide (D11) (4.54 g) and the suspension was stirred at RT for another 2 hours. THF was removed in vacuo and the residue was dissolved in ethyl acetate (150 mL), washed with water (2×20 mL), dried over sodium sulphate, concentrated. The residue in dioxane (60 mL) was heated to reflux for 5 hours. Dioxane was removed in vacuo and the residue was purified by column to afford 5-(3-{2-chloro-4-[({[2-(trimethylsilyl)ethyl]oxy}methyl)oxy]phenyl}-1,2,4-oxadiazol-5-yl)-2-[(1-methylethyl)oxy]benzonitrile (D12) (4.05 g). δH (CDCl₃, 400 MHz): 0.02 (9H, s), 0.98 (2H, t), 1.48 (6H, d), 3.78 (2H, m), 4.80 (1H, m), 5.29 (2H, s), 7.11 (2H, m), 7.26 (1H, d), 7.97 (1H, d), 8.34 (1H, dd), 8.43 (1H, d). MS (ES): C₂₄H₂₈ClN₃O₄Si requires 485; found 486.2 (M+H⁺).

### Description for D13

### 5-[3-(2-Chloro-4-hydroxyphenyl)-1,2,4-oxadiazol-5-yl]-2-[(1-methylethyl)oxy]benzonitrile (D13)

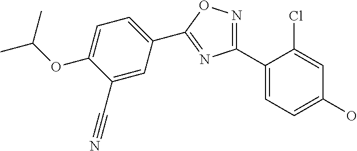

To the solution of 5-(3-{2-chloro-4-[({[2-(trimethylsilyl)ethyl]oxy}methyl)oxy]phenyl}-1,2,4-oxadiazol-5-yl)-2-[(1-methylethyl)oxy]benzonitrile (D12) (4.05 g) in HMPA (8 mL) was added TBAF (6.54 g) at room temp. The resulting solution was heated to 40° C. for overnight. The reaction solution was poured into water (100 mL), extracted with ethyl acetate (3×40 ml) and the combined organic phases was washed with water (30 mL), dried over sodium sulphate, concentrated and purified by column to afford 5-[3-(2-chloro-4-hydroxyphenyl)-1,2,4-oxadiazol-5-yl]-2-[(1-methylethyl)oxy]benzonitrile (D13) (2.2 g) as a light brown solid. δH (CDCl₃, 400 MHz): 1.49 (6H, d), 4.81 (1H, m), 6.90 (1H, dd), 7.07 (1H, d), 7.13 (1H, d), 7.95 (1H, d), 8.33 (1H, dd), 8.43 (1H, d). MS (ES): C₁₈H₁₄ClN₃O₃ requires 355; found 356.1 (M+H⁺).

### Description for D14

### Ethyl 5-{[3-chloro-4-(5-{3-cyano-4-[(1-methylethyl)oxy]phenyl}-1,2,4-oxadiazol-3-yl)phenyl]oxy}pentanoate (D14)

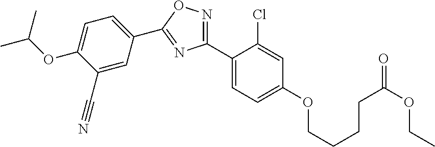

The mixture of 5-[3-(2-chloro-4-hydroxyphenyl)-1,2,4-oxadiazol-5-yl]-2-[(1-methylethyl)oxy]benzonitrile (D13) (120 mg), ethyl 5-bromopentanoate (212 mg) and potassium carbonate (233 mg) in DMF (3 mL) was heated to 60° C. for overnight, he reaction suspension was poured into water (5 mL), extracted with ethyl acetate (2×10 mL) and the combined organic phases were dried over sodium sulphate, concentrated and purified by column to afford ethyl 5-{[3-chloro-4-(5-{3-cyano-4-[(1-methylethyl)oxy]phenyl}-1,2,4-oxadiazol-3-yl)phenyl]oxy}pentanoate (D14) (140 mg) as a pale white solid. MS (ES): C₂₅H₂₆ClN₃O₅ requires 483; found 484.2 (M+H⁺).

### Description for D15

### 4-[({[2-(Trimethylsilyl)-ethyl]oxy}methyl)oxy]benzonitrile (D15)

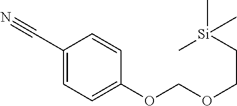

To the solution of 4-hydroxybenzonitrile (2 g) and DIPEA (2.39 g) in dichloromethane (200 mL) was added SEMCl (2.80 g) at 25° C. dropwise. The reaction solution was sitrred for 2 hours. The reaction solution was washed with water (2×50 mL), dried over sodium sulphate, concentrated to afford the crude product 4-[({[2-(trimethylsilyl)ethyl]oxy}methyl)oxy]benzonitrile (D15) (4.19 g) as a clear oil. MS (ES): C₁₃H₁₉NO₂Si requires 249; found 250.1 (M+H⁺).

### Description for D16

### N-hydroxy-4-[({[2-(trimethylsilyl)ethyl]oxy}methyl)oxy]benzenecarboximidamide (D16)

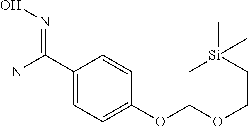

To the suspension of 4-[({[2-(trimethylsilyl)ethyl]oxy}methyl)oxy]benzonitrile (D15) (4.19 g) and NaHCO₃, (9.90 g) in EtOH (150 mL) was added hydroxylamine hydrochloride (5.85 g). The mixture was refluxed for two days. The solvent was removed in vacuo and the residue was dissolved in ethyl acetate (150 mL), washed with water (2×20 mL), dried over sodium sulphate, concentrated to afford N-hydroxy-4-[({[2-(trimethylsilyl)ethyl]oxy}methyl)oxy]benzenecarboximidamide (D16) (4.76 g) as a crude product. MS (ES): C₁₃H₂₂N₂O₃Si requires 282; found 283.2 (M+H⁺).

### Description for D17

### 2-[(1-Methylethyl)oxy]-5-(3-{4-[({[2-(trimethylsilyi)ethyl]oxy}methyl)oxy]phenyl}-1,2,4-oxadiazol-5-yl)benzonitrile (D17)

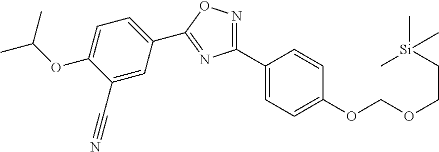

To the solution of 3-cyano-4-[(1-methylethyl)oxy]benzoic acid (3.46 g) in THF (200 mL) was added EDC (4.85 g), HOBt (3.87 g) and the resulting solution was stirred at RT for 2 hours. To the reaction solution was added N-hydroxy-4-[({[2-(trimethylsilyl)ethyl]oxy}methyl)oxy]benzenecarboximidamide (D16) (4.76 g) and the suspension was stirred at RT for another 2 hours. THF was removed in vacuo and the residue was dissolved in ethyl acetate (150 mL), washed with water (2×20 mL), dried over sodium sulphate, concentrated. The residue in dioxane (100 mL) was heated to reflux for overnight. Dioxane was removed in vacuo and the residue was purified by column to afford 2-[(1-methylethyl)oxy]-5-(3-{4-[({[2-(trimethylsilyl)ethyl]oxy}methyl)oxy]phenyl}-1,2,4-oxadiazol-5-yl)benzonitrile (D17) (6.2 g) as a solid. δH (CDCl₃, 400 MHz): 0.02 (9H, s), 0.98 (2H, t), 1.48 (6H, d), 3.79 (2H, t), 4.80 (1H, m), 5.31 (2H, s), 7.12 (1H, d), 7.16 (2H, d), 8.09 (2H, d), 8.33 (1H, dd), 8.43 (1H, d). MS (ES): C₂₄H₂₉N₃O₄Si requires 451; found 452.2 (M+H⁺).

### Description for D18

### 5-[3-(4-Hydroxyphenyl)-1,2,4-oxadiazol-5-yl]-2-[(1-methylethyl)oxy]benzonitrile (D18)

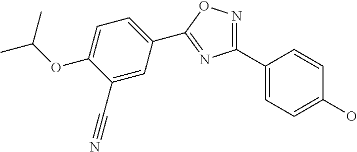

To the solution of 2-[(1-methylethyl)oxy]-5-(3-{4-[({[2-(trimethylsilyl)ethyl]oxy}methyl)oxy]phenyl}-1,2,4-oxadiazol-5-yl)benzonitrile (D17) (6.2 g) in HMPA (10 mL) was added TBAF (10.77 g) at room temp. The resulting solution was heated to 40° C. for overnight. The reaction solution was poured into water (150 mL), extracted with ethyl acetate (3×40 mL) and the combined organic phases was washed with water (30 mL), dried over sodium sulphate, concentrated and purified by column to afford 5-[3-(4-hydroxyphenyl)-1,2,4-oxadiazol-5-yl]-2-[(1-methylethyl)oxy]benzonitrile (D18) (3.8 g). δH (DMSO-d₆, 400 MHz): 1.37 (6H, d), 4.96 (1H, m), 6.93 (2H, d), 7.53 (1H, d), 7.90 (2H, d), 8.37 (1H, dd), 8.46 (1H, d), 10.17 (1H, s). MS (ES): C₁₈H₁₅N₃O₃ requires 321; found 322.2 (M+H⁺).

### Description for D19

Ethyl 5-{[4-(5-{3-cyano-4-[(1-methylethyl)oxy]phenyl}-1,2,4-oxadiazol-3-yl)phenyl]oxy}pentanoate (D19)

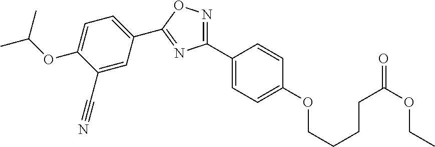

The mixture of 5-[3-(4-hydroxyphenyl)-1,2,4-oxadiazol-5-yl]-2-[(1-methylethyl)oxy]benzonitrile (D18) (120 mg), ethyl 5-bromopentanoate (234 mg) and potassium carbonate (258 mg) in DMF (3 mL) was heated to 60° C. for overnight. he reaction suspension was poured into water (5 mL), extracted with ethyl acetate (2×10 mL) and the combined organic phases were dried over sodium sulphate, concentrated and purified by column to afford ethyl 5-{[4-(5-{3-cyano-4-[(1-methylethyl)oxy]phenyl}-1,2,4-oxadiazol-3-yl)phenyl]oxy}pentanoate (D19) (160 mg) as a pale white solid. δH (CDCl₃, 400 MHz): 1.28 (3H, t), 1.48 (6H, d), 1.87 (4H, m), 2.42 (2H, t), 4.07 (2H, t), 4.14 (2H, q), 4.80 (1H, m), 7.01 (2H, d), 7.12 (1H, d), 8.08 (2H, d), 8.34 (1H, dd), 8.43 (1H, d). MS (ES): C₂₅H₂₇N₃O₅ requires 449; found 450.2 (M+H⁺).

### Description for D20

### Ethyl {[3-chloro-4-(5-{3-cyano-4-[(1-methylethyl)oxy]phenyl}-1,2,4-oxadiazol-3-yl)phenyl]oxy}acetate (D20)

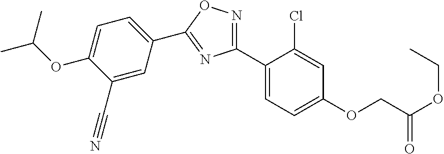

The mixture of 5-[3-(2-chloro-4-hydroxyphenyl)-1,2,4-oxadiazol-5-yl]-2-[(1-methylethyl)oxy]benzonitrile (D13) (120 mg), ethyl bromoacetate (169 mg) and potassium carbonate (233 mg) in DMF (3 mL) was heated to 60° C. for overnight, he reaction suspension was poured into water (5 mL), extracted with ethyl acetate (2×10 mL) and the combined organic phases were dried over sodium sulphate, concentrated and purified by column to afford ethyl {[3-chloro-4-(5-{3-cyano-4-[(1-methylethyl)oxy]phenyl}-1,2,4-oxadiazol-3-yl)phenyl]oxy}acetate (D20) (90 mg) as a pale white solid. MS (ES): C₂₂H₂₀ClN₃O₅ requires 441; found 442.2 (M+H⁺).

### Description for D21

### Ethyl 4-{[3-chloro-4-(5-{3-cyano-4-[(1-methylethyl)oxy]phenyl}-1,2,4-oxadiazol-3-yl)phenyl]oxy}butanoate (D21)

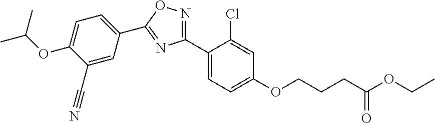

The mixture of 5-[3-(2-chloro-4-hydroxyphenyl)-1,2,4-oxadiazol-5-yl]-2-[(1-methylethyl)oxy]benzonitrile (D13) (120 mg), ethyl 4-bromobutanoate (197 mg) and potassium carbonate (233 mg) in acetone (5 mL) was heated to 60° C. for overnight. The solvent was removed in vacuo and the residue was dissolved in ethyl acetate (20 mL), washed with water, dried over sodium sulphate. After concentration and the residue was purified by column to afford ethyl 4-{[3-chloro-4-(5-{3-cyano-4-[(1-methylethyl)oxy]phenyl}-1,2,4-oxadiazol-3-yl)phenyl]oxy}butanoate (D21) (157 mg) as a solid. MS (ES): C₂₄H₂₄ClN₃O₅ requires 469; found 470.2 (M+H⁺).

### Description for D22

### 2-Fluoro-4-[({[2-(trimethylsilyl)ethyl]oxy}methyl)oxy]benzonitrile (D22)

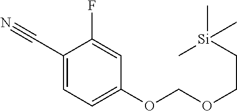

To the solution of 2-fluoro-4-hydroxybenzonitrile (2 g) and DIPEA (2.07 mL) in dichloromethane (100 mL) was added SEMCl (2.43 g) at 25° C. dropwise. The reaction solution was sitrred for 2 hours. The reaction solution was washed with water (2×15 mL), dried over sodium sulphate, concentrated to afford 2-fluoro-4-[({[2-(trimethylsilyl)ethyl]oxy}methyl)oxy]benzonitrile (D22) (3.90 g) as a clear oil. MS (ES): C₁₄H₂₁NO₂Si requires 263; found 264.2 (M+H⁺).

### Description for D23

### 2-Fluoro-N-hydroxy-4-[({[2-(trimethylsilyi)ethyl]oxy}methyl)oxy]benzenecarboximidamide (D23)

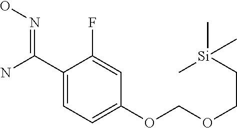

To the suspension of 2-fluoro-4-[({[2-(trimethylsilyl)ethyl]oxy}methyl)oxy]benzonitrile (D22) (3.90 g) and NaHCO₃, (8.58 g) in EtOH (200 mL) was added hydroxylamine hydrochloride (5.07 g). The mixture was refluxed for two days. The solvent was removed in vacuo and the residue was dissolved in ethyl acetate (200 mL), washed with water (2×20 mL), dried over sodium sulphate, concentrated to afford 2-fluoro-N-hydroxy-4-[({[2-(trimethylsilyl)ethyl]oxy}methyl)oxy]benzenecarboximidamide (D23) (4.38 g) as a crude product. MS (ES): C₁₃H₂₁FN₂O₃Si requires 300; found 301.2 (M+H⁺).

### Description for D24

### 5-(3-{2-Fluoro-4-[({[2-(trimethylsilyl)ethyl]oxy}methyl)oxy]phenyl}-1,2,4-oxadiazol-5-yl)-2-[(1-methylethyl)oxy]benzonitrile (D24)

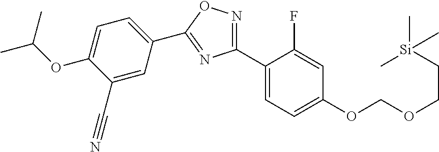

To the solution of 3-cyano-4-[(1-methylethyl)oxy]benzoic acid (2.99 g) in THF (200 mL) was added EDC (3.35 g), HOBt (2.68 g) and the resulting solution was stirred at RT for 2 hours. To the reaction solution was added 2-fluoro-N-hydroxy-4-[({[2-(trimethylsilyl)ethyl]oxy}methyl)oxy]benzenecarboximidamide (D23) (4.38 g) and the suspension was stirred at RT for another 1 hour. THF was removed in vacuo and the residue was dissolved in ethyl acetate (200 mL), washed with water (2×20 mL), dried over sodium sulphate, concentrated. The residue in dioxane (100 mL) was heated to reflux for overnight. Dioxane was removed in vacuo and the residue was purified by column to afford 5-(3-{2-fluoro-4-[({[2-(trimethylsilyl)ethyl]oxy}methyl)oxy]phenyl}-1,2,4-oxadiazol-5-yl)-2-[(1-methylethyl)oxy]benzonitrile (D24) (5.13 g). δH (CDCl₃, 400 MHz): 0.01 (9H, s), 0.97 (2H, t), 1.47 (6H, d), 3.77 (2H, t), 4.80 (1H, m), 5.28 (2H, s), 6.96 (2H, m), 7.11 (1H, d), 8.06 (1H, m), 8.33 (1H, dd), 8.43 (1H, d). MS (ES): C₂₄H₂₈FN₃O₄Si requires 469; found 470.2 (M+H⁺).

### Description for D25

### 5-[3-(2-Fluoro-4-hydroxyphenyl)-1,2,4-oxadiazol-5-yl]-2-[(1-methylethyl)oxy]benzonitrile (D25)

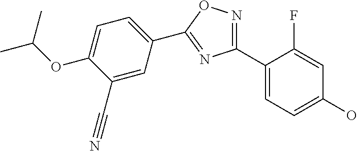

To the solution of 5-(3-{2-fluoro-4-[({[2-(trimethylsilyl)ethyl]oxy}methyl)oxy]phenyl}-1,2,4-oxadiazol-5-yl)-2-[(1-methylethyl)oxy]benzonitrile (D24) (5.13 g) in HMPA (8 mL) was added TBAF (8.57 g) at room temp. The resulting solution was heated to 40° C. for two days. The reaction solution was poured into water (100 mL), extracted with ethyl acetate (3×40 mL) and the combined organic phases was washed with water (30 mL), dried over sodium sulphate, concentrated and purified by column to afford 5-[3-(2-fluoro-4-hydroxyphenyl)-1,2,4-oxadiazol-5-yl]-2-[(1-methylethyl)oxy]benzonitrile (D25) (1.8 g) as a solid. δH (DMSO-d₆, 400 MHz): 1.41 (6H, d), 5.00 (1H, m), 6.84 (2H, m), 7.57 (1H, d), 7.97 (1H, m), 8.40 (1H, dd), 8.50 (1H, d), 10.71 (1H, s). MS (ES): C₁₈H₁₄FN₃O₃ requires 339; found 340.2 (M+H⁺).

### Description for D26

Ethyl 5-{[4-(5-{3-cyano-4-[(1-methylethyl)oxy]phenyl}-1,2,4-oxadiazol-3-yl)-3-fluorophenyl]oxy}pentanoate (D26)

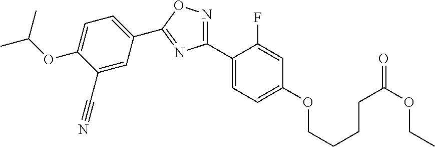

The mixture of 5-[3-(2-fluoro-4-hydroxyphenyl)-1,2,4-oxadiazol-5-yl]-2-[(1-methylethyl)oxy]benzonitrile (D25) (120 mg), ethyl 5-bromopentanoate (222 mg) and potassium carbonate (244 mg) in DMF (3 mL) was heated to 60° C. for overnight. The reaction suspension was poured into water (5 mL), extracted with ethyl acetate (2×10 mL) and the combined organic phases were dried over sodium sulphate, concentrated and purified by column to afford ethyl 5-{[4-(5-{3-cyano-4-[(1-methylethyl)oxy]phenyl}-1,2,4-oxadiazol-3-yl)-3-fluorophenyl]oxy}pentanoate (D26) (100 mg) as a pale white solid. MS (ES): C₂₅H₂₆FN₃O₅ requires 467; found 468.2 (M+H⁺).

### Description for D27

### 4-Bromo-3-(methyloxy)phenyl 4-methylbenzenesulfonate (D27)

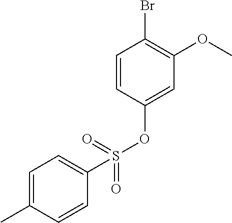

To the suspension of 4-bromo-1,3-benzenediol (5 g) and potassium carbonate (23.76 g) in acetone (150 mL) was added TsCl (5.55 g) at room temp. The resulting suspension was heated to reflux for overnight. MeI (3.31 mL) was added, and the reaction mixture was refluxed overnight again. The inorganic precipitate was filtered off, and the filtrate was concentrated under reduced pressure. The residue was diluted with water (100 mL) and extracted with ethyl acetate (3×70 mL). The combined organic solution was washed with water and dried over sodium sulphate, concentrated and purified by column to afford 4-bromo-3-(methyloxy)phenyl 4-methylbenzenesulfonate (D27) (5.3 g) as a clear oil. δH (CDCl₃, 400 MHz): 2.44 (3H, s), 3.77 (3H, s), 6.38 (1H, dd), 6.58 (1H, d), 7.31 (2H, d), 7.38 (1H, d), 7.70 (2H, d). MS (ES): C₁₄H₁₃BrO₄S requires 356; found 357.0 (M+H⁺).

### Description for D28

### 4-Bromo-3-(methyloxy)phenol (D28)

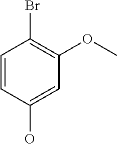

To the solution of 4-bromo-3-(methyloxy)phenyl 4-methylbenzenesulfonate (D27) (5.3 g) in methanol (100 mL) was added NaOH (30 mL, 2 M aq). The resulting solution was heated to reflux for 1.0 hour. Methanol was removed in vacuo and the residue was acidified to pH=4-5 with 2 M HCl aq, exttacted with DCM (3×50 mL). The combined organic phases were dried over sodium sulphate, concentrated to afford 4-bromo-3-(methyloxy)phenol (D28) (2.93 g). δH (CDCl₃, 400 MHz): 3.86 (3H, s), 6.32 (1H, dd), 6.45 (1H, d), 7.34 (1H, d). MS (ES): C₇H₇BrO₂ requires 202; found 203.0 (M+H⁺).

### Description for D29

### Ethyl 5-{[4-bromo-3-(methyloxy)phenyl]oxy}pentanoate (D29)

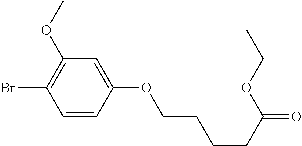

To the solution of 4-bromo-3-(methyloxy)phenol (D28) (300 mg) in N,N-dimethylformamide (2 mL) was added ethyl 5-bromopentanoate (0.710 mL) and potassium carbonate (1021 mg). The resulting suspension was heated to 45° C. for overnight. The reaction suspension was poured into water (5 mL), extracted with ethyl acetate (2×10 mL) and the combined organic phases were dried over sodium sulphate, concentrated and purified by column to afford ethyl 5-{[4-bromo-3-(methyloxy)phenyl]oxy}pentanoate (D29) (370 mg) as a clear oil. MS (ES): C₁₄H₁₉BrO₄ requires 330; found 331.1 (M4H⁺).

### Description for D30

### Ethyl 5-{[4-cyano-3-(methyloxy)phenyl]oxy}pentanoate (D30)

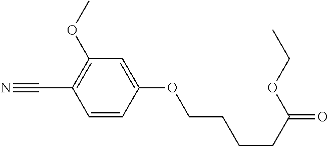

To the solution of ethyl 5-{[4-bromo-3-(methyloxy)phenyl]oxy}pentanoate (D29) (0.37 g) in N,N-dimethylacetamide (3 mL) was added dppf (0.025 g) and Pd₂ dba₃ (0.020 g), dicyanozinc (0.079 g). The reaction solution was treated by microwave (170° C., 0.5 hours). The reaction solution was poured into ethyl acetate (100 mL), washed with water, brine, dried over sodium sulphate, concentrated and purified by column to afford ethyl 5-{[4-cyano-3-(methyloxy)phenyl]oxy}pentanoate (D30) (0.2 g) as a brown oil. δH (CDCl₃, 400 MHz): 1.25 (3H, t), 1.83 (4H, m), 2.38 (2H, m), 3.89 (3H, s), 4.01 (2H, m), 4.13 (2H, q), 6.44 (1H, d), 6.48 (1H, dd), 7.45 (1H, d). MS (ES): C₁₅H₁₉NO₄ requires 277; found 278.2 (M+H⁺).

### Description for D31

### Ethyl 5-{[4-[(Z)-(hydroxyamino)(imino)methyl]-3-(methyloxy)phenyl]oxy}pentanoate] (D31)

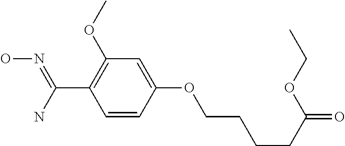

To the suspension of ethyl 5-{[4-cyano-3-(methyloxy)phenyl]oxy}pentanoate (D30) (0.2 g) and NaHCO₃, (788 mg) in EtOH (50 mL) was added hydroxylamine hydrochloride (501 mg). The mixture was refluxed for two days. The solvent was removed in vacuo and the residue was dissolved in ethyl acetate (150 mL), washed with water (2×15 mL), dried over sodium sulphate, concentrated to afford ethyl 5-{[4-[(Z)-(hydroxyamino)(imino)methyl]-3-(methyloxy)phenyl]oxy}pentanoate] (D31) (170 mg) as a crude product. MS (ES): C₁₅H₂₂N₂O₅ requires 310; found 311.2 (M+H⁺).

### Description for D32

### Ethyl 5-{[4-(5-{3-cyano-4-[(1-methylethyl)oxy]phenyl}-1,2,4-oxadiazol-3-yl)-3-(methyloxy)phenyl]oxy}pentanoate (D32)

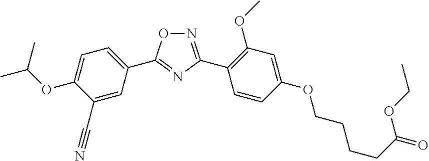

To the solution of 3-cyano-4-[(1-methylethyl)oxy]benzoic acid (112 mg) in THF (20 mL) was added EDC (126 mg), HOBt (101 mg) and the resulting solution was stirred at RT for 2 hours. To the reaction solution was added ethyl 5-{[4-[(Z)-(hydroxyamino)(imino)methyl]-3-(methyloxy)phenyl]oxy}pentanoate] (D31) (170 mg) and the suspension was stirred at RT for another 2 hours. THF was removed in vacuo and the residue was dissolved in ethyl acetate (150 mL), washed with water (2×15 mL), dried over sodium sulphate, concentrated. The residue in dioxane (50 mL) was heated to reflux for 5 hours. Dioxane was removed in vacuo and the residue was purified by column to afford ethyl 5-{[4-(5-{3-cyano-4-[(1-methylethyl)oxy]phenyl}-1,2,4-oxadiazol-3-yl)-3-(methyloxy)phenyl]oxy}pentanoate (D32) (200 mg). MS (ES): C₂₆H₂₉N₃O₆ requires 479; found 480.3 (M+H⁺).

### Description for D33

### 4-Hydroxy-2-methylbenzonitrile (D33)

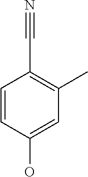

To a solution of 2-methyl-4-(methyloxy)benzonitrile (7 g) in anhydrous DCM (100 mL) was added dropwise BBr₃ (51.5 g) at −78° C. The resulting mixture was allowed to warm to RT and stirred for 24 hours. LCMS indicated the reaction was completed. Water was added dropwise slowly to quech the reaction. The mixture was extracted with EA (3×100 mL) and the combined organic layer was washed with brine, dried over sodium sulfate, and concentrated to give 5.4 g of 4-hydroxy-2-methylbenzo nitrile (D33) as a white solid. δH (DMSO-d₆, 400 MHz): 2.45 (3H, s), 6.67 (1H, d), 6.69 (1H, d), 7.51 (1H, d), 10.43 (1H, s). MS (ES): C₅H₇NO requires 133; found 134.1 (M+H^{f}).

### Description for D34

### 5-(5-amino-1,3,4-thiadiazol-2-yl)-2-[(1-methylethyl)oxy]benzonitrile (D34)

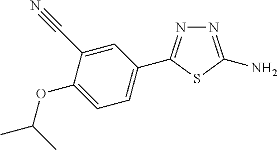

To a mixture of 3-cyano-4-[(1-methylethyl)oxy]benzoic acid (2 g) and hydrazinecarbothioamide (1.332 g) was added POCl₃ (20 mL). The reaction mixture was stirred at 90° C. for 3 h. After cooling the reaction, the mixture was concentrated to remove POCl₃. The residue was partitioned between ethyl acetate (100 mL) and water (100 mL). The organic phase was washed with water (50 mL), 2M sodium hydroxide solution (50 mL) and saturated brine (50 mL), dried over sodium sulphate and evaporated to give the crude product 5-(5-amino-1,3,4-thiadiazol-2-yl)-2-[(1-methylethyl)oxy]benzonitrile (D34) (2 g). MS (ES): C₁₂H₁₂N₄OS requires 260; found 261.1 (M+H⁺).

### Description for D35

### 5-(5-bromo-1,3,4-thiadiazol-2-yl)-2-[(1-methylethyl)oxy]benzonitrile (D35)

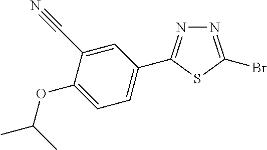

To a suspension of 5-(5-amino-1,3,4-thiadiazol-2-yl)-2-[(1-methylethyl)oxy]benzonitrile (D34) (800 mg), copper(II) bromide (1373 mg) in acetonitrile (10 mL) was added 1,1-dimethylethyl nitrite (0.737 mL). The reaction mixture was stirred at 20° C. for 1.5 h. The reaction was quenched with aqueous HCl(2M), the mixture was partitioned between ethyl acetate (50 mL) and water (25 mL). The organic phase was washed with water (25 mL) and saturated brine (25 mL), dried over sodium sulphate and evaporated in vacuo, the residue was purified by column chromatography to give 5-(5-bromo-1,3,4-thiadiazol-2-yl)-2-[(1-methylethyl)oxy]benzonitrile (D35) (800 mg). MS (ES): C₁₂H₁₀BrN₃OS requires 323; found 324.0 (M+H⁺).

### Description for D36

### 1-[(3-bromopropyl)oxy]-3-ethylbenzene (D36)

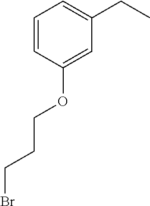

To a solution of 3-ethylphenol (5 g) in N,N-dimethylformamide (DMF) (60 mL) was added 1,3-dibromopropane (41.5 mL) and potassium carbonate (11.31 g). The resulting mixture was heated to 60° C. overnight. DMF was evaporated in vacuo and the residue was diluted with ethyl acetate (100 mL), washed with water (2*20 mL), dried over sodium sulphate, concentrated and purified by column chromatography to afford 1-[(3-bromopropyl)oxy]-3-ethylbenzene (D36) (6.88 g) as an oil. δH (CDCl₃, 400 MHz): 1.17 (3H, m), 2.24 (2H, m), 2.55 (2H, q), 3.53 (2H, t), 4.02 (2H, t), 6.69 (3H, m), 7.12 (1H, t).

### Description for D37

### 4-bromo-3-ethylphenyl 3-bromopropyl ether (D37)

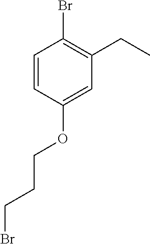

To a solution of 3-bromopropyl 3-ethylphenyl ether (D36) (6.88 g) in acetonitrile (50 mL) was added NBS (5.29 g) portionwise under ice water cooling. After addition, the reaction solution was stirred at room temperature for 2 h. Water (20 mL) was added and the resulting solution was evaporated in vacuo, the residue was diluted with ethyl acetate (100 mL). The organic phase was washed with brine, dried over sodium sulphae, concentrated and purified by column chromatography to afford 4-bromo-3-ethylphenyl 3-bromopropyl ether (D37) (5.3 g) as an oil. δH (CDCl₃, 400 MHz): 1.23 (3H, t), 2.32 (2H, m), 2.72 (2H, q), 3.61 (2H, t), 4.08 (2H, t), 6.63 (1H, dd), 6.81 (1H, d), 7.41 (1H, d).

### Description for D38

### 3-[(4-bromo-3-ethylphenyl)oxy]-N-methyl-1-propanamine (D38)

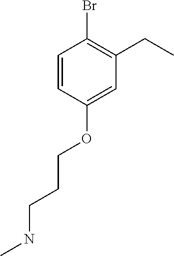

To a solution of 4-bromo-3-ethylphenyl 3-bromopropyl ether (D37) (2 g) in tetrahydrofuran (THF) (10 mL) was added potassium carbonate (6.01 g) and methylamine hydrochloride (2.097 g). The reaction mixture was stirred at 60° C. in a sealed tube for overnight. THF was removed in vacuo and the residue was dissolved in ethyl acetate (100 mL), washed with water (2*10 mL), dried over sodium sulphate, concentrated to afford 3-[(4-bromo-3-ethylphenyl)oxy]-N-methyl-1-propanamine (D38) (1.8 g). MS (ES): C₁₂H₁₈BrNO requires 271; found 272.1 (M+H⁺).

### Description for D39

### 1,1-dimethylethyl{3-[(4-bromo-3-ethylphenyl)oxy]propyl}methylcarbamate (D39)

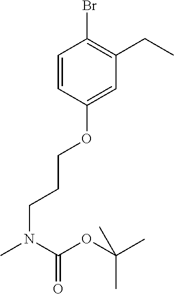

To a solution of 3-[(4-bromo-3-ethylphenyl)oxy]-N-methyl-1-propanamine (1.8 g) in tetrahydrofuran (THF) (20 mL) was added Et₃N (4.61 mL), DMAP (0.040 g), and Boc₂O (1.843 mL). The reaction was stirred at room temp for 4 h. Water (30 mL) was added and the mixture was extracted with ethyl acetate (100 mL). The organic phase was dried over sodium sulphate, concentrated and purified by column chromatography to afford 1,1-dimethylethyl {3-[(4-bromo-3-ethylphenyl)oxy]propyl}methylcarbamate (D39) (1.6 g) as an oil. MS (ES): C₁₇H₂₆BrNO₃ requires 371; found 394.1 (M+Na⁺).

### Description for D40

### 1,1-dimethylethyl(3-{[3-ethyl-4-(4,4,5,5-tetramethyl-1,3,2-dioxaborolan-2-yl)phenyl]oxy}propyl)methylcarbamate (D40)

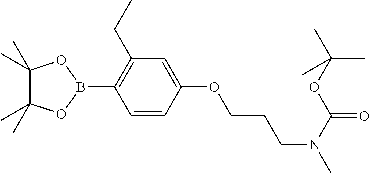

To a solution of 1,1-dimethylethyl {3-[(4-bromo-3-ethylphenyl)oxy]propyl}methylcarbamate (D39) (1.6 g) in N,N-dimethylformamide (DMF) (20 mL) was added 4,4,4′,4′,5,5,5′,5′-octamethyl-2,2′-bi-1,3,2-dioxaborolane (1.637 g), potassium acetate (1.476 g) and PdCl₂(dppf)-CH₂Cl₂ adduct (0.351 g). The reaction solution was degassed and heated to 80° C. overnight under nitrogen. After cooling the reaction, DMF was removed in vacuo and the residue was dissolved in ethyl acetate (100 mL), filtered and washed with water (2*15 mL), dried over sodium sulphate, concentrated and purified by column chromatography to afford 1,1-dimethylethyl (3-{[3-ethyl-4-(4,4,5,5-tetramethyl-1,3,2-dioxaborolan-2-yl)phenyl]oxy}propyl)methylcarbamate (D40) (1.9 g) as an oil. δH (CDCl₃, 600 MHz): 1.20 (3H, t), 1.35 (12H, s), 1.45 (9H, s), 2.02 (2H, s br), 2.91 (5H, m), 3.43 (2H, t), 4.01 (2H, s), 6.72 (2H, m), 7.74 (1H, d).

### Description for D41

### 1,1-dimethylethyl (3-{[4-(5-{3-cyano-4-[(1-methylethyl)oxy]phenyl}-1,3,4-thiadiazol-2-yl)-3-ethylphenyl]oxy}propyl)methylcarbamate (D41)

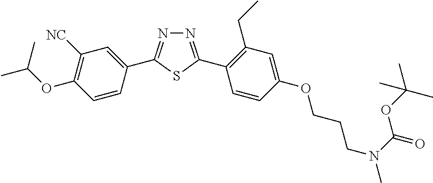

To a solution of 1,1-dimethylethyl (3-{[3-ethyl-4-(4,4,5,5-tetramethyl-1,3,2-dioxaborolan-2-yl)phenyl]oxy}propyl)methylcarbamate (D40) (100 mg) in N,N-dimethylformamide (DMF) (3 mL) and water (0.750 mL) was added 5-(5-bromo-1,3,4-thiadiazol-2-yl)-2-[(1-methylethyl)oxy]benzonitrile (D35) (77 mg), tripotassium phosphate (127 mg) and Pd(Ph₃P)₄ (27.6 mg) under nitrogen. The reaction vessel was sealed and heated under microwave at 120° C. for 15 min. After cooling the reaction, the mixture was diluted with ethyl acetate (20 mL), washed with water (2*5 mL), dried over sodium sulphate, concentrated and purified by column chromatography to afford 1,1-dimethylethyl (3-{[4-(5-{3-cyano-4-[(1-methylethyl)oxy]phenyl}-1,3,4-thiadiazol-2-yl)-3-ethylphenyl]oxy}propypmethylcarbamate (D41) (128 mg) as an oil. MS (ES): C₂₉H₃₆N₄O₄S requires 536; found 537.3 (M+H⁺).

### Description for D42

### 5-(5-amino-1,3,4-oxadiazol-2-yl)-2-[(1-methylethyl)oxy]benzonitrile (D42)

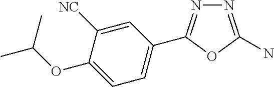

To a suspension of 3-cyano-4-[(1-methylethyl)oxy]benzoic acid (2 g) in phosphorus oxychloride (10 mL) was added hydrazinecarboxamide hydrochloride (1.630 g). The reaction mixture was heated at 90° C. for 3 h. The reaction mixture was cooled to room temperature, the solvent was removed in vacuo. The residue was poured into ice carefully and neutralized with 2 M NaOH to pH=7. The aqueous layer was extracted with EtOAc, the organic layer was dried over sodium sulfate and evaporated in vacuo to afford the crude product 5-(5-amino-1,3,4-oxadiazol-2-yl)-2-[(1-methylethyl)oxy]benzonitrile (D42) (1.93 g). The crude product was used directly for the next step.

### Description for D43

### 5-(5-bromo-1,3,4-oxadiazol-2-yl)-2-[(1-methylethyl)oxy]benzonitrile (D43)

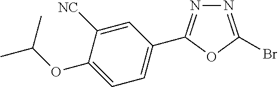

To a mixture of 5-(5-amino-1,3,4-oxadiazol-2-yl)-2-[(1-methylethyl)oxy]benzonitrile (D42) (1.93 g) and copper(II) bromide (3.53 g) in acetonitrile (100 mL), 1,1-dimethylethyl nitrite (1.895 mL) was added in one portion at RT and the reaction mixture was stirred overnight. The reaction mixture was diluted with EtOAc (20 mL) and 1M HCl (10 mL) was added. The organic layer was washed with water and saturated NaHCO₃, then dried over sodium sulphate. The solvent was removed in vacuo. The crude product was purified by column chromatography to give 5-(5-bromo-1,3,4-oxadiazol-2-yl)-2-[(1-methylethyl)oxy]benzonitrile (D43) (1.1 g). MS (ES): C₁₂H₁₀BrN₃O₂ requires 307; found 308.0 (M+H⁺).

### Description for D44

### 1,1-dimethylethyl (3-{[4-(5-{3-cyano-4-[(1-methylethyl)oxy]phenyl}-1,3,4-oxadiazol-2-yl)-3-ethylphenyl]oxy}propyl)methylcarbamate (D44)

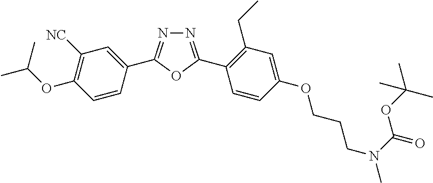

To a solution of 1,1-dimethylethyl (3-{[3-ethyl-4-(4,4,5,5-tetramethyl-1,3,2-dioxaborolan-2-yl)phenyl]oxy}propyl)methylcarbamate (D40) (100 mg) in N,N-dimethylformamide (DMF) (3 mL) and water (0.750 mL) was added 5-(5-bromo-1,3,4-oxadiazol-2-yl)-2-[(1-methylethyl)oxy]benzonitrile (D43) (73.5 mg), tripotassium phosphate (127 mg) and Pd(Ph₃P)₄ (27.6 mg) under nitrogen. The reaction vessel was sealed and heated under microwave at 120° C. for 15 min. After cooling the reaction, the mixture was diluted with ethyl acetate (20 mL), washed with water (2*5 mL), dried over sodium sulphate, concentrated and purified by column chromatography to afford 1,1-dimethylethyl (3-{[4-(5-{3-cyano-4-[(1-methylethyl)oxy]phenyl}-1,3,4-oxadiazol-2-yl)-3-ethylphenyl]oxy}propyl)methylcarbamate (D44) (80 mg) as an oil. MS (ES): C₂₉H₃₆N₄O₅ requires 520; found 543.3 (M+Na⁺).

### Description for D45

### ethyl 4-[(3-ethylphenyl)oxy]butanoate (D45)

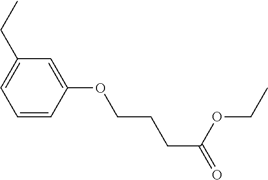

To a solution of 3-ethylphenol (5 g) in N,N-Dimethylformamide (DMF) (70 mL) was added potassium carbonate (7.35 g) and ethyl 4-bromobutanoate (8.38 g). The reaction solution was stirred at 80° C. overnight. The reaction solution was diluted with ethyl acetate (200 mL), washed with water (3*30 mL), dried over sodium sulphate and concentrated to afford ethyl 4-[(3-ethylphenyl)oxy]butanoate (D45) (9.67 g) as a crude product. MS (ES): C₁₄H₂₀O₃ requires 236; found 237.2 (M+Na⁺).

### Description for D46

### ethyl 4-[(4-bromo-3-ethylphenyl)oxy]butanoate (D46)

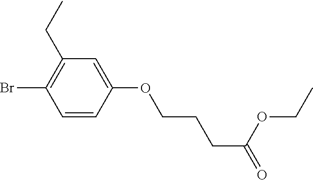

To a solution of ethyl 4-[(3-ethylphenyl)oxy]butanoate (D45) (9.7 g) in acetonitrile (60 mL) was added NBS (7.7 g) under ice-water cooling. After addition, the reaction solution was warmed to room temperature and stirred for 1 h. The reaction solution was poured into water (50 mL), extracted with ethyl acetate (2*30 mL) and the combined organic phases were dried over sodium sulphate, concentrated to afford ethyl 4-[(4-bromo-3-ethylphenyl)oxy]butanoate (D46) (7.8 g) as a crude product. SH (CDCl₃, 400 MHz): 1.25 (6H, m), 2.10 (2H, m), 2.51 (2H, t), 2.71 (2H, q), 3.98 (2H, t), 4.15 (2H, q), 6.61 (1H, dd), 6.78 (1H, d), 7.39 (1H, d).

### Description for D47

### ethyl 4-{[-ethyl-4-(4,4,5,5-tetramethyl-1,3,2-dioxaborolan-2-yl)phenyl]oxy}butanoate (D47)

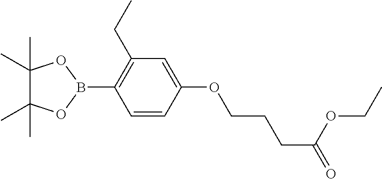

To a solution of ethyl 4-[(4-bromo-3-ethylphenyl)oxy]butanoate (D46) (7.8 g) in N,N-dimethylformamide (DMF) (70 mL) was added 4,4,4′,4′,5,5,5′,5′-octamethyl-2,2′-bi-1,3,2-dioxaborolane (9.43 g), potassium acetate (8.50 g) and PdCl₂(dppf)-CH₂Cl₂ adduct (2.021 g). The resulting mixture was degassed and heated to 80° C. under nitrogen for overnight. After cooling the reaction, DMF was removed in vacuo and the residue was dissoved in ethyl acetate (100 mL), washed with water (2*20 mL), dried over sodium sulphate, concentrated and purified by column chromatography to afford ethyl 4-{[3-ethyl-4-(4,4,5,5-tetrannethyl-1,3,2-dioxaborolan-2-yl)phenyl]oxy}butanoate (D47) (7 g) as a colorless oil. δH (CDCl₃, 400 MHz): 1.18 (3H, m), 1.26 (3H, m), 1.33 (12H, s), 2.11 (2H, m), 2.52 (2H, t), 2.89 (2H, q), 4.03 (2H, t), 4.15 (2H, q), 6.71 (2H, m), 7.72 (1H, d).

### Description for D48

### ethyl 4-{[4-(5-{3-cyano-4-[(1-methylethyl)oxy]phenyl}-1,3,4-oxadiazol-2-yl)-3-ethylphenyl]oxy}butanoate (D48)

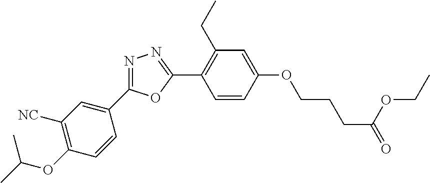

To a solution of ethyl 4-{[3-ethyl-4-(4,4,5,5-tetramethyl-1,3,2-dioxaborolan-2-yl)phenyl]oxy}butanoate (D47) (100 mg) in N,N-dimethylformamide (DMF) (3 mL) and water (0.750 mL) was added 5-(5-bromo-1,3,4-oxadiazol-2-yl)-2-[(1-methylethyl)oxy]benzonitrile (D43) (85 mg), tripotassium phosphate (146 mg) and Pd(Ph₃P)₄ (31.9 mg) under nitrogen. The reaction vessel was sealed and heated under microwave at 120° C. for 15 min. After cooling the reaction, the mixture was diluted with ethyl acetate (20 mL), washed with water (2*5 mL), dried over sodium sulphate, concentrated and purified by column chromatography to afford ethyl 4-{[4-(5-{3-cyano-4-[(1-methylethyl)oxy]phenyl}-1,3,4-oxadiazol-2-yl)-3-ethylphenyl]oxy}butanoate (D48)

(128 mg). MS (ES): C₂₆H₂₉H₃O₅ requires 463; found 464.3 (M+H⁺).

### Description for D49

### ethyl 4-{[4-(5-{3-cyano-4-[(1-methylethyl)oxy]phenyl}-1,3,4-thiadiazol-2-yl)-3-ethylphenyl]oxy}butanoate (D49)

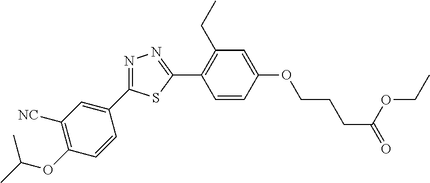

To a solution of ethyl 4-{[3-ethyl-4-(4,4,5,5-tetramethyl-1,3,2-dioxaborolan-2-yl)phenyl]oxy}butanoate (D47) (100 mg) in 1,2-dimethoxyethane (DME) (3 mL) and water (0.750 mL) was added 5-(5-bromo-1,3,4-thiadiazol-2-yl)-2-[(1-methylethyl)oxy]benzonitrile (D35) (89 mg), Cs₂CO₃ (270 mg) and PdCl₂(dppf)-CH₂Cl₂ adduct (22.54 mg) under nitrogen. The reaction vessel was sealed and heated under microwave at 120° C. for 30 min. After cooling the reaction, the mixture was diluted with ethyl acetate (20 mL), washed with water (2*5 mL), dried over sodium sulphate, concentrated and purified by column chromatography to afford ethyl 4-{[4-(5-{3-cyano-4-[(1-methylethyl)oxy]phenyl}-1,3,4-thiadiazol-2-yl)-3-ethylphenyl]oxy}butanoate (D49) (50 mg). MS (ES): C₂₆H₂₃N₃O₄S requires 479; found 480.2 (M+H⁺).

### Description for D50

### 5-(3-{4-[(4-bromobutyl)oxy]-2-chlorophenyl}-1,2,4-oxadiazol-5-yl)-2-[(1-methylethyl)oxy]benzonitrile (D50)

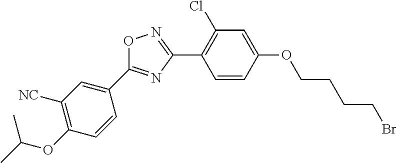

To a solution of 5-[3-(2-chloro-4-hydroxyphenyl)-1,2,4-oxadiazol-5-yl]-2-[(1-methylethyl)oxy]benzonitrile (D13) (500 mg) in N,N-dimethylformamide (DMF) (2 mL) was added potassium carbonate (971 mg) and 1,4-dibromobutane (3.33 mL). The resulting solution was stirred at 60° C. overnight. After cooling the reaction, ethyl acetate (20 mL) was added to the reaction solution, washed with water (2*7 mL), dried over sodium sulphate, concentrated to afford 5-(3-{4-[(4-bromobutyl)oxy]-2-chlorophenyl}-1,2,4-oxadiazol-5-yl)-2-[(1-methylethyl)oxy]benzonitrile (D50) (690 mg) as a crude product. MS (ES): C₂₂H₂₁BrClN₃O₃ requires 489; found 490.1 (M+H⁺).

### Description for D51

### ethyl 4-{[4-(5-{3-cyano-4-[(1-methylethyl)oxy]phenyl}-1,2,4-oxadiazol-3-yl)-3-fluorophenyl]oxy}butanoate (D51)

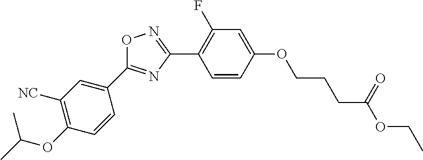

To a solution of 5-[3-(2-fluoro-4-hydroxyphenyl)-1,2,4-oxadiazol-5-yl]-2-[(1-methylethyl)oxy]benzonitrile (D25) (100 mg) in N,N-dimethylformamide (DMF) (2 mL) was added potassium carbonate (61.1 mg) and ethyl 4-bromobutanoate (63.2 mg). The resulting solution was heated to 60° C. overnight. The reaction solution was diluted with ethyl acetate (20 mL), washed with water (2*8 mL), dried over sodium sulphate, concentrated to afford ethyl 4-{[4-(5-{3-cyano-4-[(1-methylethyl)oxy]phenyl}-1,2,4-oxadiazol-3-yl)-3-fluorophenyl]oxy}butanoate (D51) (120 mg) as a crude product. MS (ES): C₂₄H₂₄FN₃O₅ requires 453; found 454.2 (M+H⁺).

### Description for D52

### ethyl {[4-(5-{3-cyano-4-[(1-methylethyl)oxy]phenyl}-1,2,4-oxadiazol-3-yl)-3-fluorophenyl]oxy}acetate (D52)

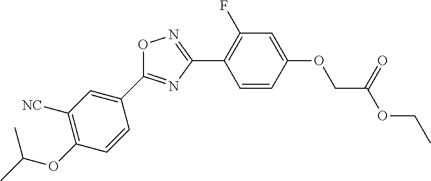

To a solution of 5-[3-(2-fluoro-4-hydroxyphenyl)-1,2,4-oxadiazol-5-yl]-2-[(1-methylethyl)oxy]benzonitrile (D25) (100 mg) in N,N-dimethylformamide (DMF) (2 mL) was added potassium carbonate (61.1 mg) and ethyl bromoacetate (54.1 mg). The resulting solution was heated to 60° C. overnight. The reaction solution was diluted with ethyl acetate (20 mL), washed with water (2*8 mL), dried over sodium sulphate, concentrated to afford ethyl {[4-(5-{3-cyano-4-[(1-methylethyl)oxy]phenyl}-1,2,4-oxadiazol-3-yl)-3-fluorophenyl]oxy}acetate (D52) (120 mg) as a curde product. MS (ES): C₂₂H₂₀FN₃O₅ requires 425; found 426.2 (M+H⁺).

### Description for D53

### ethyl 4-{[4-(5-{3-cyano-4-[(1-methylethyl)oxy]phenyl}-1,2,4-oxadiazol-3-yl)phenyl]oxy}butanoate (D53)

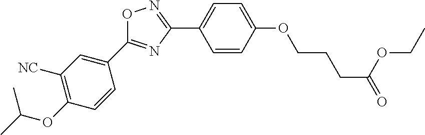

To a solution of 5-[3-(4-hydroxyphenyl)-1,2,4-oxadiazol-5-yl]-2-[(1-methylethyl)oxy]benzonitrile (D18) (80 mg) in N,N-dimethylformamide (DMF) (2 mL) was added potassium carbonate (51.6 mg) and ethyl 4-bromobutanoate (53.4 mg). The reaction solution was heated at 60° C. for 2 d. To the reaction solution was added ethyl acetate (30 mL), washed with water (2*8 mL), dried over sodium sulphate, concentrated to afford ethyl 4-{[4-(5-{3-cyano-4-[(1-methylethyl)oxy]phenyl}-1,2,4-oxadiazol-3-yl)phenyl]oxy}butanoate (D53) (108 mg) as a brown oil. MS (ES): C₂₄H₂₆N₃O₅ requires 435; found 436.2 (M+H⁺).

### Description for D54

### ethyl {[4-(5-{3-cyano-4-[(1-methylethyl)oxy]phenyl}-1,2,4-oxadiazol-3-yl)phenyl]oxy}acetate (D54)

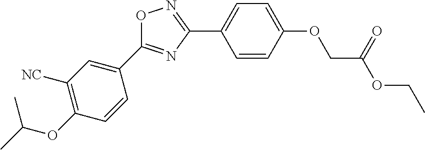

To a solution of 5-[3-(4-hydroxyphenyl)-1,2,4-oxadiazol-5-yl]-2-[(1-methylethyl)oxy]benzonitrile (D18) (80 mg) in N,N-dimethylformamide (DMF) (2 mL) was added potassium carbonate (51.6 mg) and ethyl bromoacetate (45.7 mg). The resulting solution was stirred at 60° C. for 2 d. To the reaction solution was added ethyl acetate (30 mL), washed with water (2*5 mL), dried over sodium sulphate, concentrated to afford ethyl {[4-(5-{3-cyano-4-[(1-methylethyl)oxy]phenyl}-1,2,4-oxadiazol-3-yl)phenyl]oxy}acetate (D54) (80 mg) as a white solid. MS (ES): C₂₂H₂₁N₃O₅ requires 407; found 408.1 (M+H⁺).

### Description for D55

### 1-ethyl-3-[(phenylmethyl)oxy]benzene (D55)

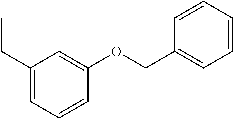

To the solution of 3-ethylphenol (13.23 mL) in N,N-dimethylformamide (DMF) (150 mL) was added potassium carbonate (23.08 g) and benzyl chloride (13.06 mL). The reaction solution was heated to 50° C. for 2 d. After cooling the reaction, the mixture was quenched with water (200 mL) and extracted with ethyl acetate (3*150 mL). The combined organic phases were washed with water (3*50 mL), dried over sodium sulphate, concentrated to afford 1-ethyl-3-[(phenylmethyl)oxy]benzene (D55) (26.5 g) as a brown oil.

### Description for D56

### 1-bromo-2-ethyl-4-[(phenylmethyl)oxy]benzene (D56)

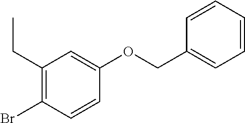

To the solution of 1-ethyl-3-[(phenylmethyl)oxy]benzene (D55) (28.4 g) in Acetonitrile (250 mL) was added NBS (24.05 g) portionwise in 1 hour under ice-water cooling, the temperature was controlled between 20-25° C. The reaction solution was stirred for 1 hour at 20° C. Acetonitrile was removed in vacuo and the residue was added water (200 mL), extracted with ethyl acetate (3*120 mL). The combined organic phases were washed with water (3*50 mL) and dried over sodium sulphate, concentrated to afford 1-bromo-2-ethyl-4-[(phenylmethyl)oxy]benzene (D56) (40.5 g) as a brown oil. δH (CDCl₃, 400 MHz): 1.25 (3H, t), 2.75 (2H, m), 5.06 (2H, s), 6.72 (1H, dd), 6.91 (1H, d), 7.35 (1H, m), 7.43 (5H, m).

### Description for D57

### 2-ethyl-4-[(phenylmethyl)oxy]benzonitrile (D57)

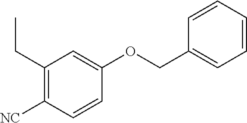

To the solution of 1-bromo-2-ethyl-4-[(phenylmethyl)oxy]benzene (D56) (40.5 g) in N,N-dimethylacetamide (DMA) (150 mL) was added DPPF (3.08 g), Pd₂(dba)₃ (2.55 g) and dicyanozinc (11.43 g). The reaction suspension was heated to 150° C. for 4 h. After cooling the reaction, the solution was filtered and the filtrate was added water (150 mL), extracted with ethyl acetate (3*120 mL). The combined organic phases were washed with water (3*50 mL), dried over sodium sulphate, concentrated and purified by column chromatography to afford 2-ethyl-4-[(phenylmethyl)oxy]benzonitrile (D57) (25.2 g) as a brown oil. MS (ES): C₁₆H₁₅NO requires 237; found 238.2 (M+H⁺).

### Description for D58

### 2-ethyl-4-hydroxybenzonitrile (D58)

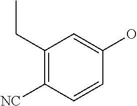

To the solution of 2-ethyl-4-[(phenylmethyl)oxy]benzonitrile (D57) (25.2 g) in methanol (200 mL) was added Pd/C (2.260 g). The reaction mixture was stirred under hydrogen balloon at room temperature for 2 d. After filtration, the filtrate was concentrated to afford 2-ethyl-4-hydroxybenzonitrile (D58) (15.4 g). MS (ES): C₉H₉NO requires 147; found 148.2 (M+H⁺).

### Description for D59

### 2-ethyl-4-[({[2-(trimethylsilyl)ethyl]oxy}methyl)oxy]benzonitrile (D59)

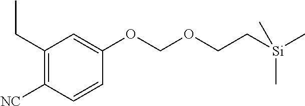

To the solution of 2-ethyl-4-hydroxybenzonitrile (D58) (15.87 g) in dichloromethane (DCM) (300 mL) was added Hunig's base (28.2 mL), followed by SEMCl (21.04 mL) dropwise under ice-water cooling. After addition, the reaction solution was warmed to room temperature. The reaction solution was washed with water, dried over sodium sulphate, concentrated to afford 2-ethyl-4-[([{2-(trimethylsilyl)ethyl]oxy}methyl)oxy]benzonitrile (D59) (29.9 g). MS (ES): C₁₅H₂₃NO₂Si requires 277; found 278.2 (M+H⁺).

### Description for D60

### 2-ethyl-N-hydroxy-4-[({[2-(trimethylsilyl)ethyl]oxy}methyl)oxy]benzenecarboximidamide (D60)

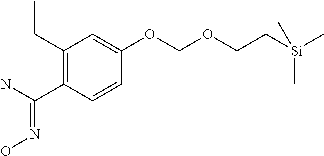

To the solution of 2-ethyl-4-[({[2-(trimethylsilyl)ethyl]oxy}methyl)oxy]benzonitrile (D59) (30 g) in ethanol (200 mL) was added sodium bicarbonate (118 g) and hydroxylamine hydrochloride (75 g). The reaction mixture was stirred at 77° C. for 2 d. After filtration, the filtrate was concentrated and the residue was dissolved in ethyl acetate (200 mL), washed with water (3*20 mL), dried over sodium sulphate, concentrated to afford 2-ethyl-N-hydroxy-4[({[2-(trimethylsilyl)-ethyl]oxy}methyl)oxy]benzenecarboximidamide (D60) (37.2 g). MS (ES): C₁₆H₂₆N₂O₃Si requires 310; found 311.3 (M+H⁺).

### Description for D61

### 5-{3-chloro-4-[(1-methylethyl)oxy]phenyl}-3-{2-ethyl-4-[({[2-(trimethylsilyl)ethyl]oxy}methyl)oxy]phenyl}-1,2,4-oxadiazole (D61)

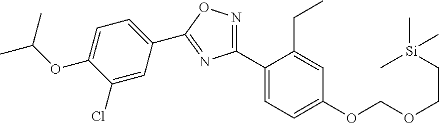

To the solution of 3-chloro-4-[(1-methylethyl)oxy]benzoic acid (7.72 g) in tetrahydrofuran (THF) (200 mL) was added HOBT (7.16 g) and EDC (8.96 g). The reaction solution was stirred at room temperature for 2 h. To the reaction solution was added 2-ethyl-N-hydroxy-4-[({[2-(trimethylsilyl)ethyl]oxy}methyl)oxy]benzenecarboximidamide (D60) (18.6 g) and the reaction solution was stirred at room temperature for another 2 h. THF was removed in vacuo and the residue was dissolved in ethyl acetate (200 mL), washed with water, dried over sodium sulphate and concentrated. The residue in dioxane (100 mL) was heated to 110° C. for 5 h. Dioxane was removed in vacuo and the residue was purified by column chromatography to afford 5-{3-chloro-4-[(1-methylethyl)oxy]phenyl}-3-{2-ethyl-4-[({[2-(trimethylsilyl)ethyl]oxy}methyl)oxy]phenyl}-1,2,4-oxadiazole (D61) (8.6 g). δH (CDCl₃, 600 MHz): 0.01 (9H, s), 0.96 (2H, t), 1.27 (3H, t), 1.43 (6H, d), 3.03 (2H, q), 3.77 (2H, t), 4.69 (1H, m), 5.27 (2H, s), 6.99 (2H, m), 7.04 (1H, d), 7.96 (1H, d), 8.03 (1H, dd), 8.21 (1H, d). MS (ES): C₂₅H₃₃ClN₂O₄Si requires 488; found 489.2 (M+H⁺).

### Description for D62

### 4-(5-{3-chloro-4-[(1-methylethyl)oxy]phenyl}-1,2,4-oxadiazol-3-yl)-3-ethylphenol (D62)

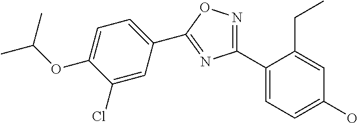

To the solution of 5-{3-chloro-4-[(1-methylethyl)oxy]phenyl}-3-{2-ethyl-4-[({[2-(trimethylsilyl)ethyl]oxy}methyl)oxy]phenyl}-1,2,4-oxadiazole (D61) (8.6 g) in HMPA (15 mL) was added TBAF (13.79 g). The reaction solution was heated to 40° C. for 2 d. The reaction solution was diluted with ethyl acetate (200 mL), washed with water (3*20 mL), dried over sodium sulphate, concentrated and purified by column chromatography to afford 4-(5-{3-chloro-4-[(1-methylethyl)oxy]phenyl}-1,2,4-oxadiazol-3-yl)-3-ethylphenol (D62) (4 g) as a brown oil. δH (CDCl₃, 600 MHz): 1.23 (3H, t), 1.41 (6H, d), 2.99 (2H, q), 4.67 (1H, m), 5.62 (1H, s br), 6.75 (1H, dd), 6.80 (1H, d), 7.01 (1H, d), 7.88 (1H, d), 8.00 (1H, dd), 8.19 (1H, d). MS (ES): C₁₉H₁₉ClN₂O₃ requires 358; found 359.1 (M+H⁺).

### Description for D63

### 3-{4-[(2-bromoethyl)oxy]-2-ethylphenyl}-5-(3-chloro-4-[(1-methylethyl)oxy]phenyl)-1,2,4-oxadiazole (D63)

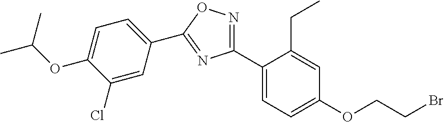

To the solution of 4-(5-{3-chloro-4-[(1-methylethyl)oxy]phenyl}-1,2,4-oxadiazol-3-yl)-3-ethylphenol (D62) (130 mg) in N,N-dimethylformamide (DMF) (2 mL) was added potassium carbonate (150 mg) and 1,2-dibromoethane (0.625 mL). The resulting solution was stirred at 60° C. overnight. The reaction solution was diluted with ethyl acetate (20 mL), washed with water (3*5 mL), dried over sodium sulphate, concentrated to afford 3-{4-[(2-bromoethyl)oxy]-2-ethylphenyl}-5-{3-chloro-4-[(1-methylethyl)oxy]phenyl}-1,2,4-oxadiazole (D63) (120 mg) as a pale-white solid. MS (ES): C₂₁H₂₂BrClN₂O₃ requires 464; found 465.1 (M+H⁺).

### Description for D64

### 5-(3-{2-ethyl-4-[({[2-(trimethylsilyl)ethyl]oxy}methyl)oxy]phenyl}-1,2,4-oxadiazol-5-yl)-2-[(1-methylethyl)oxy]benzonitrile (D64)

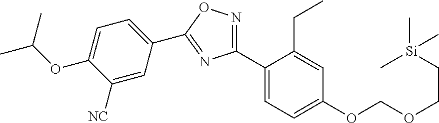

To the solution of 3-cyano-4-[(1-methylethyl)oxy]benzoic acid (8.11 g) in tetrahydrofuran (THF) (120 mL) was added HOBT (7.16 g) and EDC (8.96 g). The reaction solution was stirred at room temperature for 2 h. To the reaction solution was added 2-ethyl-N-hydroxy-4-[({[2-(trimethylsilyl)ethyl]oxy}methyl)oxy]benzenecarboximidamide (D60) (18.6 g) and the reaction solution was stirred at room temperature for another 2 h. THF was removed in vacuo and the residue was dissolved in ethyl acetate (200 mL), washed with water, dried over sodium sulphate, concentrated. The residue in dioxane (100 mL) was heated to 110° C. for 5 h. Dioxane was removed in vacuo and the residue was purified by column chromatography to afford 5-(3-{2-ethyl-4-[({[2-(trimethylsilyl)ethyl]oxy}methyl)oxy]phenyl}-1,2,4-oxadiazol-5-yl)-2-[(1-methylethyl)oxy]benzonitrile (D64) (7.3 g). δH (CDCl₃, 600 MHz): 0.01 (9H, s), 0.96 (2H, t), 1.26 (3H, t), 1.46 (6H, d), 3.03 (2H, q), 3.77 (2H, t), 4.77 (1H, m), 5.27 (2H, s), 7.00 (2H, m), 7.09 (1H, d), 7.96 (1H, d), 8.31 (1H, dd), 8.39 (1H, d). MS (ES): C₂₅H₃₃N₃O₄Si requires 479; found 480.3 (M+H⁺).

### Description for D65

### 5-[3-(2-ethyl-4-hydroxyphenyl)-1,2,4-oxadiazol-5-yl]-2-[(1-methylethyl)oxy]benzonitrile (D65)

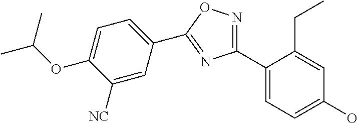

To the solution of 5-(3-{2-ethyl-4-[({[2-(trimethylsilyl)ethyl]oxy}methyl)oxy]phenyl}-1,2,4-oxadiazol-5-yl)-2-[(1-methylethyl)oxy]benzonitrile (D64) (7.3 g) in HMPA (15 mL) was added TBAF (11.94 g). The reaction solution was heated to 40° C. for 2 d. The reaction solution was diluted with ethyl acetate (200 mL), washed with water (3*20 mL), dried over sodium sulphate, concentrated and purified by column chromatography to afford 5-[3-(2-ethyl-4-hydroxyphenyl)-1,2,4-oxadiazol-5-yl]-2-[(1-methylethyl)oxy]benzonitrile (D65) (3.72 g) as a pale-white solid. δH (CDCl₃, 600 MHz): 1.25 (3H, t), 1.46 (6H, d), 3.02 (2H, q), 4.77 (1H, m), 5.10 (1H, s br), 6.78 (1H, dd), 6.82 (1H, d), 7.09 (1H, d), 7.94 (1H, d), 8.30 (1H, dd), 8.39 (1H, d). MS (ES): C₂₀H₁₉N₃O₃ requires 349; found 350.2 (M+H⁺).

### Description for D66

### ethyl {[4-(5-{3-cyano-4-[(1-methylethyl)oxy]phenyl}-1,2,4-oxadiazol-3-yl)-3-ethylphenyl]oxy}acetate (D66)

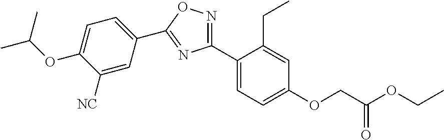

To a solution of 5-[3-(2-ethyl-4-hydroxyphenyl)-1,2,4-oxadiazol-5-yl]-2-[(1-methylethyl)oxy]benzonitrile (D65) (90 mg) in N,N-dimethylformamide (DMF) (2 mL) was added potassium carbonate (53.4 mg) and ethyl bromoacetate (47.3 mg). The reaction solution was stirred at 60° C. overnight. The reaction solution was diluted with ethyl acetate (30 mL), washed with water (2*8 mL), dried over sodium sulphate, concentrated to afford ethyl {[4-(5-{3-cyano-4-[(1-methylethyl)oxy]phenyl}-1,2,4-oxadiazol-3-yl)-3-ethylphenyl]oxy}acetate (D66) (112 mg) as a crude product. MS (ES): C₂₄H₂₅N₃O₅ requires 435; found 436.2 (M+H⁺).

### Description for D67

### ethyl {[4-(5-{3-chloro-4-[(1-methylethyl)oxy]phenyl}-1,2,4-oxadiazol-3-yl)-3-ethylphenyl]oxy}acetate (D67)

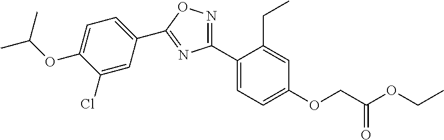

To a solution of 4-(5-{3-chloro-4-[(1-methylethyl)oxy]phenyl}-1,2,4-oxadiazol-3-yl)-3-ethylphenol (D62) (110 mg) in N,N-dimethylformamide (DMF) (2 mL) was added potassium carbonate (63.6 mg) and ethyl bromoacetate (56.3 mg). The reaction solution was heated to 60° C. overnight. The reaction solution was diluted with ethyl acetate (30 mL), washed with water (2*8 mL), dried over sodium sulphate, concentrated to afford ethyl {[4-(5-{3-chloro-4-[(1-methylethyl)oxy]phenyl}-1,2,4-oxadiazol-3-yl)-3-ethylphenyl]oxy}acetate (D67) (136 mg) as a crude product. MS (ES): C₂₃H₂₅ClN₂O₅ requires 444; found 445.2 (M+H⁺).

### Description for D68

### ethyl 5-{[4-(5-{3-chloro-4-[(1-methylethyl)oxy]phenyl}-1,2,4-oxadiazol-3-yl)-3-ethylphenyl]oxy}pentanoate (D68)

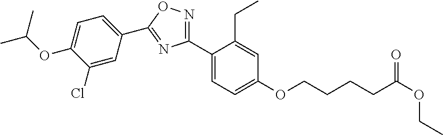

To a solution of 4-(5-{3-chloro-4-[(1-methylethyl)oxy]phenyl}-1,2,4-oxadiazol-3-yl)-3-ethylphenol (D62) (120 mg) in N,N-dimethylformamide (DMF) (2 mL) was added potassium carbonate (69.3 mg) and ethyl 5-bromopentanoate (84 mg). The reaction solution was heated to 60° C. overnight. The reaction solution was diluted with ethyl acetate (20 mL), washed with water (2*8 mL), dried over sodium sulphate, concentrated to afford ethyl 5-{[4-(5-{3-chloro-4-[(1-methylethyl)oxy]phenyl}-1,2,4-oxadiazol-3-yl)-3-ethylphenyl]oxy}pentanoate (D68) (163 mg) as a crude product. MS (ES): C₂₆H₃₁ClN₂O₅ requires 486; found 487.2 (M+H⁺).

### Description for D69

### ethyl 4-{[4-(5-{3-chloro-4-[(1-methylethyl)oxy]phenyl}-1,2,4-oxadiazol-3-yl)-3-ethylphenyl]oxy}butanoate (D69)

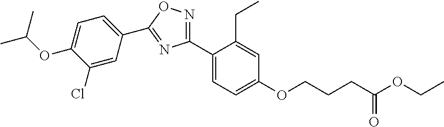

To the solution of 4-(5-{3-chloro-4-[(1-methylethyl)oxy]phenyl}-1,2,4-oxadiazol-3-yl)-3-ethylphenol (D62) (120 mg) in N,N-dimethylformamide (DMF) (2 mL) was added potassium carbonate (69.3 mg) and ethyl 4-bromobutanoate (78 mg). The resulting solution was heated to 60° C. overnight. The reaction solution was diluted with ethyl acetate (20 mL), washed with water (3*5 mL), dried over sodium sulphate, concentrated to afford ethyl 4-{[4-(5-{3-chloro-4-[(1-methylethyl)oxy]phenyl}-1,2,4-oxadiazol-3-yl)-3-ethylphenyl]oxy}butanoate (D69) (158 mg) as a crude product. MS (ES): C₂₅H₂₉ClN₂O₅ requires 472; found 473.2 (M+H⁺).

### Description for D70

### ethyl 5-{[4-(5-{3-cyano-4-[(1-methylethyl)oxy]phenyl}-1,2,4-oxadiazol-3-yl)-3-ethylphenyl]oxy}pentanoate (D70)

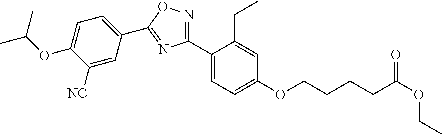

To a solution of 5-[3-(2-ethyl-4-hydroxyphenyl)-1,2,4-oxadiazol-5-yl]-2-[(1-methylethyl)oxy]benzonitrile (D65) (100 mg) in N,N-dimethylformamide (DMF) (2 mL) was added potassium carbonate (59.3 mg) and ethyl 5-bromopentanoate (65.8 mg). The reaction solution was heated to 60° C. overnight. The reaction solution was diluted with ethyl acetate (30 mL), washed with water (2*8 mL), dried over sodium sulphate, concentrated to afford ethyl 5-{[4-(5-{3-cyano-4-[(1-methylethyl)oxy]phenyl}-1,2,4-oxadiazol-3-yl)-3-ethylphenyl]oxy}pentanoate (D70) (137 mg) as a crude product. MS (ES): C₂₇H₃₁N₃O₅ requires 477; found 478.3 (M+H⁺).

### Description for D71

### ethyl 4-{[4-(5-{3-cyano-4-[(1-methylethyl)oxy]phenyl}-1,2,4-oxadiazol-3-yl)-3-ethylphenyl]oxy}butanoate (D71)

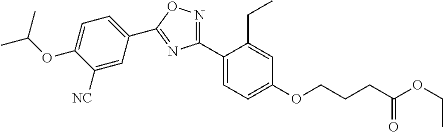

To a solution of 5-[3-(2-ethyl-4-hydroxyphenyl)-1,2,4-oxadiazol-5-yl]-2-[(1-methylethyl)oxy]benzonitrile (D65) (100 mg) in N,N-dimethylformamide (DMF) (2 mL) was added potassium carbonate (59.3 mg) and ethyl 4-bromobutanoate (61.4 mg). The reaction solution was heated to 60° C. overnight. The reaction solution was diluted with ethyl acetate (30 mL), washed with water (2*8 mL), dried over sodium sulphate, concentrated to afford ethyl 4-{[4-(5-{3-cyano-4-[(1-methylethyl)oxy]phenyl}-1,2,4-oxadiazol-3-yl)-3-ethylphenyl]oxy}butanoate (D71) (133 mg) as a crude product. MS (ES): C₂₆H₂₉N₃O₅ requires 463; found 464.2 (M+H⁺).

### Description for D72 5-(3-{4-[(2-bromoethyl)oxy]-2-ethylphenyl}-1,2,4-oxadiazol-5-yl)-2-[(1-methylethyl)oxy]benzonitrile (D72)

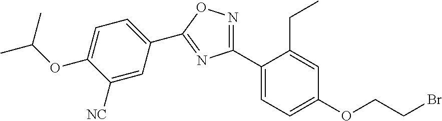

To the solution of 5-[3-(2-ethyl-4-hydroxyphenyl)-1,2,4-oxadiazol-5-yl]-2-[(1-methylethyl)oxy]benzonitrile (D65) (100 mg) in N,N-dimethylformamide (DMF) (2 mL) was added potassium carbonate (119 mg) and 1,2-dibromoethane (0.494 mL). The reaction solution was heated to 60° C. for 2 d. The reaction solution was diluted with ethyl acetate (20 mL), washed with water (3*5 mL), dried over sodium sulphate, concentrated and purified by column chromatography to afford 5-(3-{4-[(2-bromoethyl)oxy]-2-ethylphenyl}-1,2,4-oxadiazol-5-yl)-2-[(1-methylethyl)oxy]benzonitrile (D72) (110 mg) as a pale-white solid. MS (ES): C₂₂H₂₂BrN₃O₃ requires 455; found 456.2 (M+H⁺).

### Description for D73

### 3-{4-[(4-bromobutyl)oxy]-2-ethylphenyl}-5-{3-chloro-4-[(1-methylethyl)oxy]phenyl}-1,2,4-oxadiazole (D73)

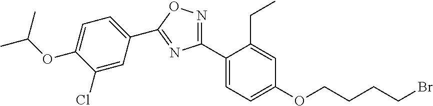

To the solution of 4-(5-{3-chloro-4-[(1-methylethyl)oxy]phenyl}-1,2,4-oxadiazol-3-yl)-3-ethylphenol (D62) (130 mg) in N,N-dimethylformamide (DMF) (2 mL) was added potassium carbonate (150 mg) and 1,4-dibromobutane (0.860 mL). The resulting solution was stirred at 60° C. overnight. The reaction solution was diluted with ethyl acetate (20 mL), washed with water (3*5 mL), dried over sodium sulphate, concentrated and purified by column chromatography to afford 3-{4-[(4-bromobutyl)oxy]-2-ethylphenyl}-5-{3-chloro-4-[(1-methylethyl)oxy]phenyl}-1,2,4-oxadiazole (D73) (120 mg). MS (ES): C₂₃H₂₆BrClN₂O₃ requires 492; found 493.2 (M+H⁺).

### Description for D74

### 3-{4-[(3-bromopropyl)oxy]-2-ethylphenyl}-5-{3-chloro-4-[(1-methylethyl)oxy]phenyl}-1,2,4-oxadiazole (D74)

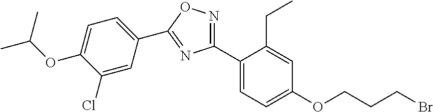

To the solution of 4-(5-{3-chloro-4-[(1-methylethyl)oxy]phenyl}-1,2,4-oxadiazol-3-yl)-3-ethylphenol (D62) (130 mg) in N,N-dimethylformamide (DMF) (2 mL) was added potassium carbonate (150 mg) and 1,3-dibromopropane (0.735 mL). The resulting solution was stirred at 60° C. overnight. The reaction solution was diluted with ethyl acetate (20 mL), washed with water (3*5 mL), dried over sodium sulphate, concentrated and purified by column chromatography to afford 3-{4-[(3-bromopropyl)oxy]-2-ethylphenyl}-5-{3-chloro-4-[(1-methylethyl)oxy]phenyl}-1,2,4-oxadiazole (D74) (120 mg). MS (ES): C₂₂H₂₄BrClN₂O₃ requires 478; found 479.1 (M+H⁺).

### Description for D75

### 5-(3-{4-[(2-bromoethyl)oxy]-2-chlorophenyl}-1,2,4-oxadiazol-5-yl)-2-[(1-methylethyl)oxy]benzonitrile (D75)

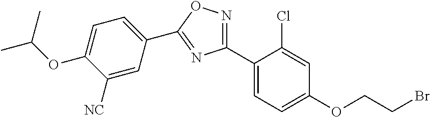

To the solution of 5-[3-(2-chloro-4-hydroxyphenyl)-1,2,4-oxadiazol-5-yl]-2-[(1-nnethylethyl)oxy]benzonitrile (D13) (100 mg) in N,N-dimethylformamide (DMF) (2 mL) was added 1,2-dibromoethane (0.485 mL) and potassium carbonate (117 mg). The reaction solution was heated to 60° C. overnight. The reaction solution was diluted with ethyl acetate (50 mL), washed with water (3*5 mL), dried over sodium sulphate, concentrated and purified by column chromatography to afford 5-(3-{4-[(2-bromoethyl)oxy]-2-chlorophenyl}-1,2,4-oxadiazol-5-yl)-2-[(1-methylethyl)oxy]benzonitrile (D75) (100 mg). MS (ES): C₂₀H₁₇BrClN₃O₃ requires 461; found 462.1 (M+H⁺).

### Description for D76

### 5-(3-{4-[(3-bromopropyl)oxy]-2-chlorophenyl}-1,2,4-oxadiazol-5-yl)-2-[(1-methylethyl)oxy]benzonitrile (D76)

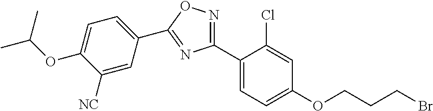

To the solution of 5-[3-(2-chloro-4-hydroxyphenyl)-1,2,4-oxadiazol-5-yl]-2-[(1-methylethyl)oxy]benzonitrile (D13) (100 mg) in N,N-dimethylformamide (DMF) (2 mL) was added 1,3-dibromopropane (0.57 mL) and potassium carbonate (117 mg). The reaction solution was heated to 60° C. overnight. The reaction solution was diluted with ethyl acetate (50 mL), washed with water (3*5 mL), dried over sodium sulphate, concentrated and purified by column chromatography to afford 5-(3-{4-[(3-bromopropyl)oxy]-2-chlorophenyl}-1,2,4-oxadiazol-5-yl)-2-[(1-methylethyl)oxy]benzonitrile (D76) (100 mg). MS (ES): C₂H₁₉BrClN₃O₃ requires 475; found 476.1 (M+H⁺).

### Description for D77

### 5-(3-{4-[(4-bromobutyl)oxy]-2-ethylphenyl}-1,2,4-oxadiazol-5-yl)-2-[(1-methylethyl)oxy]benzonitrile (D77)

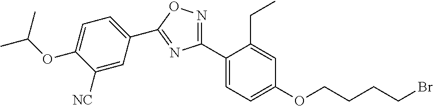

To the solution of 5-[3-(2-ethyl-4-hydroxyphenyl)-1,2,4-oxadiazol-5-yl]-2-[(1-methylethyl)oxy]benzonitrile (D65) (100 mg) in N,N-dimethylformamide (DMF) (2 mL) was added potassium carbonate (119 mg) and 1,4-dibromobutane (0.679 mL). The reaction solution was heated to 60° C. overnight. The reaction solution was diluted with ethyl acetate (10 mL), washed with water (3*5 mL), dried over sodium sulphate, concentrated and purified by column chromatography to afford 5-(3-{4-[(4-bromobutyl)oxy]-2-ethylphenyl}-1,2,4-oxadiazol-5-yl)-2-[(1-methylethyl)oxy]benzonitrile (D77) (110 mg). MS (ES): C₂₄H₂₆BrN₃O₃ requires 483; found 484.2 (M+H⁺).

### Description for D78

### 5-(3-{4-[(3-bromopropyl)oxy]-2-ethylphenyl}-1,2,4-oxadiazol-5-yl)-2-[(1-methylethyl)oxy]benzonitrile (D78)

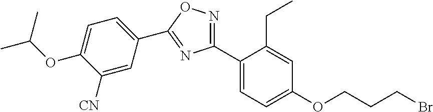

To the solution of 5-[3-(2-ethyl-4-hydroxyphenyl)-1,2,4-oxadiazol-5-yl]-2-[(1-methylethyl)oxy]benzonitrile (D65) (110 mg) in N,N-dimethylformamide (DMF) (2 mL) was added potassium carbonate (131 mg) and 1,3-dibromopropane (0.639 mL). The reaction solution was heated to 60° C. overnight. The reaction solution was diluted with ethyl acetate (10 mL), washed with water (3*5 mL), dried over sodium sulphate, concentrated and purified by column chromatography to afford 5-(3-{4-[(3-bromopropyl)oxy]-2-ethylphenyl}-1,2,4-oxadiazol-5-yl)-2-[(1-methylethyl)oxy]benzonitrile (D78) (100 mg). MS (ES): C₂₃H₂₄BrN₃O₃ requires 469; found 470.2 (M+H⁺).

### Description for D79

### 4-(5-{3-chloro-4-[(1-methylethyl)oxy]phenyl}-1,2,4-oxadiazol-3-yl)-3-methylphenol (D79)

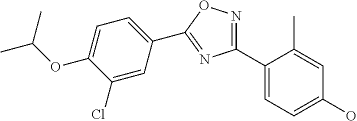

To the solution of 5-{3-chloro-4-[(1-methylethyl)oxy]phenyl}-3-{2-methyl-4-[({[2-(trimethylsilyl)ethyl]oxy}methyl)oxy]phenyl}-1,2,4-oxadiazole (D3) (2.96 g) in HMPA (10 mL) was added TBAF (8.15 g) at room temperature. The resulting solution was stirred at 40° C. for 3 h. The reaction was not all converted, so the reaction solution was stirred at room temperature for another 2 d. The reaction solution was diluted with ethyl acetate (150 ml), washed with water (3*50 ml), dried over sodium sulphate and concentrated. The residue was purified by column chromatography to give 4-(5-{3-chloro-4-[(1-methylethyl)oxy]phenyl}-1,2,4-oxadiazol-3-yl)-3-methylphenol (D79) (1.5 g) as a red-brown solid. MS (ES): C₁₈H₁₇ClN₂O₃ requires 344; found 345.1 (M+H⁺).

### Description for D80

### 3-{4-[(3-bromopropyl)oxy]-2-methylphenyl}-5-{3-chloro-4-[(1-methylethyl)oxy]phenyl}-1,2,4-oxadiazole (D80)

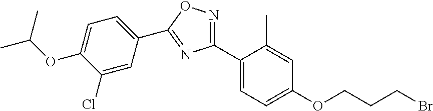

To the solution of 4-(5-{3-chloro-4-[(1-methylethyl)oxy]phenyl}-1,2,4-oxadiazol-3-yl)-3-methylphenol (D79) (150 mg) in N,N-dimethylformamide (DMF) (2 mL) was added 1,3-dibromopropane (0.883 mL) and potassium carbonate (180 mg). The reaction solution was heated to 60° C. overnight. The reaction solution was diluted with ethyl acetate (50 mL), washed with water (3*5 mL), dried over sodium sulphate, concentrated and purified by column chromatography to afford 3-{4-[(3-bromopropyl)oxy]-2-methylphenyl}-5-{3-chloro-4-[(1-methylethyl)oxy]phenyl}-1,2,4-oxadiazole (D80) (160 mg) as a white solid. MS (ES): C₂₁H₂₂BrClN₂O₃ requires 464; found 465.1 (M+H⁺).

### Description for D81

### ethyl 4-{[4-(3-bromo-1,2,4-thiadiazol-5-yl)-3-ethylphenyl]oxy}butanoate (D81)

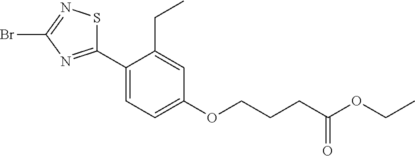

To a solution of ethyl 4-{[3-ethyl-4-(4,4,5,5-tetramethyl-1,3,2-dioxaborolan-2-yl)phenyl]oxy}butanoate (D47) (500 mg), Pd(Ph₃P)₄ (159 mg) and 3-bromo-5-chloro-1,2,4-thiadiazole (330 mg) in DME (3 mL) and water (0.5 mL) stirred under nitrogen at room temperature was added tripotassium phosphate (439 mg). The reaction vessel was sealed and heated under microwave at 120° C. for 5 h. After cooling the reaction, the mixture was evaporated and purified by column chromatography to afford ethyl 4-{[4-(3-bromo-1,2,4-thiadiazol-5-yl)-3-ethylphenyl]oxy}butanoate (D81) (130 mg). MS (ES): C₁₆H₁₉BrN₂O₃S requires 398; found 399.1 (M+H⁺).

### Description for D82

### ethyl 4-{[4-(3-{3-cyano-4-[(1-methylethyl)oxy]phenyl}-1,2,4-thiadiazol-5-yl)-3-ethylphenyl]oxy}butanoate (D82)

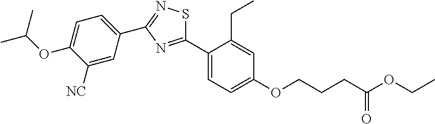

To a solution of ethyl 4-{[4-(3-bromo-1,2,4-thiadiazol-5-yl)-3-ethylphenyl]oxy}butanoate (D81) (130 mg), Pd(Ph₃P)₄ (75 mg) and 2-[(1-methylethyl)oxy]-5-(4,4,5,5-tetramethyl-1,3,2-dioxaborolan-2-yl)benzonitrile (280 mg) in N,N-dimethylformamide (DMF) (5 mL) stirred under nitrogen at room temperature was added tripotassium phosphate (207 mg) and water (1 mL). The reaction vessel was sealed and heated under microwave at 130° C. for 20 min. After cooling the reaction, the mixture was evaporated and purified by column chromatography to afford ethyl 4-{[4-(3-{3-cyano-4-[(1-methylethyl)oxy]phenyl}-1,2,4-thiadiazol-5-yl)-3-ethylphenyl]oxy}butanoate (D82) (40 mg). MS (ES): C₂₆H₂₉N₃O₄S requires 479; found 480.2 (M+H⁺).

### Description for D83

### 5-(3-bromo-1,2,4-thiadiazol-5-yl)-2-[(1-methylethyl)oxy]benzonitrile (D83)

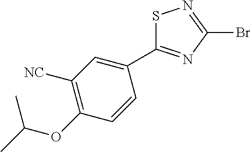

To a solution of 2-[(1-methylethyl)oxy]-5-(4,4,5,5-tetramethyl-1,3,2-dioxaborolan-2-yl)benzonitrile (280 mg), 3-bromo-5-chloro-1,2,4-thiadiazole (194 mg) and tripotassium phosphate (517 mg) in 1,2-dimethoxyethane (DME) (4 mL) and water (1 mL) under nitrogen was added Pd(Ph₃P)₄ (113 mg). The reaction vessel was sealed and heated under microwave at 120° C. for 10 min. Water was added, the reaction mixture was filtered through the celite. The aqueous layer was extracted with EA for 3 times. the combined organic layers were washed with brine, dried over anhydrous sodium sulfate. The dried solution was concentrated and purified by column chromatography to give 5-(3-bromo-1,2,4-thiadiazol-5-yl)-2-[(1-methylethyl)oxy]benzonitrile (D83) (152 mg) as a white solid. MS (ES): C₁₂H₁₀BrN₃OS requires 323; found 324.0 (M+H⁺).

### Description for D84

### ethyl 4-{[4-(5-{3-cyano-4-[(1-methylethyl)oxy]phenyl}-1,2,4-thiadiazol-3-yl)-3-ethylphenyl]oxy}butanoate (D84)

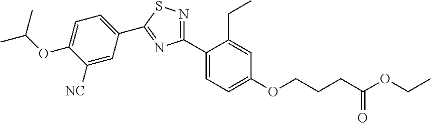

To a solution of ethyl 4-{[3-ethyl-4-(4,4,5,5-tetramethyl-1,3,2-dioxaborolan-2-yl)phenyl]oxy}butanoate (D47) (67.0 mg), Pd(Ph₃P)₄ (17.82 mg) and 5-(3-bromo-1,2,4-thiadiazol-5-yl)-2-[(1-methylethyl)oxy]benzonitrile (D83) (50 mg) in N,N-dimethylformamide (DMF) (5 mL) and water (1.000 mL) stirred under nitrogen at room temperature was added tripotassium phosphate (98 mg). The reaction vessel was sealed and heated under microwave at 120° C. for 10 min. After cooling the reaction, the mixture was evaporated and purified by column chromatography to afford ethyl 4-{[4-(5-{3-cyano-4-[(1-methylethyl)oxy]phenyl}-1,2,4-thiadiazol-3-yl)-3-ethylphenyl]oxy}

butanoate (D84) (63 mg). MS (ES): C₂₆H₂₉N₃O₄S requires 479; found 480.2 (M+H⁺).

### Description for D85

### 2-[(1-methylethyl)oxy]-5-(1,3-thiazol-2-yl)benzonitrile (D85)

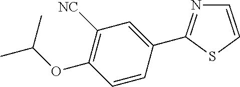

To a suspension of 2-[(1-methylethyl)oxy]-5-(4,4,5,5-tetramethyl-1,3,2-dioxaborolan-2-yl)benzonitrile (109 mg), 2-bromo-1,3-thiazole (93 mg) and cesium carbonate (148 mg) in acetonitrile (3 mL)/water (0.750 mL) stirred under nitrogen was added PdCl₂(dppf)-CH₂Cl₂ adduct (31.0 mg). The reaction vessel was sealed and heated under microwave at 120° C. for 1 h. After cooling the reaction, the reaction mixture was diluted with ethyl acetate, filtered through celite. The filtrate was partitioned between ethyl acetate and water. The organic phase was dried over sodium sulphate and evaporated in vacuo to give the crude product, which was purified by column chromatography to afford 2-[(1-methylethyl)oxy]-5-(1,3-thiazol-2-yl)benzonitrile (D85) (50 mg). MS (ES): C₁₃H₁₂N₂OS requires 244; found 245.1 (M+H⁺).

### Description for D86

### 5-(5-bromo-1,3-thiazol-2-yl)-2-[(1-methylethyl)oxy]benzonitrile (D86)

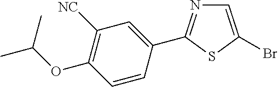

To a solution of 2-[(1-methylethyl)oxy]-5-(1,3-thiazol-2-yl)benzonitrile (D85) (215 mg) and sodium acetate (144 mg) in acetic acid (8 mL) stirred at room temperature was added a solution of Br₂ (0.045 mL) in acetic acid (1 mL) dropwise. The reaction mixture was stirred at 20° C. until start material was consumed completely. The reaction mixture was basified with 2M NaOH, then diluted with ethyl acetate. The mixture was washed with water and brine. The organic phase was dried over anhydrous sodium sulphate. After concentration, the crude product 5-(5-bromo-1,3-thiazol-2-yl)-2-[(1-methylethyl)oxy]benzonitrile (D86) (320 mg) was used for next step without further purification. δH (CDCl₃, 600 MHz): 1.36 (6H, d), 4.65 (1H, m), 6.95 (1H, d), 7.64 (1H, s), 7.92 (1H, d), 7.97 (1H, dd).

### Description for D87

### ethyl 4-{[4-(2-{3-cyano-4-[(1-methylethyl)oxy]phenyl}-1,3-thiazol-5-yl)-3-ethylphenyl]oxy}butanoate (D87)

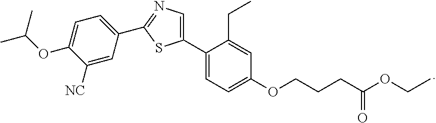

To a solution of 5-(5-bromo-1,3-thiazol-2-yl)-2-[(1-methylethyl)oxy]benzonitrile (D86) (89 mg), ethyl 4-{[3-ethyl-4-(4,4,5,5-tetramethyl-1,3,2-dioxaborolan-2-yl)phenyl]oxy}butanoate (D47) (110 mg) and Pd(Ph₃P)₄ (31.8 mg) in N,N-dimethylformamide (DMF) (5 mL) and water (1.000 mL) stirred under nitrogen at room temperature was added tripotassium phosphate (175 mg). The reaction mixture was stirred at 120° C. for 40 min. After cooling the reaction, the mixture was evaporated and purified by column chromatography to afford ethyl 4-{[4-(2-{3-cyano-4-[(1-methylethyl)oxy]phenyl}-1,3-thiazol-5-yl)-3-ethylphenyl]oxy}butanoate (D87) (59.3 mg). MS (ES): C₂₇H₃₀N₂O₄S requires 478; found 479.3 (M+H⁺).

### Description for D88

### 1,1-dimethylethyl (3-{[4-(2-{3-cyano-4-[(1-methylethyl)oxy]phenyl}-1,3-thiazol-5-yl)-3-ethylphenyl]oxy}propyl)methylcarbamate (D88)

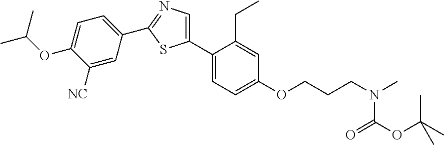

To a solution of 5-(5-bromo-1,3-thiazol-2-yl)-2-[(1-methylethyl)oxy]benzonitrile (D86) (89 mg), 1,1-dimethylethyl (3-{[3-ethyl-4-(4,4,5,5-tetramethyl-1,3,2-dioxaborolan-2-yl)phenyl]oxy}propyl)methylcarbamate (D40) (127 mg) and Pd(Ph₃P)₄ (31.8 mg) in N,N-dimethylformamide (DMF) (5 mL) and water (1.000 mL) stirred under nitrogen at room temperature was added tripotassium phosphate (175 mg). The reaction mixture was stirred at 120° C. for 30 min. After cooling the reaction, the mixture was diluted with water and extrated with ethyl acetate, the combined organic layers were dried in vacuo to afford the crude product 1,1-dimethylethyl (3-{[4-(2-{3-cyano-4-[(1-methylethyl)oxy]phenyl}-1,3-thiazol-5-yl)-3-ethylphenyl]oxy}propyl)methylcarbamate (D88) (96 mg). MS (ES): C₃₀H₃₇N₃O₄S requires 535; found 536.3 (M+H⁺).

### EXAMPLE 1

### 4-{[4-(5-{3-Chloro-4-[(1-methylethyl)oxy]phenyl}-1,2,4-oxadiazol-3-yl)-3-methylphenyl]oxy}butanoic acid (E1)

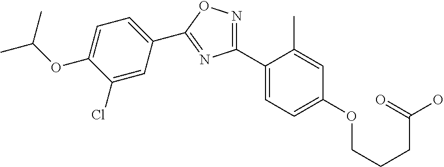

To the suspension of ethyl 4-{[4-(5-{3-chloro-4-[(1-methylethyl)oxy]phenyl}-1,2,4-oxadiazol-3-yl)-3-methylphenyl]oxy}butanoate (D4) (0.1 g) in isopropanol (5 mL) and water (5.00 mL) was added NaOH (0.872 mL, 0.5 M aq), and the resulting mixture was heated to 90° C. for 1 hour. The solvent was removed in vacuo and the residue was added water (5 mL), acidified to pH=3-4 with 2M HCl, ethyl acetate (15 mL) was added and the organic phase was separated and dried over sodium sulphate, purified by MDAP to afford 4-{[4-(5-{3-chloro-4-[(1-methylethyl)oxy]phenyl}-1,2,4-oxadiazol-3-yl)-3-methylphenyl]oxy}butanoic acid (E1) (70 mg) as a white solid. 8H (DMSO-d₆, 400 MHz): 1.35 (6H, d), 1.96 (2H, m), 2.39 (2H, t), 2.58 (3H, s), 4.06 (2H, t), 4.87 (1H, m), 6.96 (2H, m), 7.42 (1H, d), 7.97 (1H, d), 8.07 (1H, dd), 8.15 (1H, d), 12.13 (1H, s). MS (ES): C₂₂H₂₃ClN₂O₅ requires 430; found 431.2 (M+H⁺).

### EXAMPLE 2

### 5-{[4-(5-{3-Chloro-4-[(1-methylethyl)oxy]phenyl}-1,2,4-oxadiazol-3-yl)-3-methylphenyl]oxy}pentanoic acid (E2)

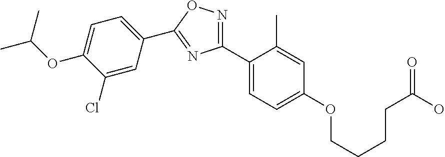

To the suspension of ethyl 5-{[4-(5-{3-chloro-4-[(1-methylethyl)oxy]phenyl}-1,2,4-oxadiazol-3-yl)-3-methylphenyl]oxy}pentanoate (D5) (0.1 g) in isopropanol (5 mL) and water (5.00 mL) was added NaOH (0.85 mL, 0.5 M aq), and the resulting mixture was heated to 90° C. for 1 hour. The solvent was removed in vacuo and the residue was added water (5 mL), acidified to pH=3-4 with 2M HCl, ethyl acetate (15 mL) was added and the organic phase was separated and dried over sodium sulphate, purified by MDAP to afford 5-{[4-(5-{3-chloro-4-[(1-methylethyl)oxy]phenyl}-1,2,4-oxadiazol-3-yl)-3-methylphenyl]oxy}pentanoic acid (E2) (35 mg) as a white solid. δH (DMSO-d₆, 400 MHz): 1.35 (6H, d), 1.70 (4H, m), 2.29 (2H, t), 2.57 (3H, s), 4.04 (2H, t), 4.86 (1H, m), 6.95 (2H, m), 7.41 (1H, d), 7.96 (1H, d), 8.06 (1H, dd), 8.14 (1H, d), 12.02 (1H, s). MS (ES): C₂₃H₂₆ClN₂O₆ requires 444; found 445.2 (M+H⁺).

### EXAMPLE 3

### {[4-(5-{3-Chloro-4-[(1-methylethyl)oxy]phenyl}-1,2,4-oxadiazol-3-yl)-3-methylphenyl]oxy}acetic acid (E3)

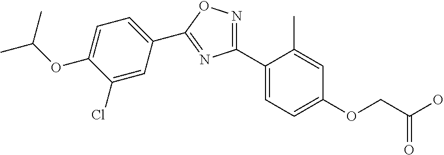

To the suspension of ethyl {[4-(5-{3-chloro-4-[(1-methylethyl)oxy]phenyl}-1,2,4-oxadiazol-3-yl)-3-methylphenyl]oxy}acetate (D6) (100 mg) in isopropanol (5 mL) and water (5.00 mL) was added NaOH (0.93 mL, 0.5 M aq), and the resulting mixture was heated to 90° C. for 1 hour. The solvent was removed in vacuo and the residue was added water (5 mL), acidified to pH=3-4 with 2M HCl, ethyl acetate (15 mL) was added and the organic phase was separated and dried over sodium sulphate, purified by MDAP to afford {[4-(5-{3-chloro-4-[(1-methylethyl)oxy]phenyl}-1,2,4-oxadiazol-3-yl)-3-methylphenyl]oxy}acetic acid (E3) (45 mg) as a white solid. δH (DMSO-d₆, 400 MHz): 1.35 (6H, d), 2.58 (s, 3H), 4.76 (2H, s), 4.86 (1H, m), 6.94 (1H, dd), 6.98 (1H, d), 7.42 (1H, d), 7.96 (1H, d), 8.08 (1H, dd), 8.15 (1H, d), 13.04 (1H, s). MS (ES): C₂₀H₁₉ClN₂O₅ requires 402; found 403.1 (M+H⁺).

### EXAMPLE 4

### 3-{[4-(5-{3-Chloro-4-[(1-methylethyl)oxy]phenyl}-1,2,4-oxadiazol-3-yl)-3-methylphenyl]oxy}-N,N-dimethyl-1-propanamine (E4)

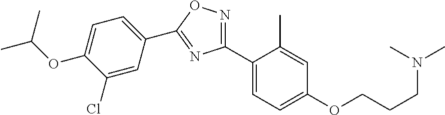

To the solution of 4-(5-{3-chloro-4-[(1-methylethyl)oxy]phenyl}-1,2,4-oxadiazol-3-yl)-3-methylphenol (80 mg) in tetrahydrofuran (5 mL) was added Ph₃P (183 mg), 3-(dimethylamino)-1-propanol (0.082 mL). Then DIAD (0.135 mL) was added at room temp. The resulting solution was stirred at reflux for overnight. The resulting solution was concentrated and purified by MDAP to offord 3-{[4-(5-{3-chloro-4-[(1-methylethyl)oxy]phenyl}-1,2,4-oxadiazol-3-yl)-3-methylphenyl]oxy}-N,N-dimethyl-1-propanamine (E4) (11 mg) as a off-white solid. δH (DMSO-d₆, 400 MHz): 1.35 (6H, d), 2.12 (2H, m), 2.59 (3H, s), 2.82 (6H, s), 3.23 (2H, s), 4.13 (2H, t), 4.87 (1H, m), 6.97 (2H, m), 7.43 (1H, d), 7.99 (1H, d), 8.08 (1H, dd), 8.15 (1H, d). MS (ES): C₂₃H₂₅ClN₃O₃ requires 429; found 430.2 (M+H⁺).

### EXAMPLE 5

### 2-{[4-(5-{3-Chloro-4-[(1-methylethyl)oxy]phenyl}-1,2,4-oxadiazol-3-yl)-3-methylphenyl]oxy}-N-methylethanamine (E5)

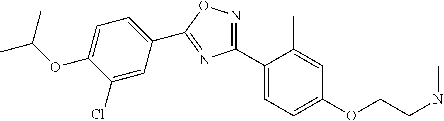

To the solution of 3-{4-[(2-bromoethyl)oxy]-2-methylphenyl}-5-{3-chloro-4-[(1-methylethyl)oxy]phenyl}-1,2,4-oxadiazole (D7) (50 mg) in tetrahydrofuran (2 mL) was added methylamine (0.553 mL, 2M in THF). The resulting solution was stirred at 60° C. for overnight. The solvent was removed and the residue was purified by MDAP to afford 2-{[4-(5-{3-chloro-4-[(1-methylethyl)oxy]phenyl}-1,2,4-oxadiazol-3-yl)-3-methylphenyl]oxy}-N-methylethanamine (E5) (22 mg) as an oil. 2M HCl in methanol (0.5 mL) was added to the methanol solution, after removing the solvent, HCl salt solid was obtained as a solid. δH (DMSO-d₆, 400 MHz): 1.35 (6H, d), 2.61 (6H, m), 3.37 (2H, s), 4.34 (2H, s), 4.86 (1H, m), 7.03 (2H, m), 7.43 (1H, d), 8.01 (1H, d), 8.07 (1H, d), 8.15 (1H, s), 9.11 (1H, s). MS (ES): C₂₁H₂₄ClN₃O₃-requires 401; found 402.2 (M+H⁺).

### EXAMPLE 6

### 5-{3-Chloro-4-[(1-methylethyl)oxy]phenyl}-3-(2-methyl-4-{[2-(methyloxy)ethyl]oxy}phenyl)-1,2,4-oxadiazole (E6)

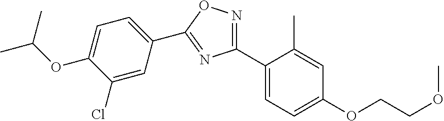

To the solution of 3-chloro-4-[(1-methylethyl)oxy]benzoic acid (118 mg) in THF (15 mL) was added EDC (150 mg), HOBt (120 mg) and the resulting solution was stirred at RT for 2 hours. To the reaction solution was added N-hydroxy-2-methyl-4-{[2-(methyloxy)ethyl]oxy}benzenecarboximidamide (D9) (117 mg) and the suspension was stirred at RT for another 2 hours. THF was removed in vacuo and the residue was dissolved in ethyl acetate (100 mL), washed with water (2×20 mL), dried over sodium sulphate, concentrated. The residue in dioxane (50 mL) was heated to reflux for 4 hours. Dioxane was removed in vacuo and the residue was purified by column to afford 5-{3-chloro-4-[(1-methylethyl)oxy]phenyl}-3-(2-methyl-4-{[2-(methyloxy)ethyl]oxy}phenyl)-1,2,4-oxadiazole (E6) (80 mg). δH (CDCl₃, 400 MHz): 1.46 (6H, d), 2.67 (3H, s), 3.48 (3H, s), 3.79 (2H, m), 4.20 (2H, m), 4.72 (1H, m), 6.89 (2H, m), 7.06 (1H, d), 8.05 (2H, m), 8.24 (1H, d). MS (ES): C₂₁H₂₃ClN₂O₄ requires 402; found 403.2 (M+H⁺).

### EXAMPLE 7

### 5-{[3-Chloro-4-(5-{3-cyano-4-[(1-methylethyl)oxy]phenyl}-1,2,4-oxadiazol-3-yl)phenyl]oxy}pentanoic acid (E7)

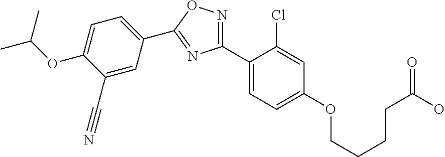

To the suspension of ethyl 5-{[3-chloro-4-(5-{3-cyano-4-[(1-methylethyl)oxy]phenyl}-1,2,4-oxadiazol-3-yl)phenyl]oxy}pentanoate (D14) (140 mg) in isopropanol (10 mL) and water (10.00 mL) was added NaOH (2.89 mL, 0.5 M aq), and the resulting mixture was heated to 90° C. for 2 hours. The solvent was removed in vacuo and the residue was added water (10 mL), acidified to pH=1-2 with 2M HCl, ethyl acetate (50 mL) was added and the organic phase was separated and dried over sodium sulphate, purified by MDAP to afford 5-{[3-chloro-4-(5-{3-cyano-4-[(1-methylethyl)oxy]phenyl}-1,2,4-oxadiazol-3-yl)phenyl]oxy}pentanoic acid (E7) (50 mg) as a white solid. δH (DMSO-d₆, 400 MHz): 1.37 (6H, d), 1.71 (4H, m), 2.30 (2H, t), 4.09 (2H, t), 4.96 (1H, m), 7.12 (1H, dd), 7.24 (1H, d), 7.53 (1H, d), 7.94 (1H, d), 8.37 dd), 8.47 (1H, d), 12.08 (1H, s). MS (ES): C₂₃H₂₂ClN₃O₅ requires 455; found 456.2 (M+H⁺).

### EXAMPLE 8

### 5-{[4-(5-{3-Cyano-4-[(1-methylethyl)oxy]phenyl}-1,2,4-oxadiazol-3-yl)phenyl]oxy}pentanoic acid (E8)

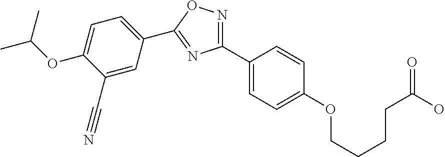

To the suspension of ethyl 5-{[4-(5-{3-cyano-4-[(1-methylethyl)oxy]phenyl}-1,2,4-oxadiazol-3-yl)phenyl]oxy}pentanoate (D19) (160 mg) in isopropanol (8 mL) and water (8.00 mL) was added NaOH (3.56 mL, 0.5 M aq), and the resulting mixture was heated to 90° C. for 2 hours. The solvent was removed in vacuo and the residue was added water (10 mL), acidified to pH=1-2 with 2M HCl, ethyl acetate (50 mL) was added and the organic phase was separated and dried over sodium sulphate, purified by MDAP to afford 5-{[4-(5-{3-cyano-4-[(1-methylethyl)oxy]phenyl}-1,2,4-oxadiazol-3-yl)phenyl]oxy}pentanoic acid (E8) (50 mg) as a white solid. δH (DMSO-d₆, 400 MHz): 1.37 (6H, d), 1.70 (4H, m,), 2.29 (2H, t), 4.06 (2H, t), 4.95 (1H, m), 7.11 (2H, d), 7.52 (1H, d), 7.98 (2H, d), 8.37 (1H, dd), 8.46 (1H, d), 12.05 (1H, s). MS (ES): C₂₃H₂₃N₃O₅ requires 421; found 422.2 (M+H⁺).

### EXAMPLE 9

### {[3-Chloro-4-(5-{3-cyano-4-[(1-methylethyl)oxy]phenyl}-1,2,4-oxadiazol-3-yl)phenyl]oxy}acetic acid (E9)

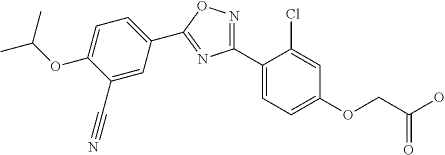

To the suspension of ethyl {[3-chloro-4-(5-{3-cyano-4-[(1-methylethyl)oxy]phenyl}-1,2,4-oxadiazol-3-yl)phenyl]oxy}acetate (D20) (90 mg) in isopropanol (8 mL) and water (8.00 mL) was added NaOH (2.04 mL, 0.5 M aq), and the resulting mixture was heated to 90° C. for 2 hours. The solvent was removed in vacuo and the residue was added water (10 mL), acidified to pH=1-2 with 2M HCl, ethyl acetate (50 mL) was added and the organic phase was separated and dried over sodium sulphate, purified by MDAP to afford {[3-chloro-4-(5-{3-cyano-4-[(1-methylethyl)oxy]phenyl}-1,2,4-oxadiazol-3-yl)phenyl]oxy}acetic acid (E9) (47 mg) as a white solid. δH (DMSO-d₆, 400 MHz): 1.32 (6H, d), 4.80 (2H, s), 4.91 (1H, m), 7.07 (1H, dd), 7.21 (1H, d), 7.49 (1H, d), 7.89 (1H, d), 8.32 (1H, dd), 8.43 (1H, d). MS (ES): C₂₀H₁₆ClN₃O₅ requires 413; found 414.1 (M+H⁺).

### EXAMPLE 10

### 4-{[3-Chloro-4-(5-{3-cyano-4-[(1-methylethyl)oxy]phenyl}-1,2,4-oxadiazol-3-yl)phenyl]oxy}butanoic acid (E10)

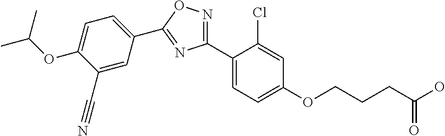

To the suspension of ethyl 4-{[3-chloro-4-(5-{3-cyano-4-[(1-methylethyl)oxy]phenyl}-1,2,4-oxadiazol-3-yl)phenyl]oxy}butanoate (D21) (157 mg) in isopropanol (8 mL) and water (8.00 mL) was added NaOH (3.34 mL, 0.5 M aq), and the resulting mixture was heated to 90° C. for 2 hours. The solvent was removed in vacuo and the residue was added water (10 mL), acidified to pH=1-2 with 2M HCl, ethyl acetate (50 mL) was added and the organic phase was separated and dried over sodium sulphate, purified by MDAP to afford 4-{[3-chloro-4-(5-{3-cyano-4-[(1-methylethyl)oxy]phenyl}-1,2,4-oxadiazol-3-yl)phenyl]oxy}butanoic acid (E10) (44 mg) as a white solid. 6H (DMSO-d₆, 400 MHz): 1.32 (6H, d), 1.91 (2H, m), 2.34 (2H, t), 4.06 (2H, t), 4.91 (1H, m), 7.07 (1H, dd), 7.20 (1H, d), 7.48 (1H, d), 7.90 (1H, d), 8.32 (1H, dd), 8.42 (1H, d), 12.13 (1H, s). MS (ES): C₂₂H₂₀ClN₃O₅ requires 441; found 442.2 (M+111.

### EXAMPLE 11

### 5-{[4-(5-{3-Cyano-4-[(1-methylethyl)oxy]phenyl}-1,2,4-oxadiazol-3-yl)-3-fluorophenyl]oxy}pentanoic acid (E11)

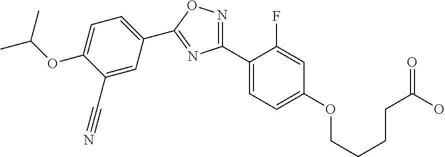

To the suspension of ethyl 5-{[4-(5-{3-cyano-4-[(1-methylethyl)oxy]phenyl}-1,2,4-oxadiazol-3-yl)-3-fluorophenyl]oxy}pentanoate (D26) (100 mg) in isopropanol (8 mL) and water (8.00 mL) was added NaOH (2.14 mL, 0.5 M aq), and the resulting mixture was heated to 90° C. for 2 hours. The solvent was removed in vacuo and the residue was added water (10 mL), acidified to pH=1-2 with 2M HCl, ethyl acetate (50 mL) was added and the organic phase was separated and dried over sodium sulphate, purified by MDAP to afford 5-{[4-(5-{3-cyano-4-[(1-methylethyl)oxy]phenyl}-1,2,4-oxadiazol-3-yl)-3-fluorophenyl]oxy}pentanoic acid (E11) (78 mg) as a white solid. δH (DMSO-d₆, 400 MHz): 1.32 (6H, d), 1.69 (4H, m), 2.24 (2H, t), 4.03 (2H, t), 4.90 (1H, m), 6.93 (1H, d), 7.01 (1H, d), 7.47 (1H, d), 7.95 (1H, m), 8.31 (1H, d), 8.41 (1H, s), 12.02 (1H, s). MS (ES): C₂₃H₂₂FN₃O₅ requires 439; found 440.2 (M+H⁺).

### EXAMPLE 12

### 5-{[4-(5-{3-Cyano-4-[(1-methylethyl)oxy]phenyl}-1,2,4-oxadiazol-3-yl)-3-(methyloxy)phenyl]oxy}pentanoic acid (E12)

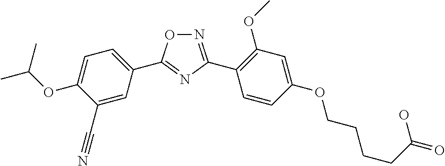

To the suspension of ethyl 5-{[4-(5-{3-cyano-4-[(1-methylethyl)oxy]phenyl}-1,2,4-oxadiazol-3-yl)-3-(methyloxy)phenyl]oxy}pentanoate (D32) (200 mg) in isopropanol (8 mL) and water (8.00 mL) was added NaOH (4.17 mL, 0.5 M aq), and the resulting mixture was heated to 90° C. for 2 hours. The solvent was removed in vacuo and the residue was added water (10 mL), acidified to pH=1-2 with 2M HCl, ethyl acetate (50 mL) was added and the organic phase was separated and dried over sodium sulphate, purified by MDAP to afford 5-{[4-(5-{3-cyano-4-[(1-methylethyl)oxy]phenyl}-1,2,4-oxadiazol-3-yl)-3-(methyloxy)phenyl]oxy}pentanoic acid (E12) (70 mg) as a white solid. δH (DMSO-d₆, 400 MHz): 1.31 (6H, d), 1.69 (4H, m), 2.23 (2H, m), 3.82 (3H, s), 4.02 (2H, t), 4.90 (1H, m), 6.66 (2H, m), 7.47 (1H, d), 7.84 (1H, d), 8.30 (1H, dd), 8.40 (1H, d), 12.01 (1H, br s). MS (ES): C₂₄H₂₅N₃O₆ requires 451; found 452.3 (M+H⁺).

### EXAMPLE 13

### 5-[5-(2-ethyl-4-{[3-(methylamino)propyl]oxy}phenyl)-1,3,4-thiadiazol-2-yl]-2-[(1-methylethyl)oxy]benzonitrile (E13)

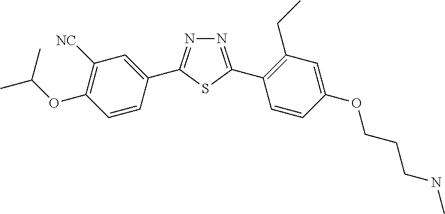

To a solution of 1,1-dimethylethyl (3-{[4-(5-{3-cyano-4-[(1-methylethyl)oxy]phenyl}-1,3,4-thiadiazol-2-yl)-3-ethylphenyl]oxy}propyl)methylcarbamate (D41) (120 mg) in dichloromethane (DCM) (20 mL) was added TFA (0.345 mL). The reaction solution was stirred at room temp for 3 h. Sat. sodium bicarbonate was added and after separation, the aqueous layer was extracted with DCM (10 mL) and the combined organic phases were dried over sodium sulphate, concentrated and purified by column chromatography to afford 5-[5-(2-ethyl-4-{[3-(methylamino)propyl]oxy}phenyl)-1,3,4-thiadiazol-2-yl]-2-[(1-methylethyl)oxy]benzonitrile (E13) (32 mg) as a white solid. δH (DMSO-d₆, 400 MHz): 1.18 (3H, t), 1.37 (6H, d), 2.08 (2H, m), 2.62 (3H, s), 2.92 (2H, m), 3.09 (2H, s), 4.16 (2H, t), 4.93 (1H, m), 6.99 (2H, m), 7.49 (1H, d), 7.65 (1H, d), 8.28 (1H, dd), 8.35 (1H, d), 8.58 (2H, br s). δF (DMSO-d₆, 375 MHz): −73.5. MS (ES): C₂₄H₂₈N₄O₂S requires 436; found 437.2 (M+H⁺).

### EXAMPLE 14

### 5-[5-(2-ethyl-4-{[3-(methylamino)propyl]oxy}phenyl)-1,3,4-oxadiazol-2-yl]-2-[(1-methylethyl)oxy]benzonitrile (E14)

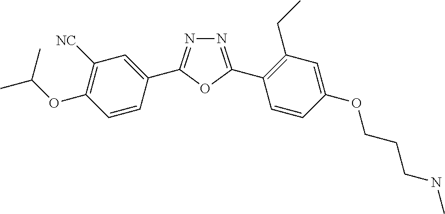

To a solution of 1,1-dimethylethyl (3-{[4-(5-{3-cyano-4-[(1-methylethyl)oxy]phenyl}-1,3,4-oxadiazol-2-yl)-3-ethylphenyl]oxy}propyl)methylcarbamate (D44) (80 mg) in dichloromethane (DCM) (10 mL) was added TFA (0.237 mL). The reaction solution was stirred at room temp for 3 h. Sat, sodium bicarbonate was added and after separation, the aqueous layer was extracted with DCM (10 mL) and the combined organic phases were dried over sodium sulphate, concentrated and purified by column chromatography to afford 5-[5-(2-ethyl-4-{[3-(methylamino)propyl]oxy}phenyl)-1,3,4-oxadiazol-2-yl]-2-[(1-methylethyl)oxy]benzonitrile (E14) (28 mg) as a white solid. δH (DMSO-d₆, 400 MHz): 1.23 (3H, t), 1.38 (6H, d), 2.08 (2H, m), 2.62 (3H, t), 3.09 (4H, m), 4.17 (2H, t), 4.95 (1H, m), 7.01 (2H, m), 7.53 (1H, d), 8.08 (1H, d), 8.32 (1H, dd), 8.44 (3H, m). δF (DMSO-d₆, 376 MHz): −73.8. MS (ES): C₂₄H₂₅N₄O₃ requires 420; found 421.3 (M+H⁺).

### EXAMPLE 15

### 4-{[4-(5-{3-cyano-4-[(1-methylethyl)oxy]phenyl}-1,3,4-oxadiazol-2-yl)-3-ethylphenyl]oxy}butanoic acid (E15)

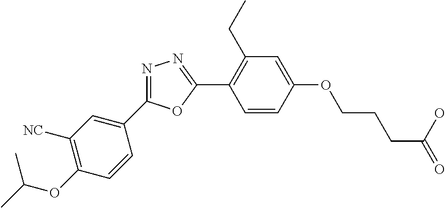

To a solution of ethyl 4-{[4-(5-{3-cyano-4-[(1-methylethyl)oxy]phenyl}-1,3,4-oxadiazol-2-yl)-3-ethylphenyl]oxy}butanoate (D48) (128 mg) in isopropanol (5 mL) and water (5.00 mL) was added NaOH (2.76 mL). The reaction solution was heated to 90° C. for 2 h. The solvent was removed in vacuo and the residue was diluted with water (10 mL), acidified by HCl (2N) to pH=2-3, extracted with ethyl acetate (2*15 mL). The combined organic phases were dried over sodium sulphate, concentrated and purified by Mass Directed Auto Prep to afford 4-{[4-(5-{3-cyano-4-[(1-methylethyl)oxy]phenyl}-1,3,4-oxadiazol-2-yl)-3-ethylphenyl]oxy}butanoic acid (E15) (25 mg) as a white solid. δH (DMSO-d₆, 400 MHz): 1.19 (3H, t), 1.36 (6H, d), 1.97 (2H, m), 2.40 (2H, t), 3.07 (2H, q), 4.08 (2H, t), 4.93 (1H, m), 6.98 (2H, m), 7.50 (1H, d), 8.02 (1H, d), 8.29 (111, dd), 8.41 (1H, d), 12.17 (1H, br s). MS (ES): C₂₄H₂₅N₃O₅ requires 435; found 436.2 (M+H⁺).

### EXAMPLE 16

### 4-{[4-(5-{3-cyano-4-[(1-methylethyl)oxy]phenyl}-1,3,4-thiadiazol-2-yl)-3-ethylphenyl]oxy}butanoic acid (E16)

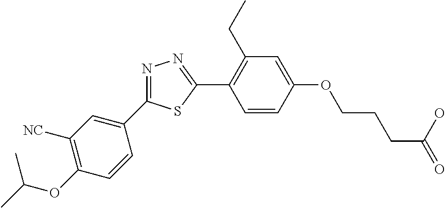

To a solution of ethyl 4-{[4-(5-{3-cyano-4-[(1-methylethyl)oxy]phenyl}-1,3,4-thiadiazol-2-yl)-3-ethylphenyl]oxy}butanoate (D49) (50 mg) in isopropanol (5 mL) and water (5.00 mL) was added 0.5 M NaOH (1.043 mL). The reaction solution was heated to 90° C. for 2 h. The solvent was removed in vacuo and the residue was diluted with water (10 mL), acidified by HCl (2N) to pH=2-3, extracted with ethyl acetate (2*15 mL). The combined organic phases were dried over sodium sulphate, concentrated and purified by Mass Directed Auto Prep to afford 4-{[4-(5-{3-cyano-4-[(1-methylethyl)oxy]phenyl}-1,3,4-thiadiazol-2-yl)-3-ethylphenyl]oxy}butanoic acid (E16) (18 mg) as a white solid. δH (DMSO-d₆, 400 MHz): 1.10 (3H, t), 1.30 (6H, d), 1.90 (2H, m), 2.34 (2H, t), 2.85 (2H, q), 4.02 (2H, t), 4.87 (1H, m), 6.92 (2H, m), 7.41 (1H, d), 7.55 (1H, d), 8.21 (1H, dd), 8.29 (1H, d). δF (DMSO-d₆, 376 MHz): −73.4. MS (ES): C₂₄H₂₅N₃O₄S requires 451; found 452.2 (WW).

### EXAMPLE 17

### 5-[3-(2-chloro-4-{[4-(dimethylamino)butyl]oxy}phenyl)-1,2,4-oxadiazol-5-yl]-2-[(1-methylethyl)oxy]benzonitrile (E17)

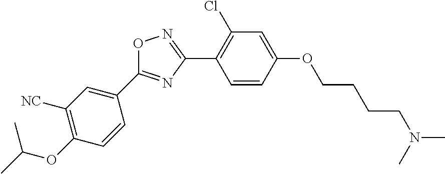

To a solution of 5-(3-{4-[(4-bromobutyl)oxy]-2-chlorophenyl}-1,2,4-oxadiazol-5-yl)-2-[(1-methylethyl)oxy]benzonitrile (D50) (80 mg) in tetrahydrofuran (THF) (2 mL) was added potassium carbonate (496 mg) and hydrogen chloride —N-methylmethanamine (1:1) (266 mg). The reaction solution was stirred in a sealed tube at 35° C. overnight. After filtration, the filtrate was concentrated and purified by Mass Directed Auto Prep to afford 5-[3-(2-chloro-4-{[4-(dimethylamino)butyl]oxy}phenyl)-1,2,4-oxadiazol-5-yl]-2-[(1-methylethyl)oxy]benzonitrile (E17) (14 mg) as a white solid. δH (DMSO-d₆, 400 MHz): 1.31 (6H, d), 1.71 (4H, m), 2.72 (6H, s), 3.06 (2H, s), 4.08 (2H, s), 4.91 (1H, m), 7.07 (1H, dd), 7.21 (1H, d), 7.49 (1H, d), 7.91 (1H, d), 8.31 (1H, dd), 8.43 (1H, d), 9.33 (1H, br s). δF (DMSO-d₆, 376 MHz): −73.5. MS (ES): C₂₄H₂₇ClN₄O₃ requires 454; found 455.3 (M+H⁺).

### EXAMPLE 18

### 5-{3-[2-chloro-4-({4-[(1-methylethyl)amino]butyl}oxy)phenyl]-1,2,4-oxadiazol-5-yl}-2-[(1-methylethyl)oxy]benzonitrile (E18)

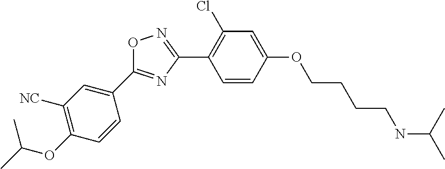

To a solution of 5-(3-{4-[(4-bromobutyl)oxy]-2-chlorophenyl}-1,2,4-oxadiazol-5-yl)-2-[(1-methylethyl)oxy]benzonitrile (D50) (80 mg) in tetrahydrofuran (THF) (2 mL) was added 2-propanamine (0.5 mL). The reaction solution was stirred in a sealed tube at 35° C. overnight. After filtration, the filtrate was concentrated and purified by Mass Directed Auto Prep to afford 5-{3-[2-chloro-4-({4-[(1-methylethyl)amino]butyl}oxy)phenyl]-1,2,4-oxadiazol-5-yl}-2-[(1-methylethyl)oxy]benzonitrile (E18) (23 mg) as a white solid. δH (DMSO-d₆, 400 MHz): 1.12 (6H, d), 1.32 (6H, d), 1.73 (4H, m), 2.91 (2H, m), 3.20 (1H, m), 4.09 (2H, t), 4.90 (1H, m), 7.07 (1H, dd), 7.21 (1H, d), 7.49 (1H, d), 7.91 (1H, d), 8.30 (3H, m br), 8.43 (1H, d). δF (DMSO-d₆, 376 MHz): −73.5. MS (ES): C₂₆H₂₆ClN₄O₃ requires 468; found 469.3 (M+H⁺).

### EXAMPLE 19

### 5-[3-(2-chloro-4-{[4-(propylamino)butyl]oxy}phenyl)-1,2,4-oxadiazol-5-yl]-2-[(1-methylethyl)oxy]benzonitrile (E19)

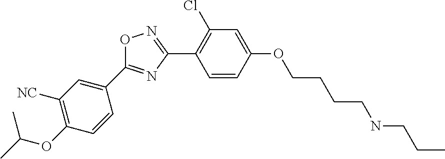

To a solution of 5-(3-{4-[(4-bromobutyl)oxy]-2-chlorophenyl}-1,2,4-oxadiazol-5-yl)-2-[(1-methylethyl)oxy]benzonitrile (D50) (80 mg) in tetrahydrofuran (THF) (2 mL) was added propylamine (0.5 mL). The reaction solution was stirred in a sealed tube at 35° C. overnight. After filtration, the filtrate was concentrated and purified by Mass Directed Auto Prep to afford 5-[3-(2-chloro-4-{[4-(propylamino)butyl]oxy}phenyl)-1,2,4-oxadiazol-5-yl]-2-[(1-methylethyl)oxy]benzonitrile (E19) (52 mg) as a white solid. δH (DMSO-d₆, 400 MHz): 0.84 (3H, t), 1.32 (6H, d), 1.54 (2H, m), 1.72 (4H, m), 2.80 (2H, t), 2.91 (2H, s), 4.08 (2H, t), 4.91 (1H, m), 7.07 (1H, dd), 7.21 (1H, d), 7.49 (1H, d), 7.91 (1H, d), 8.32 (3H, m br), 8.43 (1H, d). δF (DMSO-d₆, 376 MHz): −73.5. MS (ES): C₂₅H₂₉ClN₄O₃ requires 468; found 469.2 (M+H⁺).

### EXAMPLE 20

### 5-[3-(2-chloro-4-{[4-(ethylamino)butyl]oxy}phenyl)-1,2,4-oxadiazol-5-yl]-2-[(1-methylethyl)oxy]benzonitrile (E20)

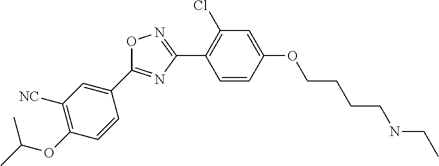

To a solution of 5-(3-{4-[(4-bromobutyl)oxy]-2-chlorophenyl}-1,2,4-oxadiazol-5-yl)-2-[(1-methylethyl)oxy]benzonitrile (D50) (80 mg) in tetrahydrofuran (THF) (2 mL) was added potassium carbonate (496 mg) and hydrogen chloride-ethanamine (1:1) (266 mg). The reaction solution was stirred in a sealed tube at 35° C. overnight. After filtration, the filtrate was concentrated and purified by Mass Directed Auto Prep to afford 5-[3-(2-chloro-4-{[4-(ethylamino)butyl]oxy}phenyl)-1,2,4-oxadiazol-5-yl]-2-[(1-methylethyl)oxy]penzonitrile (E20) (39 mg) as a white solid. δH (DMSO-d₆, 400 MHz): 1.11 (3H, t), 1.32 (6H, d), 1.72 (4H, m), 2.90 (4H, m), 4.08 (2H, t), 4.91 (1H, m), 7.07 (1H, dd), 7.21 (1H, d), 7.49 (1H, d), 7.91 (1H, d), 8.32 (3H, m br), 8.43 (1H, d). δF (DMSO-d₆, 376 MHz): −73.5. MS (ES): C₂₄H₂₇ClN₄O₃ requires 454; found 455.2 (M+H⁺).

### EXAMPLE 21

### 5-[3-(2-chloro-4-{[4-(methylamino)butyl]oxy}phenyl)-1,2,4-oxadiazol-5-yl]-2-[(1-methylethyl)oxy]benzonitrile (E21)

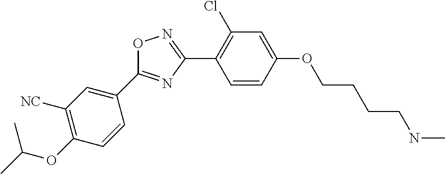

To a solution of 5-(3-{4-[(4-bromobutyl)oxy]-2-chlorophenyl}-1,2,4-oxadiazol-5-yl)-2-[(1-methylethyl)oxy]benzonitrile (D50) (80 mg) in tetrahydrofuran (THF) (2 mL) was added methylamine (2 mL). The reaction solution was stirred in a sealed tube at 35° C. overnight. After filtration, the filtrate was concentrated and purified by Mass Directed Auto Prep to afford 5-[3-(2-chloro-4-{[4-(methylamino)butyl]oxy}phenyl)-1,2,4-oxadiazol-5-yl]-2-[(1-methylethyl)oxy]benzonitrile (E21) (53 mg) as a white solid. δH (DMSO-d₆, 400 MHz): 1.32 (6H, d), 1.70 (4H, m), 2.51 (3H, s), 2.91 (2H, s), 4.07 (2H, t), 4.91 (1H, m), 7.07 (1H, dd), 7.21 (1H, d), 7.49 (1H, d), 7.91 (1H, d), 8.33 (3H, m br), 8.43 (1H, d). δF (DMSO-d₆, 376 MHz): −73.5. MS (ES): C₂₃H₂₆ClN₄O₃ requires 440; found 441.2 (M+H⁺).

### EXAMPLE 22

### 4-{[4-(5-{3-cyano-4-[(1-methylethyl)oxy]phenyl}-1,2,4-oxadiazol-3-yl)-3-fluorophenyl]oxy}butanoic acid (E22)

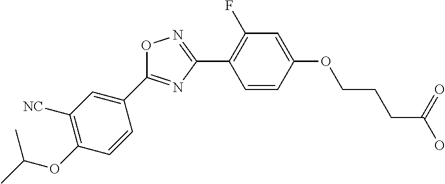

To a solution of ethyl 4-{[4-(5-{3-cyano-4-[(1-methylethyl)oxy]phenyl}-1,2,4-oxadiazol-3-yl)-3-fluorophenyl]oxy}butanoate (D51) (120 mg) in isopropanol (5 mL) and water (5.00 mL) was added 0.5 M NaOH (2.65 mL). The reaction solution was heated to 90° C. for 2 h. Isopropanol was removed in vacuo and the residue was acidified to pH=2-3, then extracted with ethyl acetate (3*10 mL). The combined organic phases were dried over sodium sulphate, concentrated and purified by Mass Directed Auto Prep to afford 4-{[4-(5-{3-cyano-4-[(1-methylethyl)oxy]phenyl}-1,2,4-oxadiazol-3-yl)-3-fluorophenyl]oxy}butanoic acid (E22) (37 mg) as a white solid. δH (DMSO-d₆, 400 MHz): 1.42 (6H, d), 2.02 (2H, m), 2.45 (2H, t), 4.14 (2H, t), 4.99 (1H, m), 7.07 (2H, m), 7.55 (1H, d), 8.03 (1H, m), 8.38 (1H, dd), 8.46 (1H, d), 12.22 (1H, br s). SF (DMSO-d₆, 376 MHz): −105.6, −73.4. MS (ES): C₂₂H₂₀FN₃O₆ requires 425; found 426.2 (M+H⁺).

### EXAMPLE 23

### {[4-(5-{3-cyano-4-[(1-methylethyl)oxy]phenyl}-1,2,4-oxadiazol-3-yl)-3-fluorophenyl]oxy}acetic acid (E23)

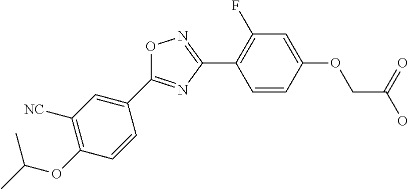

To a solution of ethyl {[4-(5-{3-cyano-4-[(1-methylethyl)oxy]phenyl}-1,2,4-oxadiazol-3-yl)-3-fluorophenyl]oxy}acetate (D52) (120 mg) in isopropanol (5 mL) and water (5.00 mL) was added 0.5 M NaOH (2.82 mL). The reaction solution was heated to 90° C. for 2 h. Isopropanol was removed in vacuo and the residue was acidified to pH=2-3, then extracted with ethyl acetate (3*10 mL). The combined organic phases were dried over sodium sulphate, concentrated and purified by Mass Directed Auto Prep to afford {[4-(5-{3-cyano-4-[(1-methylethyl)oxy]phenyl}-1,2,4-oxadiazol-3-yl)-3-fluorophenyl]oxy}acetic acid (E23) (58 mg) as a white solid. δH (DMSO-d₆, 400 MHz): 1.32 (6H, d), 4.78 (2H, s), 4.90 (1H, m), 7.00 (2H, m), 7.47 (1H, d), 7.96 (1H, m), 8.30 (1H, dd), 8.40 (1H, d), 13.13 (1H, br s). δF (DMSO-d₆, 376 MHz): −105.9, −73.5. MS (ES): C₂₀H₁₆FN₃O₅ requires 397; found 398.2 (M+H⁺).

### EXAMPLE 24

### 4-{[4-(5-{3-cyano-4-[(1-methylethyl)oxy]phenyl}-1,2,4-oxadiazol-3-yl)phenyl]oxy}butanoic acid (E24)

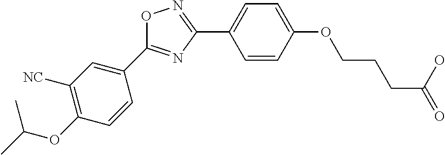

To a solution of ethyl 4-{[4-(5-{3-cyano-4-[(1-methylethyl)oxy]phenyl}-1,2,4-oxadiazol-3-yl)phenyl]oxy}butanoate (D53) (108 mg) in isopropanol (5 mL) and water (5.00 mL) was added 0.5 M NaOH (2.48 mL). The reaction solution was heated to 90° C. for 2 h. Isopropanol was removed in vacuo and the residue was acidified to pH=2-3, then extracted with ethyl acetate (3*10 mL). The combined organic phases were dried over sodium sulphate, concentrated and purified by Mass Directed Auto Prep to afford 4-{[4-(5-{3-cyano-4-[(1-methylethyl)oxy]phenyl}-1,2,4-oxadiazol-3-yl)phenyl]oxy}butanoic acid (E24) (62 mg) as a white solid. δH (DMSO-d₆, 400 MHz): 1.31 (611, d), 1.92 (2H, m), 2.34 (2H, t), 4.02 (2H, t), 4.90 (1H, m), 7.05 (2H, d), 7.46 (1H, d), 7.93 (2H, d), 8.30 (1H, dd), 8.39 (111, d), 12.09 (1H, br s). δF (DMSO-d₆, 376 MHz): −73.4. MS (ES): C₂₂H₂₁N₃O₅ requires 407; found 408.2 (M+H⁺).

### EXAMPLE 25

### {[4-(5-{3-cyano-4-[(1-methylethyl)oxy]phenyl}-1,2,4-oxadiazol-3-yl)phenyl]oxy}acetic acid (E25)

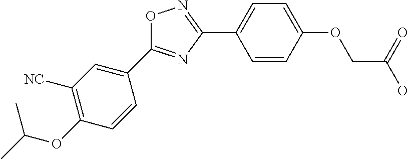

To a solution of ethyl {[4-(5-{3-cyano-4-[(1-methylethyl)oxy]phenyl}-1,2,4-oxadiazol-3-yl)phenyl]oxy}acetate (D54) (80 mg) in isopropanol (5 mL) and water (5.00 mL) was added 0.5 M NaOH (1.964 mL). The reaction solution was heated to 90° C. for 2 h. Isopropanol was removed in vacuo and the residue was acidified to pH=2-3, then extracted with ethyl acetate (3*10 mL). The combined organic phases were dried over sodium sulphate, concentrated and purified by Mass Directed Auto Prep to afford {[4-(5-{3-cyano-4-[(1-methylethyl)oxy]phenyl}-1,2,4-oxadiazol-3-yl)phenyl]oxy}acetic acid (E25) (37 mg) as a white solid. δH (DMSO-d₆, 400 MHz): 1.31 (6H, d), 4.73 (2H, s), 4.89 (1H, m), 7.05 (2H, d), 7.46 (1H, d), 7.94 (2H, d), 8.30 (1H, dd), 8.39 (1H, d), 13.03 (1H, br s). δF (DMSO-d₆, 376 MHz): −73.5. MS (ES): C₂₀H₁₇N₃O₅ requires 379; found 380.1 (M+H⁺).

### EXAMPLE 26

### (2-{[4-(5-{3-chloro-4-[(1-methylethyl)oxy]phenyl}-1,2,4-oxadiazol-3-yl)-3-ethylphenyl]oxy}ethyl)methylamine (E26)

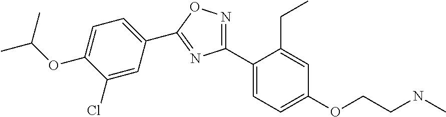

To a solution of 3-(4-[(2-bromoethyl)oxy]-2-ethylphenyl)-5-{3-chloro-4-[(1-methylethyl)oxy]phenyl}-1,2,4-oxadiazole (D63) (120 mg) in tetrahydrofuran (THF) (2 mL) was added methanamine (2 mL). The reaction solution was heated to 60° C. in a sealed tube for overnight. After filtration, the filtrate was concentrated and purified by Mass Directed Auto Prep to afford (2-{[4-(5-{3-chloro-4-[(1-methylethyl)oxy]phenyl}-1,2,4-oxadiazol-3-yl)-3-ethylphenyl]oxy}ethyl)methylamine (E26) (74 mg) as a white solid. δH (DMSO-d₆, 400 MHz): 1.14 (3H, t), 1.29 (6H, d), 2.61 (3H, s), 2.94 (2H, q), 3.33 (2H, s), 4.27 (2H, t), 4.81 (1H, m), 6.96 (2H, m), 7.37 (1H, d), 7.91 (1H, d), 8.02 (1H, dd), 8.09 (1H, d), 8.75 (2H, br s). δF (DMSO-d₆, 376 MHz): −73.8. MS (ES): C₂₂H₂₆ClN₃O₃ requires 415; found 416.2 (M+H⁺).

### EXAMPLE 27

### {[4-(5-{3-cyano-4-[(1-methylethyl)oxy]phenyl}-1,2,4-oxadiazol-3-yl)-3-ethylphenyl]oxy}acetic acid (E27)

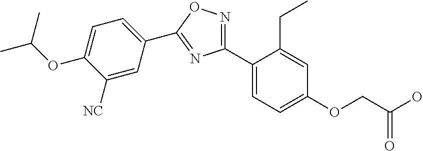

To a solution of ethyl {[4-(5-{3-cyano-4-[(1-methylethyl)oxy]phenyl}-1,2,4-oxadiazol-3-yl)-3-ethylphenyl]oxy}acetate (D66) (112 mg) in isopropanol (5 mL) and water (5.00 mL) was added 0.5 M NaOH (2.57 mL). The reaction solution was heated to 90° C. for 2 h. Isopropanol was removed in vacuo and the residue was added water (5 mL), acidified to pH=1-2 and extracted with ethyl acetate (3*10 mL). The combined organic phases were dried over sodium sulphate, concentrated and purified by Mass Directed Auto Prep to afford {[4-(5-{3-cyano-4-[(1-methylethyl)oxy]phenyl}-1,2,4-oxadiazol-3-yl)-3-ethylphenyl]oxy}acetic acid (E27) (60 mg) as a white solid. δH (DMSO-d₆, 400 MHz): 1.12 (3H, t), 1.32 (6H, d), 2.90 (2H, q), 4.72 (2H, s), 4.89 (1H, m), 6.89 (2H, m), 7.46 (1H, d), 7.85 (1H, d), 8.29 (1H, dd), 8.39 (1H, d), 13.05 (1H, br s). δF (DMSO-d₆, 376 MHz): −73.5. MS (ES): C₂₂H₂₁N₃O₅ requires 407; found 408.2 (M+H⁺).

### EXAMPLE 28

### {[4-(5-{3-chloro-4-[(1-methylethyl)oxy]phenyl}-1,2,4-oxadiazol-3-yl)-3-ethylphenyl]oxy}acetic acid (E28)

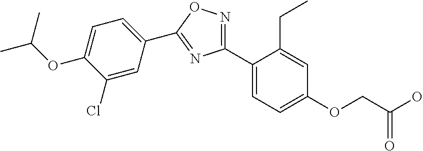

To a solution of ethyl {[4-(5-{3-chloro-4-[(1-methylethyl)oxy]phenyl}-1,2,4-oxadiazol-3-yl)-3-ethylphenyl]oxy}acetate (D67) (136 mg) in isopropanol (5 mL) and water (5.00 mL) was added NaOH (3.06 mL). The reaction solution was heated to 90° C. for 2 h. Isopropanol was removed in vacuo and the residue was added water (5 mL), acidified to pH=1-2 and extracted with ethyl acetate (3*10 mL). The combined organic phases were dried over sodium sulphate, concentrated and purified by Mass Directed Auto Prep to afford {[4-(5-{3-chloro-4-[(1-methylethyl)oxy]phenyl}-1,2,4-oxadiazol-3-yl)-3-ethylphenyl]oxy}acetic acid (E28) (61 mg) as a white solid. δH (DMSO-d₆, 400 MHz): 1.12 (3H, t), 1.29 (6H, d), 2.90 (2H, q), 4.72 (2H, s), 4.80 (1H, m), 6.88 (2H, m), 7.36 (1H, d), 7.85 (1H, d), 8.01 (1H, dd), 8.08 (1H, d), 13.07 (1H, br s). δF (DMSO-d₆, 376 MHz): −73.5. MS (ES): C₂₁H₂₁ClN₂O₅ requires 416; found 417.1 (M+H⁺).

### EXAMPLE 29

### 5-{[4-(5-{3-chloro-4-[(1-methylethyl)oxy]phenyl}-1,2,4-oxadiazol-3-yl)-3-ethylphenyl]oxy}pentanoic acid (E29)

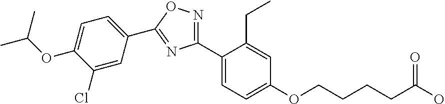

To a solution of ethyl 5-{[4-(5-{3-chloro-4-[(1-methylethyl)oxy]phenyl}-1,2,4-oxadiazol-3-yl)-3-ethylphenyl]oxy}pentanoate (D68) (163 mg) in isopropanol (5 mL) and water (5.00 mL) was added NaOH (3.35 mL). The reaction solution was heated to 90° C. for 2 h. Isopropanol was removed in vacuo and the residue was added water (5 mL), acidified to pH=1-2 and extracted with ethyl acetate (3*10 mL). The combined organic phases were dried over sodium sulphate, concentrated and purified by Mass Directed Auto Prep to afford 5-{[4-(5-{3-chloro-4-[(1-methylethyl)oxy]phenyl}-1,2,4-oxadiazol-3-yl)-3-ethylphenyl]oxy}pentanoic acid (E29) (59 mg) as a white solid. δH (DMSO-d₆, 400 MHz): 1.13 (3H, t), 1.29 (6H, d), 1.69 (4H, m), 2.25 (2H, t), 2.91 (2H, q), 3.99 (2H, t), 4.80 (1H, m), 6.87 (2H, m), 7.34 (1H, d), 7.85 (1H, d), 7.99 (1H, dd), 8.06 (1H, d), 12.02 (1H, br s). δF (DMSO-d₆, 376 MHz): −73.5. MS (ES): C₂₄H₂₇ClN₂O₅ requires 458; found 459.2 (M+H⁺).

### EXAMPLE 30

### 4-{[4-(5-{3-chloro-4-[(1-methylethyl)oxy]phenyl}-1,2,4-oxadiazol-3-yl)-3-ethylphenyl]oxy}butanoic acid (E30)

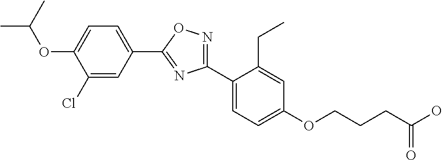

To a solution of ethyl 4-{[4-(5-{3-chloro-4-[(1-methylethyl)oxy]phenyl}-1,2,4-oxadiazol-3-yl)-3-ethylphenyl]oxy}butanoate (D69) (158 mg) in isopropanol (5 mL) and water (5.00 mL) was added 0.5 M NaOH (3.34 mL). The reaction solution was heated to 90° C. for 2 h. Isopropanol was removed in vacuo and the residue was added water (5 mL), acidified to pH=1-2 and extracted with ethyl acetate (3*10 mL). The combined organic phases were dried over sodium sulphate, concentrated and purified by Mass Directed Auto, Prep to afford 4-{[4-(5-{3-chloro-4-[(1-methylethyl)oxy]phenyl}-1,2,4-oxadiazol-3-yl)-3-ethylphenyl]oxy}butanoic acid (E30) (78 mg) as a white solid. δH (DMSO-d₆, 400 MHz): 1.13 (3H, t), 1.29 (6H, d), 1.90 (2H, m), 2.35 (2H, t), 2.91 (2H, q), 4.00 (2H, t), 4.80 (1H, m), 6.89 (2H, m), 7.35 (1H, d), 7.86 (1H, d), 8.00 (1H, dd), 8.07 (1H, d), 12.15 (1H, br s). δF (DMSO-d₆, 376 MHz): −73.5. MS (ES): C₂₃H₂₅ClN₂O₅ requires 444; found 445.2 (M+H⁺).

### EXAMPLE 31

### 5-{[4-(5-{3-cyano-4-[(1-methylethyl)oxy]phenyl}-1,2,4-oxadiazol-3-yl)-3-ethylphenyl]oxy}pentanoic acid (E31)

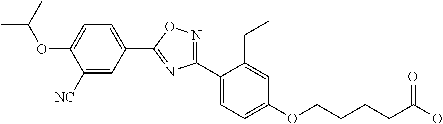

To a solution of ethyl 5-{[4-(5-{3-cyano-4-[(1-methylethyl)oxy]phenyl}-1,2,4-oxadiazol-3-yl)-3-ethylphenyl]oxy}pentanoate (D70) (137 mg) in isopropanol (2 mL) and water (2.000 mL) was added 0.5 M NaOH (2.87 mL). The reaction solution was heated to 90° C. for 2 h. Isopropanol was removed in vacuo and the residue was added water (5 mL), acidified to pH=1-2 and extracted with ethyl acetate (3*10 mL). The combined organic phases were dried over sodium sulphate, concentrated and purified by Mass Directed Auto Prep to afford 5-{[4-(5-{3-cyano-4-[(1-methylethyl)oxy]phenyl}-1,2,4-oxadiazol-3-yl)-3-ethylphenyl]oxy}pentanoic acid (E31) (44 mg) as a white solid. δH (DMSO-d₆, 400 MHz): 1.12 (3H, t), 1.32 (6H, d), 1.67 (4H, m), 2.25 (2H; t), 2.90 (2H, q), 3.99 (2H, t), 4.89 (1H, m), 6.88 (2H, m), 7.45 (1H, m), 7.85 (1H, m), 8.28 (1H, m), 8.37 (1H, m), 12.10 (1H, br s). δF (DMSO-d₆, 376 MHz): −73.5. MS (ES): C₂₆H₂₇N₃O₆ requires 449; found 450.3 (M+H⁺).

### EXAMPLE 32

### 4-{[4-(5-{3-cyano-4-[(1-methylethyl)oxy]phenyl}-1,2,4-oxadiazol-3-yl)-3-ethylphenyl]oxy}butanoic acid (E32)

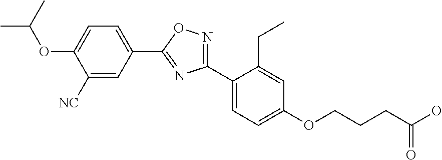

To a solution of ethyl 4-{[4-(5-{3-cyano-4-[(1-methylethyl)oxy]phenyl}-1,2,4-oxadiazol-3-yl)-3-ethylphenyl]oxy}butanoate (D71) (133 mg) in isopropanol (2 mL) and water (2.000 mL) was added 0.5 M NaOH (2.87 mL). The reaction solution was heated to 90° C. for 2 h. Isopropanol was removed in vacuo and the residue was added water (5 mL), acidified to pli=1-2 and extracted with ethyl acetate (3*10 mL). The combined organic phases were dried over sodium sulphate, concentrated and purified by Mass Directed Auto Prep to afford 4-{[4-(5-{3-cyano-4-[(1-methylethyl)oxy]phenyl}-1,2,4-oxadiazol-3-yl)-3-ethylphenyl]oxy}butanoic acid (E32) (70 mg) as a white solid. δH (DMSO-d₆, 400 MHz): 1.14 (3H, t), 1.32 (6H, d), 1.91 (2H, m), 2.35 (2H, t), 2.91 (2H, q), 4.00 (2H, t), 4.89 (1H, m), 6.89 (2H, m), 7.45 (1H, d), 7.85 (1H, d), 8.29 (1H, dd), 8.38 (1H, d), 12.13 (1H, br s). δF (DMSO-d₆, 376 MHz): −73.5. MS (ES): C₂₄H₂₆N₃O₆ requires 435; found 436.2 (M+H⁺).

### EXAMPLE 33

### 5-[3-(2-ethyl-4-{[2-(methylamino)ethyl]oxy}phenyl)-1,2,4-oxadiazol-5-yl]-2-[(1-methylethyl)oxy]benzonitrile (E33)

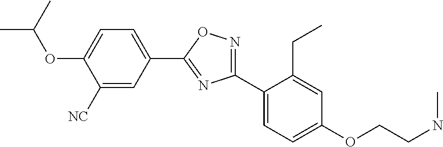

To a solution of 5-(3-{4-[(2-bromoethyl)oxy]-2-ethylphenyl}-1,2,4-oxadiazol-5-yl)-2-[(1-methylethyl)oxy]benzonitrile (D72) (110 mg) in tetrahydrofuran (THF) (2 mL) was added methanamine (2 mL). The reaction solution was stirred in a sealed tube at 35° C. for 2 d. After filtration, the filtrate was concentrated and purified by Mass Directed Auto Prep to afford 5-[3-(2-ethyl-4-{[2-(methylamino)ethyl]oxy}phenyl)-1,2,4-oxadiazol-5-yl]-2-[(1-methylethyl)oxy]benzonitrile (E33) (58 mg) as a white solid. δH (DMSO-d₆, 400 MHz): 1.14 (3H, t), 1.32 (6H, d), 2.62 (3H, s), 2.93 (2H, q), 3.34 (2H, m), 4.27 (2H, t), 4.91 (1H, m), 6.96 (2H, m), 7.48 (1H, d), 7.91 (1H, d), 8.31 (1H, dd), 8.40 (1H, d), 8.79 (2H, br s). δF (DMSO-d₆, 376 MHz): −73.6. MS (ES): C₂₃H₂₆N₄O₃ requires 406; found 407.2 (M+H⁺).

### EXAMPLE 34

### (4-{[4-(5-{3-chloro-4-[(1-methylethyl)oxy]phenyl}-1,2,4-oxadiazol-3-yl)-3-ethylphenyl]oxy}butyl)methylamine (E34)

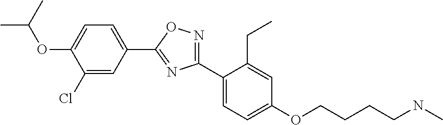

To a solution of 3-{4-[(4-bromobutyl)oxy]-2-ethylphenyl}-5-{3-chloro-4-[(1-methylethyl)oxy]phenyl}-1,2,4-oxadiazole (D73) (120 mg) in tetrahydrofuran (THF) (2 mL) was added methanamine (0.122 mL). The reaction solution was stirred in a sealed tube at 50° C. overnight. After filtration, the filtrate was concentrated and purified by Mass Directed Auto Prep to afford (4-{[4-(5-{3-chlord-4-[(1-methylethyl)oxy]phenyl}-1,2,4-oxadiazol-3-yl)-3-ethylphenyl]loxy}butyl)methylamine (E34) (81 mg) as a white solid. δH (DMSO-d₆, 400 MHz): 1.13 (3H, t), 1.29 (6H, d), 1.72 (4H, m), 2.52 (3H, t), 2.92 (4H, m), 4.02 (2H, t), 4.81 (1H, m), 6.90 (2H, m), 7.36 (1H, d), 7.88 (1H, dd), 8.01 (1H, dd), 8.08 (1H, d), 8.46 (2H, br s). δF (DMSO-d₆, 376 MHz): −73.7. MS (ES): C₂₄H₃₀ClN₃O₃ requires 443; found 444.2 (M+H⁺).

### EXAMPLE 35

### (3-{[4-(5-{3-chloro-4-[(1-methylethyl)oxy]phenyl}-1,2,4-oxadiazol-3-yl)-3-ethylphenyl]oxy}propyl)methylamine (E35)

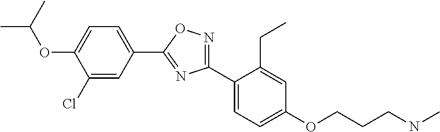

To a solution of 3-{4-[(3-bromopropyl)oxy]-2-ethylphenyl}-5-{3-chloro-4-[(1-methylethyl)oxy]phenyl}-1,2,4-oxadiazole (D74) (120 mg) in tetrahydrofuran (THF) (2 mL) was added methanamine (2 mL). The reaction solution was stirred in a sealed tube at 50° C. overnight. After filtration, the filtrate was concentrated and purified by Mass Directed Auto Prep to afford (3-{[4-(5-{3-chloro-4-[(1-methylethyl)oxy]phenyl}-1,2,4-oxadiazol-3-yl)-3-ethylphenyl]oxy}propyl)methylamine (E35) (64 mg) as a white solid. δH (DMSO-d₆, 400 MHz): 1.13 (3H, t), 1.29 (6H, d), 2.01 (2H, m), 2.55 (3H, s), 2.92 (2H, q), 3.03 (2H, t), 4.09 (2H, t), 4.82 (1H, m), 6.91 (2H, m), 7.37 (1H, d), 7.89 (1H, d), 8.03 (1H, dd), 8.09 (1H, d), 8.40 (2H, br s). δF (DMSO-d₆, 376 MHz): −73.5. MS (ES): C₂₃H₂₈ClN₃O₃ requires 429; found 430.2 (M+H⁺).

### EXAMPLE 36

### 5-[3-(2-chloro-4-[(2-(methylamino)ethygoxy)phenyl]-1,2,4-oxadiazol-5-yl]-2-[(1-methylethyl)oxy]benzonitrile (E36)

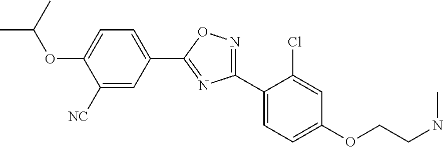

To the solution of 5-(3-{4-[(2-bromoethyl)oxy]-2-chlorophenyl}-1,2,4-oxadiazol-5-yl)-2-[(1-methylethyl)oxy]benzonitrile (D75) (100 mg) in tetrahydrofuran (THF) (2 mL) was added methanamine (2 mL). The reaction solution was stirred in a sealed tube for 2 d at 40° C. After filtration, the filtrate was concentrated and purified by Mass Directed Auto Prep to afford 5-[3-(2-chloro-4-{[2-(methylamino)ethyl]oxy}phenyl)-1,2,4-oxadiazol-5-yl]-2-[(1-methylethyl)oxy]benzonitrile (E36) (64 mg) as a white solid. δH (DMSO-d₆, 400 MHz): 1.31 (6H, d), 2.60 (3H, s), 3.32 (2H, m), 4.30 (2H, t), 4.91 (1H, m), 7.13 (1H, dd), 7.27 (1H, d), 7.49 (1H, d), 7.95 (1H, d), 8.32 (1H, dd), 8.43 (1H, d), 8.66 (2H, br s). δF (DMSO-d₆, 376 MHz): −73.5. MS (ES): C₂₁H₂₁ClN₄O₃ requires 412; found 413.1 (M+14⁺).

### EXAMPLE 37

### 5-[3-(2-chloro-4-{[3-(methylamino)propyl]oxy}phenyl)-1,2,4-oxadiazol-5-yl]-2-[(1-methylethyl)oxy]benzonitrile (E37)

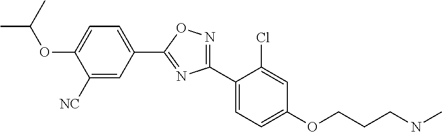

To the solution of 5-(3-{4-[(3-bromopropyl)oxy]-2-chlorophenyl}-1,2,4-oxadiazol-5-yl)-2-[(1-methylethyl)oxy]benzonitrile (D76) (100 mg) in tetrahydrofuran (THF) (2 mL) was added methanamine (2 mL). The reaction solution was stirred in a sealed tube for 2 d at room temperature. After filtration, the filtrate was concentrated and purified by Mass Directed Auto Prep to afford 5-[3-(2-chloro-4-{[3-(methylamino)propyl]oxy}phenyl)-1,2,4-oxadiazol-5-yl]-2-[(1-methylethyl)oxy]benzonitrile (E37) (72 mg) as a white solid. δH (DMSO-d₆, 400 MHz): 1.32 (6H, d), 2.00 (2H, m), 2.55 (3H, t), 3.02 (2H, m), 4.13 (2H, t), 4.91 (1H, m), 7.08 (1H, dd), 7.22 (1H, d), 7.49 (1H, d), 7.93 (1H, d), 8.32 (3H, m), 8.43 (1H, d). δF (DMSO-d₆, 376 MHz): −73.5. MS (ES): C₂₂H₂₃ClN₄O₃ requires 426; found 427.2 (M+H⁺).

### EXAMPLE 38

### 5-[3-(2-ethyl-4-{[4-(methylamino)butyl]oxy}phenyl)-1,2,4-oxadiazol-5-yl]-2-[(1-methylethyl)oxy]benzonitrile (E38)

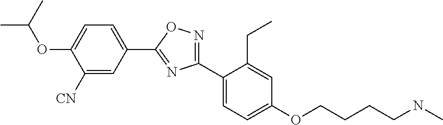

To the solution of 5-(3-{4-[(4-bromobutyl)oxy]-2-ethylphenyl}-1,2,4-oxadiazol-5-yl)-2-[(1-methylethyl)oxy]benzonitrile (D77) (110 mg) in tetrahydrofuran (THF) (2 mL) was added methanamine (2 mL). The reaction solution was stirred in a sealed tube for 2 d at room temperature. After filtration, the filtrate was concentrated and purified by Mass Directed Auto Prep to afford 5-[3-(2-ethyl-4-{[4-(methylamino)butyl]oxy}phenyl)-1,2,4-oxadiazol-5-yl]-2-[(1-methylethyl)oxy]benzonitrile (E38) (73 mg) as a white solid. δH (DMSO-d₆, 400 MHz): 1.13 (3H, t), 1.32 (6H, d), 1.72 (4H, m), 2.52 (3H, t), 2.93 (4H, m), 4.03 (2H, t), 4.91 (1H, m), 6.90 (2H, m), 7.48 (1H, d), 7.88 (1H, dd), 8.31 (1H, dd), 8.41 (3H, m). δF (DMSO-d₆, 376 MHz): −73.5. MS (ES): C₂₅H₃₀N₄O₃ requires 434; found 435.3 (M+H⁺.

### EXAMPLE 39

### 5-[3-(2-ethyl-4-{[3-(methylamino)propyl]oxy}phenyl)-1,2,4-oxadiazol-5-yl]-2-[(1-methylethyl)oxy]benzonitrile (E39)

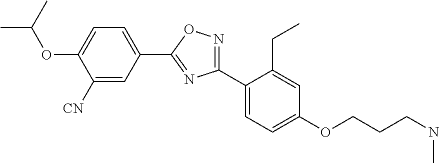

To the solution of 5-(3-{4-[(3-bromopropyl)oxy]-2-ethylphenyl}-1,2,4-oxadiazol-5-yl)-2-[(1-methylethyl)oxy]benzonitrile (D78) (110 mg) in tetrahydrofuran (THF) (2 mL) was added methanamine (2 mL). The reaction solution was stirred in a sealed tube for 2 d at room temperature. After filtration, the filtrate was concentrated and purified by Mass Directed Auto Prep to afford 5-[3-(2-ethyl-4-{[3-(methylamino)propyl]oxy}phenyl)-1,2,4-oxadiazol-5-yl]-2-[(1-methylethyl)oxy]benzonitrile (E39) (64 mg) as a white solid. δH (DMSO-d₆, 400 MHz): 1.13 (3H, t), 1.32 (6H, d), 2.02 (2H, m), 2.56 (3H, s), 2.93 (2H, m), 3.03 (2H, s), 4.09 (2H, t), 4.90 (1H, m), 6.91 (2H, m), 7.48 (1H, d), 7.89 (1H, d), 8.31 (1H, dd), 8.40 (1H, s), 8.53 (2H, br s). δF (DMSO-d₆, 376 MHz): −73.5. MS (ES): C₂₄H₂N₄O₃ requires 420; found 421.3 (M+H⁺).

### EXAMPLE 40

### (3-{[4-(5-{3-chloro-4-[(1-methylethyl)oxy]phenyl}-1,2,4-oxadiazol-3-yl)-3-methylphenyl]oxy}propyl)methylamine (E40)

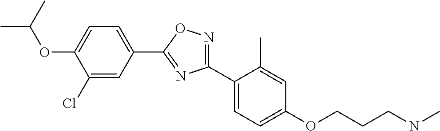

To the solution of 3-{4-[(3-bromopropyl)oxy]-2-methylphenyl}-5-{3-chloro-4-[(1-methylethyl)oxy]phenyl}-1,2,4-oxadiazole (D80) (160 mg) in tetrahydrofuran (THF) (2 mL) was added methanamine (1.718 mL). The resulting solution was stirred at 60° C. overnight. After filtration, the filtrate was concentrated and purified by Mass Directed Auto Prep to afford (3-{[4-(5-{3-chloro-4-[(1-methylethyl)oxy]phenyl}-1,2,4-oxadiazol-3-yl)-3-methylphenyl]oxy}propyl)methylamine (E40) (89 mg) as a white solid. δH (DMSO-d₆, 400 MHz): 1.29 (6H, d), 2.01 (2H, m), 2.55 (6H, m), 3.02 (2H, m), 4.08 (2H, t), 4.81 (1H, m), 6.91 (2H, m), 7.37 (1H, d), 7.94 (1H, d), 8.02 (1H, dd), 8.10 (1H, d), 8.42 (2H, br s). δF (DMSO-d₆, 376 MHz): −73.5. MS (ES): C₂₂H₂₆ClN₃O₃ requires 415; found 416.2 (M+H⁺).

### EXAMPLE 41

### 4-{[4-(3-{3-cyano-4-[(1-methylethyl)oxy]phenyl}-1,2,4-thiadiazol-5-yl)-3-ethylphenyl]oxy}butanoic acid (E41)

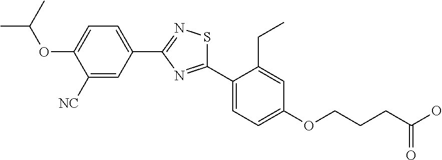

To a solution of ethyl 4-{[4-(3-{3-cyano-4-[(1-methylethyl)oxy]phenyl}-1,2,4-thiadiazol-5-yl)-3-ethylphenyl]oxy}butanoate (D82) (40 mg) in isopropanol (15 mL) and water (4 mL) stirred in air at room temperature was added 20% sodium hydroxide (0.5 mL) dropwise. The reaction mixture was stirred at room temperature for 4 h. The reaction mixture was acidified by conc. HCl to pH=7. The mixture was dried in vacuo and dissolved in DMF, after filtration, the filtrate was purified by Mass Directed Auto Prep to afford 4-{[4-(3-{3-cyano-4-[(1-methylethyl)oxy]phenyly}-1,2,4-thiadiazol-5-yl)-3-ethylphenyl]oxy}butanoic acid (E41) (9 mg). δH (DMSO-d₆, 400 MHz): 1.26 (3H, t), 1.37 (6H, d), 1.98 (2H, m), 2.41 (2H, t), 3.02 (2H, m), 4.10 (2H, t), 4.91 (1H, m), 6.99 (2H, m), 7.48 (1H, d), 7.97 (1H, dd), 8.48 (2H, m), 12.19 (1H, s). δF (DMSO-d₆, 376 MHz): −73.4. MS (ES): C₂₄H₂₆N₃O₄S requires 451; found 452.2 (M+H⁺).

### EXAMPLE 42

### 4-{[4-(5-{3-cyano-4-[(1-methylethyl)oxy]phenyl}-1,2,4-thiadiazol-3-yl)-3-ethylphenyl]oxy}butanoic acid (E42)

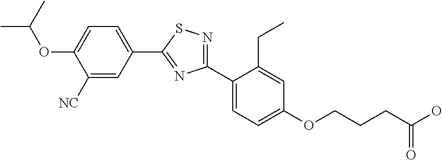

To a solution of ethyl 4-{[4-(5-{3-cyano-4-[(1-methylethyl)oxy]phenyl}-1,2,4-thiadiazol-3-yl)-3-ethylphenyl]oxy}butanoate (D84) (62 mg) in isopropanol (15 mL) and water (4 mL) stirred in air at room temperature was added 20% sodium hydroxide (0.2 mL) dropwise. The reaction mixture was stirred at room temperature overnight. The reaction mixture was acidified by AcOH to pH=7. The mixture was dried in vacuo and dissolved in DMF, after filtration, the filtrate was purified by Mass Directed Auto Prep to afford 4-{[4-(5-{3-cyano-4-[(1-methylethyl)oxy]phenyl}-1,2,4-thiadiazol-3-yl)-3-ethylphenyl]oxy}butanoic acid (E42) (24 mg). δH (DMSO-d₆, 400 MHz): 1.18 (3H, t), 1.37 (6H, d), 1.98 (2H, m), 2.41 (2H, t), 3.06 (2H, m), 4.07 (2H, t), 4.95 (1H, m), 6.93 (2H, m), 7.50 (1H, d), 8.04 (1H, d), 8.33 (1H, dd), 8.48 (1H, d), 12.18 (1H, s). MS (ES): C₂₄H₂₅N₃O₄S requires 451; found 452.2 (M+H⁺).

### EXAMPLE 43

### 4-{[4-(2-{3-cyano-4-[(1-methylethyl)oxy]phenyl}-1,3-thiazol-5-yl)-3-ethylphenyl]oxy}butanoic acid (E43)

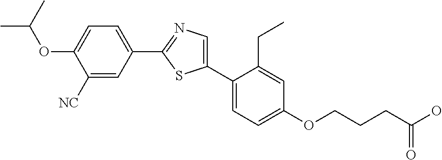

To a solution of ethyl 4-{[4-(2-{3-cyano-4-[(1-methylethyl)oxy]phenyl}-1,3-thiazol-5-yl)-3-ethylphenyl]oxy}butanoate (D87) (59 mg) in isopropanol (10 mL) and water (2 mL) stirred in air at room temperature was added 20% sodium hydroxide (0.5 mL) dropwise. The reaction mixture was stirred at room temperature overnight. The reaction mixture was acidified by AcOH to pH=7. The mixture was dried in vacuo and dissolved in DMF, after filtration, the filtrate was purified by Mass Directed Auto Prep to afford 4-{[4-(2-{3-cyano-4-[(1-methylethyl)oxy]phenyl}-1,3-thiazol-5-yl)-3-ethylphenyl]oxy}butanoic acid (E43) (19 mg). δH (DMSO-d₆, 400 MHz): 1.13 (3H, t), 1.36 (6H, d), 1.95 (2H, m), 2.38 (2H, t), 2.69 (2H, m), 4.03 (2H, t), 4.89 (1H, m), 6.87 (1H, dd), 6.95 (1H, d), 7.33 (1H, d), 7.42 (1H, d), 7.82 (1H, s), 8.21 (2H, m), 11.89 (1H, br s). MS (ES): C₂₅H₂₅N₂O₄S requires 450; found 451.2 (M+H⁺).

### EXAMPLE 44

### 5-[5-(2-ethyl-4-{[3-(methylamino)propyl]oxy}phenyl)-1,3-thiazol-2-yl]-2-[(1-methylethyl)oxy]benzonitrile (E44)

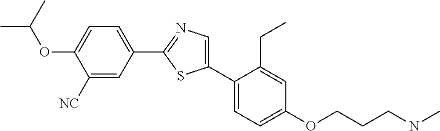

To a solution of 1,1-dimethylethyl (3-{[4-(2-{3-cyano-4-[(1-methylethyl)oxy]phenyl}-1,3-thiazol-5-yl)-3-ethylphenyl]oxy}propyl)methylcarbamate (D88) (40 mg) in dichloromethane (DCM) (5 mL) stirred in air at room temperature was added TFA (0.1 mL) dropwise. The reaction mixture was stirred at RT overnight. The mixture was dried in vacuo and purified by Mass Directed Auto Prep to afford 5-[5-(2-ethyl-4-{[3-(methylamino)propyl]oxy}phenyl)-1,3-thiazol-2-yl]-2-[(1-methylethyl)oxy]benzonitrile (E44) (25 mg) as a white solid. δH (DMSO-d₆, 400 MHz): 1.14 (3H, t), 1.36 (6H, d), 2.05 (2H, m), 2.61 (3H, s), 2.71 (2H, m), 3.07 (2H, t), 4.11 (2H, t), 4.90 (1H, m), 6.89 (1H, dd), 6.95 (1H, d), 7.36 (1H, d), 7.43 (1H, d), 7.83 (1H, s), 8.19 (1H, dd), 8.25 (1H, d), 8.35 (2H, br s). δF (DMSO-d₆, 376 MHz): −73.4. MS (ES): C₂₆H₂₉N₃O₂S requires 435; found 436.2 (M+H⁺).

### S1P1 Tango Assay

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were suspended in assay medium (Invitrogen Freestyle Expression Medium) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (S1P) (row 1, column 1-10) in a Corning black-well, clear bottom 96-well plate. Cells were incubated at 37° C., 5% CO₂ for 44-48 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free control wells.

After incubation at 37° C., 5% CO₂ for 5 h, 25 μl of 6× substrate mixture (6 μl Solution A (1 mg LiveBLAzer™-FRET B/G substrate (CCF4-AM) in 912 μl DMSO) plus 60 μl Solution B plus 934 μl Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted Green emission values. The dose response curve is based on sigmoidal dose-response model. All ratio data was normalized based upon the maximum emission ratio of positive control (S1P) and minimum emission ratio of negative control (DMSO) on each plate. The intrinsic activity (IA) of each compound would be the normalized percentage of its maximum response after curve fitting.

### EXAMPLES 1 TO 12 HAD A PEC50>5 IN THIS ASSAY

### S1P1 Tango Assay—384 Well Format

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were harvested from growth medium and passaged into assay medium (Invitrogen Freestyle Expression Medium). The cells were starved for 24 hours at 37° C., 5% CO₂, harvested and resuspended in assay medium at a density of ˜200,000 cells/ml.

All test compounds were dissolved in DMSO at a concentration of 10 mM and were prepared in 100% DMSO to provide 10 point dose response curves. Test compounds prepared by Bravo (Velocity 11) were added to wells in columns 2-11 and 13-22; DMSO was added to wells in columns 12 and 23 as unstimulated controls and assay medium was added to wells in columns 1 and 24 as cell-free controls. An S1P1 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C./5% CO₂.

10 μl of 6× substrate mixture (LiveBLAzer™-FRET B/G substrate (CCF4-AM) Cat # K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Examples 13 and 44 had a pEC50≧6 in this assay. Examples 14, 23, 25 and 34 had a pEC50≧7 in this assay. Examples 5, 6, 9, 17 to 20, 26, 33, 35, 38, 40 and 42 had a pEC50≧8 in this assay. Examples 2, 3, 8, 11, 12, 15, 16, 21, 24, 27 to 29, 36, 37, 39, 41 and 43 had a pEC50≧9 in this assay. Examples 1, 7, 10, 22, 30 and 30 to 32 had a pEC50≧10 in this assay.

### S1P3 GeneBlazer Assay

GeneBLAzer EDG3-Ga15-NFAT-bla HEK 293T cells (contain the human Endothelial Differentiation G-protein Coupled Receptor 3 (EDG3) and a beta-lactamase reporter gene under control of a NFAT response element and a promiscuous G Protein, Ga15, stably integrated into the GeneBLAzer Ga15-NFAT-bla HEK 293T cell line) were suspended in assay medium (99% DMEM, 1% Dialyzed FBS, 0.1 mM NEAA, 25 mM HEPES (pH 7.3), 100 U/rill penicillin, 100 μg/mlstreptomycin) at a density of 312, 500 cells/ml. Add 100 μl/well of the assay medium to the cell-free control wells (column 12) and 100 W/well of the cell suspension to the test compound wells (row 2-8, column 1-10), the unstimulated control wells (DMSO) (column 11), and stimulated control wells (S1P) (row 1, column 1-10) in a Corning black-well, clear bottom 96-well plate. Cells were incubated at 37° C., 5% CO2 for 24 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free Control wells.

After incubation at 37° C., 5% CO2 for 5 h, 25 μl of 6× substrate mixture (6 μl Solution A (1 mg LiveBLAzer™-FRET B/G Substrate (CCF4-AM) in 91411 DMSO) plus 60 μl Solution B plus 934 μl, Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted green emission values. The dose response curve is based on sigmoidal dose-response model. All ratio data was normalized based upon the maximum emission ratio of positive control (S1P) and minimum emission ratio of negative control (DMSO) on each plate. The intrinsic activity (IA) of each compound would be the normalized percentage of its maximum response after curve fitting.

Exemplified compounds of the invention had a pEC50<5.

1. A compound of formula (I) or a salt thereof: 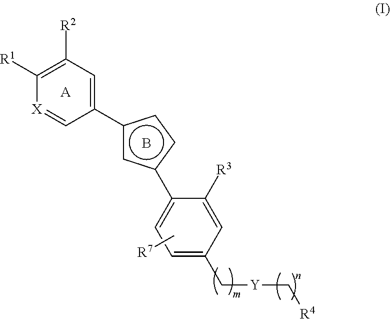
wherein
X is CH or N;
B is a 5-membered heteroaryl ring selected from: 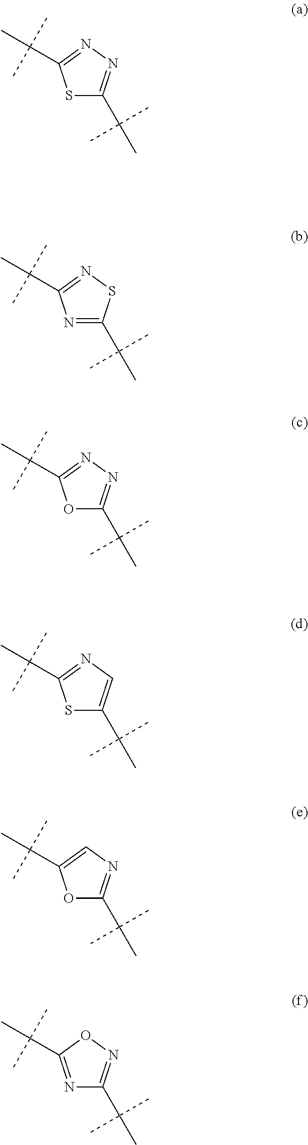
Y is O or S;
m is 0 to 4;
n is 1 to 4;
R¹ is C₍₁₋₄₎alkoxy;
R² is cyano or chloro;
R³ is C₍₁₋₅₎alkyl, C₍₁₋₅₎alkoxy, halogen, hydrogen, trifluoromethyl, or CN;
R⁴ is COOH, NR⁵R⁶ or OR⁸;
one of R⁵ and R⁶ is C₍₁₋₃₎alkyl and the other is hydrogen or C₍₁₋₃₎alkyl;
R⁸ is C₍₁₋₃₎alkyl;
R⁷ is C₍₁₋₃₎alkyl, C₍₁₋₃₎alkoxy, halogen or hydrogen; and
when m is 1 to 4 and n is 1 to 4 the alkyl groups which they represent may be optionally substituted by C₍₁₋₃₎alkyl or OH.

2. A compound of formula (I) or a salt thereof, wherein:
X is CH;
B is (f);
Y is O;
m is 0;
n is 1-4;
R¹ is C₍₁₋₄₎alkoxy;
R² is cyano or chloro;
R³ is C₍₁₋₃₎alkyl, C₍₁₋₃₎alkoxy, halogen or hydrogen;
R⁴ is COOH, NR⁵R⁶ or OR⁸;
R⁵ is hydrogen or methyl;
R⁶ is methyl;
R⁷ is hydrogen; and
R⁸ is methyl.

3. A compound selected from:
4-{[4-(5-{3-Chloro-4-[(1-methylethyl)oxy]phenyl}-1,2,4-oxadiazol-3-yl)-3-methylphenyl]oxy}butanoic acid
5-{[4-(5-{3-Chloro-4-[(1-methylethyl)oxy]phenyl}-1,2,4-oxadiazol-3-yl)-3-methylphenyl]oxy}pentanoic acid
{[4-(5-{3-Chloro-4-[(1-methylethyl)oxy]phenyl}-1,2,4-oxadiazol-3-yl)-3-methylphenyl]oxy}acetic acid
3-{[4-(5-{3-Chloro-4-[(1-methylethyl)oxy]phenyl}-1,2,4-oxadiazol-3-yl)-3-methylphenyl]oxy}-N,N-dimethyl-1-propanamine
2-{[4-(5-{3-Chloro-4-[(1-methylethyl)oxy]phenyl}-1,2,4-oxadiazol-3-yl)-3-methylphenyl]oxy}-N-methylethanamine
5-{3-Chloro-4-[(1-methylethyl)oxy]phenyl}-3-(2-methyl-4-{[2-(methyloxy)ethyl]oxy}phenyl)-1,2,4-oxadiazole
5-{[3-Chloro-4-(5-{3-cyano-4-[(1-methylethyl)oxy]phenyl}-1,2,4-oxadiazol-3-yl)phenyl]oxy}pentanoic acid
5-{[4-(5-{3-Cyano-4-[(1-methylethyl)oxy]phenyl}-1,2,4-oxadiazol-3-yl)phenyl]oxy}pentanoic acid
{[3-Chloro-4-(5-{3-cyano-4-[(1-methylethyl)oxy]phenyl}-1,2,4-oxadiazol-3-yl)phenyl]oxy}acetic acid
4-{[3-Chloro-4-(5-{3-cyano-4-[(1-methylethyl)oxy]phenyl}-1,2,4-oxadiazol-3-yl)phenyl]oxy}butanoic acid
5-{[4-(5-{3-Cyano-4-[(1-methylethyl)oxy]phenyl}-1,2,4-oxadiazol-3-yl)-3-fluorophenyl]oxy}pentanoic acid
5-{[4-(5-{3-Cyano-4-[(1-methylethyl)oxy]phenyl}-1,2,4-oxadiazol-3-yl)-3-(methyloxy)phenyl]oxy}pentanoic acid
5-[5-(2-ethyl-4-{[3-(methylamino)propyl]oxy}phenyl)-1,3,4-thiadiazol-2-yl]-2-[(1-methylethyl)oxy]benzonitrile
5-[5-(2-ethyl-4-{[4-(methylamino)propyl]oxy}phenyl)-1,3,4-oxadiazol-2-yl]-2-[(1-methylethyl)oxy]benzonitrile
4-{[4-(5-{3-cyano-4-[(1-methylethyl)oxy]phenyl}-1,3,4-oxadiazol-2-yl)-3-ethylphenyl]oxy}butanoic acid
4-{[4-(5-{3-cyano-4-[(1-methylethyl)oxy]phenyl}-1,3,4-thiadiazol-2-yl)-3-ethylphenyl]oxy}butanoic acid
5-[3-(2-chloro-4-{[4-(dimethylamino)butyl]oxy}phenyl)-1,2,4-oxadiazol-5-yl]-2-[(1-methylethyl)oxy]benzonitrile
5-[3-[2-chloro-4-({4-[(1-methylethyl)amino]butyl}oxy)phenyl]-1,2,4-oxadiazol-5-yl]-2-[(1-methylethyl)oxy]benzonitrile
5-[3-(2-chloro-4-{[4-(propylamino)butyl]oxy}phenyl)-1,2,4-oxadiazol-5-yl]-2-[(1-methylethyl)oxy]benzonitrile
5-[3-(2-chloro-4-{[4-(ethylamino)butyl]oxy}phenyl)-1,2,4-oxadiazol-5-yl]-2-[(1-methylethyl)oxy]benzonitrile
5-[3-(2-chloro-4-{[4-(methylamino)butyl]oxy}phenyl)-1,2,4-oxadiazol-5-yl]-2-[(1-methylethyl)oxy]benzonitrile
4-{[4-(5-{3-cyano-4-[(1-methylethyl)oxy]phenyl}-1,2,4-oxadiazol-3-yl)-3-fluorophenyl]oxy}butanoic acid
{[4-(5-{3-cyano-4-[(1-methylethyl)oxy]phenyl}-1,2,4-oxadiazol-3-yl)-3-fluorophenyl]oxy}acetic acid
4-{[4-(5-{3-cyano-4-[(1-methylethyl)oxy]phenyl}-1,2,4-oxadiazol-3-yl)phenyl]oxy}butanoic acid
{[4-(5-{3-cyano-4-[(1-methylethyl)oxy]phenyl}-1,2,4-oxadiazol-3-yl)phenyl]oxy}acetic acid
(2-{[4-(5-{3-chloro-4-[(1-methylethyl)oxy]phenyl}-1,2,4-oxadiazol-3-yl)-3-ethylphenyl]oxy}ethyl)methylamine
{[4-(5-{3-cyano-4-[(1-methylethyl)oxy]phenyl}-1,2,4-oxadiazol-3-yl)-3-ethylphenyl]oxy}acetic acid
{[4-(5-{3-chloro-4-[(1-methylethyl)oxy]phenyl}-1,2,4-oxadiazol-3-yl)-3-ethylphenyl]oxy}acetic acid
5-{[4-(5-{3-chloro-4-[(1-methylethyl)oxy]phenyl}-1,2,4-oxadiazol-3-yl)-3-ethylphenyl]oxy}pentanoic acid
4-{[4-(5-{3-chloro-4-[(1-methylethyl)oxy]phenyl}-1,2,4-oxadiazol-3-yl)-3-ethylphenyl]oxy}butanoic acid
5-{[4-(5-{3-cyano-4-[(1-methylethyl)oxy]phenyl}-1,2,4-oxadiazol-3-yl)-3-ethylphenyl]oxy}pentanoic acid
4-{[4-(5-{3-cyano-4-[(1-methylethyl)oxy]phenyl}-1,2,4-oxadiazol-3-yl)-3-ethylphenyl]oxy}butanoic acid
5-[3-(2-ethyl-4-{[2-(methylamino)ethyl]oxy}phenyl)-1,2,4-oxadiazol-5-yl]-2-[(1-methylethyl)oxy]benzonitrile
(4-{[4-(5-{3-chloro-4-[(1-methylethyl)oxy]phenyl}-1,2,4-oxadiazol-3-yl)-3-ethylphenyl]oxy}butyl)methylamine
(3-{[4-(5-{3-chloro-4-[(1-methylethyl)oxy]phenyl}-1,2,4-oxadiazol-3-yl)-3-ethylphenyl]oxy}propyl)methylamine
5-[3-(2-chloro-4-{[2-(methylamino)ethyl]oxy}phenyl)-1,2,4-oxadiazol-5-yl]-2-[(1-methylethyl)oxy]benzonitrile
5-[3-(2-chloro-4-{[3-(methylamino)propyl]oxy}phenyl)-1,2,4-oxadiazol-5-yl]-2-[(1-methylethyl)oxy]benzonitrile
5-[3-(2-ethyl-4-{[4-(methylamino)butyl]oxy}phenyl)-1,2,4-oxadiazol-5-yl]-2-[(1-methylethyl)oxy]benzonitrile
5-[3-(2-ethyl-4-{[3-(methylamino)propyl]oxy}phenyl)-1,2,4-oxadiazol-5-yl]-2-[(1-methylethyl)oxy]benzonitrile
(3-{[4-(5-{3-chloro-4-[(1-methylethyl)oxy]phenyl}-1,2,4-oxadiazol-3-yl)-3-methylphenyl]oxy}propyl)methylamine
4-{[4-(3-{3-cyano-4-[(1-methylethyl)oxy]phenyl}-1,2,4-thiadiazol-5-yl)-3-ethylphenyl]oxy}butanoic acid
4-{[4-(5-{3-cyano-4-[(1-methylethyl)oxy]phenyl}-1,2,4-thiadiazol-3-yl)-3-ethylphenyl]oxy}butanoic acid
4-{[4-(2-{3-cyano-4-[(1-methylethyl)oxy]phenyl}-1,3-thiazol-5-yl)-3-ethylphenyl]oxy}butanoic acid
5-[5-(2-ethyl-4-{[3-(methylamino)propyl]oxy}phenyl)-1,3-thiazol-2-yl]-2-[(1-methylethyl)oxy]benzonitrile and salts thereof.

4. Use of a compound according to any one of claims 1 to 3 for the treatment of conditions or disorders mediated by S1P1 receptors.

5. Use according to claim 4, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

6. Use according to claim 5, wherein the condition is multiple sclerosis.

7. Use of a compound according to any one of claims 1 to 3 to manufacture a medicament for use in the treatment of conditions or disorders mediated by S1P1 receptors.

8. Use according to claim 7, wherein the condition or disorder multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

9. Use according to claim 8, wherein the condition is multiple sclerosis.

10. A pharmaceutical composition comprising a compound of formula (I) or a pharmaceutically acceptable salt thereof according to any one of claims 1 to 3.

11. A method of treatment for conditions or disorders in mammals including humans which can be mediated via the S1P1 receptor.

12. A method of treatment according to claim 11, wherein the condition is multiple sclerosis.
